(19) United States
(12) Patent Application Publication
(10) Pub. No.: US 2014/0127215 A1
(43) Pub. Date: May 8, 2014

(54) CLOSTRIDIUM DIFFICILE ANTIGENS

(71) Applicants: Jody Berry, Winnipeg (CA); Darrell Johnstone, Winnipeg (CA); Bonnie Tighe, Winnipeg (CA); Marianela Lopez, Winnipeg (CA); Joyee George, Winnipeg (CA); Xiaobing Han, Winnipeg (CA)

(72) Inventors: Jody Berry, Winnipeg (CA); Darrell Johnstone, Winnipeg (CA); Bonnie Tighe, Winnipeg (CA); Marianela Lopez, Winnipeg (CA); Joyee George, Winnipeg (CA); Xiaobing Han, Winnipeg (CA)

(73) Assignee: CANGENE CORPORATION, Winnipeg (CA)

(21) Appl. No.: 13/976,530

(22) PCT Filed: Dec. 29, 2011
(86) PCT No.: PCT/US11/67806

§ 371 Date: Jan. 21, 2014

Related U.S. Application Data
(60) Provisional application No. 61/427,997, filed on Dec. 29, 2010.

(51) Int. Cl. C07K 16/12; A61K 45/06; C07K 16/40; A61K 39/40

(52) U.S. Cl. CPC C07K 16/1282; A61K 39/40; A61K 45/06; C07K 16/40
USPC 424/139.1; 530/387.9; 530/387.3

(57) ABSTRACT

Compositions and methods for the treatment or prevention of Clostridium difficile infection in a vertebrate subject are provided. The methods provide administering a composition to the vertebrate subject in an amount effective to reduce or eliminate or prevent relapse of Clostridium difficile bacterial infection and/or induce an immune response to the protein. Methods for the treatment or prevention of Clostridium difficile infection in a vertebrate are also provided.

### FIELD

The invention relates to compositions and methods for the treatment or prevention of infection by the Gram-positive bacteria, Clostridium difficile, in a vertebrate subject. Methods are provided for administering a protein to the vertebrate subject in an amount effective to reduce, eliminate, or prevent relapse from infection. Methods for the treatment or prevention of Clostridium difficile infection in an organism are provided.

### BACKGROUND

Clostridium difficile is a commensal Gram-positive bacterium of the human intestine present in 2-5% of the population. C. difficile has a dimorphic life cycle, capable of existing as a dormant, but yet infectious spore, and as a metabolically active toxin-producing vegetative cell. The presence of low numbers of C. difficile in the intestine is asymptomatic; however, bacterial overgrowth can result in severe and life threatening disease, especially in the elderly. Overgrowth by C. difficile can occur when the normal gut flora is is eradicated by antibiotic treatment. Thus, C. difficile is a major cause of antibiotic-associated diarrhea and can lead to pseudomembranous colitis, a generalized inflammation of the colon. Pathogenic C. difficile strains produce several known toxins. Two such toxins, entrotoxin (toxin A) and cytotoxin (toxin B) are responsible for the diarrhea and inflammation seen in infected patients.

Hospitalization or residence in a nursing home increases the risk for C. difficile infection. The rate of C. difficile acquisition has been estimated to be 13% in patients with hospital stays of up to 2 weeks, and 50% in those with hospital stays of longer than 4 weeks. Thus, C. difficile is a common nosocomial pathogen and a major cause of morbidity and mortality among hospitalized patients through the world. Because this organism forms heat-resistant spores, C. difficile can remain in the hospital or nursing home environment for long periods of time. Once spores are ingested, they survive passage through the stomach due to their acid resistance. Once in the colon, spores can germinate into vegetative cells upon exposure to bile acids.

Recurrence of C. difficile infection after an initial treatment is a common problem, as relapse of the disease occurs in 25% of patients treated for a first episode of infection. This is largely due to the fact that the organism is able to remain in a dormant, antiobiotic-resistant state as a spore.

Current therapies for treatment of C. difficile infection target the vegetative phase of the organism's life cycle. Among these treatments are antibiotics such as vanomycin or metronidazole. The use of fluoroquinolone antibiotics, such as ciprofloxacin and levofloxacin, has unfortunately led to the emergence of new, highly virulent, and antibiotic resistant strains of C. difficile. Other treatments, particularly for prevention of relapse, include prophylactic approaches such as the use of probiotics to restore the gut flora with non-pathogenic organisms such as Lactobacillus acidophilus or Saccharomyces boulardii. Typhimurium-based live vaccines have been developed through the identification of mutations affecting metabolic functions or essential virulence factors. Clin. Microbiol. Rev. 5 (1992) 328-342.

Attempts at vaccines to date have focused on the A and B toxins and vegetative cell surface proteins (SLPAs), all proteins produced by metabolically active bacteria. Thus, all current therapies address primary infection by vegetative stage bacteria, but do not target relapse from the dormant, but still infectious, spores. In light of the potential emergence of infectious diseases caused by increasingly toxic and drug-resistant strains of C. difficile, there remains an unmet need for an effective vaccine composition or antibody treatment for treating or preventing the occurrence of C. difficile associated disease, and its relapse, based on targeting of the recalcitrant spore phase of the life cycle of this organism.

### SUMMARY

Described herein are compositions and methods for the treatment or prevention of Clostridium difficile infection in a vertebrate subject.

In a first aspect, the present invention provides compositions containing an antibody or fragment that binds to a C. difficile spore polypeptide or fragment, where the spore polypeptide or fragment can be BclA1, BclA2, BclA3, Alr, SlpA paralogue, SlpA HMW, CD1021, IunH, Fe-Mn-SOD, or FliD.

In a second aspect, the present invention provides compositions containing an antibody or fragment that binds to a C. difficile spore polypeptide or fragment, where the spore polypeptide or fragment can have an amino acid sequence at least 80-95% identical to the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In a third aspect, the present invention provides an isolated antibody or fragment that binds to a C. difficile spore polypeptide or fragment, where the polypeptide or fragment can be BclA1, BclA2, BclA3, Alr, SlpA paralogue, SlpA HMW, CD1021, IunH, Fe-Mn-SOD, or FliD.

In a fourth aspect, the present invention provides an antibody or fragment that binds to a C. difficile spore polypeptide or fragment, where the polypeptide or fragment can have an amino acid sequence at least 80-95% identical to the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In various embodiments of the first four aspects, the the antibody or fragment can be a polyclonal antibody, a monoclonal antibody, a human antibody, a whole immunoglobulin molecule, an scFv; a chimeric antibody; a Fab fragment; an F(ab′)2; or a disulfide linked Fv.

In other embodiments of the the first four aspects, the antibody or fragment can have a heavy chain immunoglobulin constant domain, which can be a human IgM constant domain; a human IgG1 constant domain, a human IgG2 constant domain, a human IgG3 constant domain, a human IgG4 constant domain, or a human IgA1/2 constant domain.

In other embodiments of the first four aspects, the antibody or fragment can have a light chain immunoglobulin constant domain, which can be a human Ig kappa constant domain or a human Ig lambda constant domain.

In yet further embodiments of the first four aspects, the antibody or fragment can bind to an antigen with an affinity constant (Kaff) of at least 1×10⁹ M or at least 1×10¹⁰ M.

In additional embodiments of the first four aspects, the antibody or fragment thereof can inhibit or delay spore germination.

In some embodiments of the first and second aspects, the composition can also contain an antibody that binds to C. difficile toxin A, toxin B, or a combination of antibodies that bind toxin A and toxin B. In additional embodiments of the first and second aspects, the composition can also contain an antibiotic, such as metronidazole or vanomycin.

The compositions of the first four aspects can be used in a method of treatment of C. difficile associated disease by administration to a subject in need of such treatment an amount of the composition effective to reduce or prevent the disease, which can be an amount in the range of 1 to 100 milligrams per kilogram of the subject's body weight The compositions can be administered intravenously (IV), subcutaneously (SC), intramuscularly (IM), or orally.

In other aspects of the first four embodiments, the compositions can be used in a method of passive immunization by administration to an animal of an effective amount of the compositions.

In a fifth aspect, the present invention provides a method of inducing an immune response in a subject by administering to the subject an amount of a C. difficile spore polypeptide or fragment or variant, which can be BclA1, BclA2, BclA3, Alr, SlpA paralogue, SlpA HMW, CD1021, IunH, Fe-Mn-SOD, or FliD, and a pharmaceutically acceptable adjuvant in an amount effective to induce an immune response in the subject.

In a sixth aspect, the present invention provides a method of inducing an immune response in a subject by administering to the subject a C. difficile spore polypeptide or fragment or variant, which can have an amino acid sequence at least 80-95% identical to the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and a pharmaceutically acceptable adjuvant in an amount effective to induce an immune response in the subject.

In a seventh aspect, the present invention provides a method of reducing or preventing C. difficile infection in a subject in need of treatment by administering to the subject an amount of a C. difficile spore polypeptide or fragment or variant, which can be BclA1, BclA2, BclA3, Alr, SlpA paralogue, SlpA HMW, CD1021, IunH, Fe-Mn-SOD, or FliD, and a pharmaceutically acceptable adjuvant in an amount effective to reduce or prevent infection in the subject.

In an eighth aspect, the present invention provides a method of reducing or preventing C. difficile infection in a subject in need of such treatment by administering to the subject a C. difficile spore polypeptide or fragment, which can have an amino acid sequence at least 80-95% identical to the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and a pharmaceutically acceptable adjuvant in an amount effective to reduce or prevent infection in the subject.

In various embodiments of the fifth through eighth aspects, the pharmaceutically acceptable adjuvant is interleukin 12 or a heat shock protein. In other embodiments, the administration is oral, intranasal, intravenous, or intramuscular. In other embodiments, the variant is a mutant, which can be a fusion protein. The fusion protein can contain the sequence of C. difficile toxins A or B, for example, the N-terminal catalytic domain of TcdA, the N-terminal catalytic domain of TcdB, C-terminal fragment 4 of TcdB, or the C-terminal receptor binding fragment of TcdA. Alternatively, the fusion protein can be a fusion of any one of the proteins BclA1, BclA2, BclA3, Alr, SlpA paralogue, SlpA HMW, CD1021, IunH, Fe-Mn-SOD, or FliD, and fragments thereof, or a protein having the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and fragments thereof, with another member of the group of proteins.

In a ninth aspect, the present invention provides a composition containing an effective immunizing amount of an isolated polypeptide or fragment or variant and a pharmaceutically acceptable carrier, where the composition is effective in a subject to induce an immune response to a C. difficile infection, and where the isolated polypeptide or fragment or variant contains a C. difficile spore polypeptide or fragment, which can be BclA1, BclA2, BclA3, Alr, SlpA paralogue, SlpA HMW, CD1021, IunH, Fe-Mn-SOD, or FliD.

In a tenth aspect, the present invention provides a composition containing an effective immunizing amount of an isolated polypeptide or fragment or variant and a pharmaceutically acceptable carrier, where the composition is effective in a subject to induce an immune response to a C. difficile infection, and where the isolated polypeptide or fragment or variant has an amino acid sequence at least 80-95% identical to the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In various embodiments of the ninth and tenth aspects, the composition further contains a pharmaceutically acceptable adjuvant, which can be an oil-in-water emulsion, ISA-206, Quil A, interleukin 12 or a heat shock protein. In further embodiments of these aspects, the variant is a mutant, which can be a fusion protein. The fusion protein can contain the sequence of C. difficile toxins A or B, for example, the N-terminal catalytic domain of TcdA, the N-terminal catalytic domain of TcdB, C-terminal fragment 4 of TcdB, or the C-terminal receptor binding fragment of TcdA. Alternatively, the fusion protein can be a fusion of any one of the proteins BclA1, BclA2, BclA3, Alr, SlpA paralogue, SlpA HMW, CD1021, IunH, Fe-Mn-SOD, or FliD, and fragments thereof, or a protein having the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and fragments thereof, with another member of the group of proteins.

In an eleventh aspect, the present invention provides a method of reducing or preventing C. difficile infection in a subject in need of such treatment by administering to the subject an amount of a nucleic acid encoding a C. difficile spore polypeptide or fragment or variant, which can be BclA1, BclA2, BclA3, Alr, SlpA paralogue, SlpA HMW, CD1021, IunH, Fe-Mn-SOD, or FliD, and a pharmaceutically acceptable adjuvant in an amount effective to reduce or prevent infection in the subject.

In an twelfth aspect, the present invention provides a method of reducing or preventing C. difficile infection in a subject in need of such treatment by administering to the subject an amount of a nucleic acid encoding a C. difficile spore polypeptide or fragment or variant, having an amino acid sequence at least 80-95% identical to the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and a pharmaceutically acceptable adjuvant in an amount effective to reduce or prevent infection in the subject.

In various embodiments of the eleventh and twelfth aspects, the pharmaceutically acceptable adjuvant can be an oil-in-water emulsion, ISA-206, Quil A, interleukin 12 or a heat shock protein. In further embodiments of these aspects, the variant is a mutant, which can be a fusion protein. The fusion protein can contain the sequence of C. difficile toxins A or B, for example, the N-terminal catalytic domain of TcdA, the N-terminal catalytic domain of TcdB, C-terminal fragment 4 of TcdB, or the C-terminal receptor binding fragment of TcdA. Alternatively, the fusion protein can be a fusion of any one of the proteins BclA1, BclA2, BclA3, Alr, SlpA paralogue, SlpA HMW, CD1021, IunH, and Fe-Mn-SOD, and fragments thereof, or a protein having the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and fragments thereof, with another member of the group of proteins.

In a thirteenth aspect, the present invention provides an isolated nucleic acid encoding a C. difficile spore polypeptide or fragment or variant, which can be BclA1, BclA2, BclA3, Alr, SlpA paralogue, SlpA HMW, CD1021, IunH, Fe-Mn-SOD, or FliD.

In a fourteenth aspect, the present invention provides an isolated nucleic acid encoding a C. difficile spore polypeptide or fragment or variant, where the nucleic acid encodes an amino acid sequence at least 80-95% identical to the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In various embodiments of the thirteenth and fourteenth aspects, the variant is a mutant, which can be a fusion protein. The fusion protein can contain the sequence of C. difficile toxins A or B, for example, the N-terminal catalytic domain of TcdA, the N-terminal catalytic domain of TcdB, C-terminal fragment 4 of TcdB, or the C-terminal receptor binding fragment of TcdA. Alternatively, the fusion protein can be a fusion of any one of the proteins BclA1, BclA2, BclA3, Alr, SlpA paralogue, SlpA HMW, CD1021, IunH, Fe-Mn-SOD, or FliD, and fragments thereof, or a protein having the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and fragments thereof, with another member of the group of proteins.

In yet further embodiments of the thirteenth and fourteenth aspects, the nucleic acids are contained within an expression vector, which can be either a bacterial or mammalian expression vector. Examples of mammalian expression vectors include those that contain the CMV promoter. Other mammalian expression vectors include pcDNA3002Neo or pET32a. Examples of bacterial expression vectors include pET32a. In some embodiments of these aspects, the expression vector can be contained within in a host cell, such as HEK293F, NSO-1, CHO-K1, CHO-S, or PER.C6 in the case of mammalian cell expression, and E. coli, in the case of bacterial expression.

### BRIEF DESCRIPTION OF THE DRAWINGS

FIG. 1A shows a restriction digestion of BclA3-pcDNA3002Neo with Asc I and Hpa I to confirm the presence of BclA3 insert in the plasmid. The expected size of BclA3 removed from the pcDNA3002Neo plasmid is 1.6 kb, and the empty pcDNA3002Neo plasmid is 6.8 kb. (Lane 1: undigested BclA3-pcDNA3002Neo plasmid; Lane 2: digested BclA3-pcDNA3002Neo plasmid).

FIG. 1B shows SDS-PAGE and western blot analysis of purified BclA3 protein. BclA3 transfected supernatant was purified on a HisTRAP Ni column using the Akta Purifier. The eluted protein was loaded on an SDS-PAGE gel (left) in a volume of 15 μL before it was concentrated. (Lane 1: Purified BclA3 protein). The expected size of the protein is 44 kDa. A second gel (right) was run with 8 μg of protein and was transferred to nitrocellulose membrane and probed with antibody against the His-tag of the expressed protein (Lane 1: Purified BclA3 Protein −8 μg).

FIG. 2A shows a restriction digestion of Alr-pcDNA3002Neo with AscI and HpaI to confirm the presence of Alr insert in the plasmid. The expected size of Alr removed from the pcDNA3002Neo plasmid is 1.3 kb and the empty pcDNA3002Neo plasmid is 6.8 kb. (Lane 1: undigested Alr-pcDNA3002Neo plasmid; Lane 2: digested Alr-pcDNA3002Neo plasmid).

FIG. 2B shows SDS-PAGE analysis of purified Alr protein. Alr transfected supernatant was purified on a HisTRAP Ni column using the Akta Purifier. The eluted protein was loaded on an SDS-PAGE gel (left) at 2 μg with and without beta-mercaptoethanol (2ME) (Lane 1: Purified Alr Protein −2 μg; Lane 2: Purified Alr Protein −2 μg+2ME). The expected size of the protein is 45 kDa. A second gel (right) was run with ˜30 μg of protein to exaggerate the difference between the protein +/−2ME (Lane 3: Purified Alr Protein −2 μg; Lane 4: Purified Alr Protein −2 μg+2ME).

FIG. 2C shows western blot analysis of purified Alr protein. Alr transfected supernatant was purified on a HisTRAP Ni column using the Akta Purifier. The eluted protein was loaded on an SDS-PAGE gel at 2 μg with and without beta-mercaptoethanol (2ME) then transferred to nitrocellulose membrane and probed with anti-his antibody (1:3000) (Lane 2: Purified Alr Protein −2 μg; Lane 3: Purified Alr Protein −2 μg+2ME). The expected size of the protein is 45 kDa.

FIG. 3A shows a restriction digestion of SlpA para-pcDNA3002Neo with AscI and HpaI to confirm the presence of SlpA paralogue insert in the plasmid. The expected size of SlpA paralogue removed from the pcDNA3002Neo plasmid is 1.9 kb, and the empty pcDNA3002Neo plasmid is 6.8 kb. (Lane 1: undigested SlpA para-pcDNA3002Neo plasmid; Lane 2: digested SlpA para-pcDNA3002Neo plasmid).

FIG. 3B shows SDS-PAGE and western blot analysis of purified SlpA paralogue. SlpA paralogue transfected supernatant was purified on a HisTRAP Ni column using the Akta Purifier. The eluted protein was loaded on an SDS-PAGE gel (left and middle) at 2 μg with and without beta-mercaptoethanol (2ME). (Lane 1: Purified SlpA paralogue protein—2 μg; Lane 2: Purified SlpA paralogue protein—2 μg+2ME). The expected size of the protein is 84 kDa. In Another gel (right) was run with 2 μg of protein, which was transferred to a nitrocellulose membrane and probed with antibody against the His-tag of the expressed protein (Lane 4: purified SlpA paralogue protein—2 μg).

FIG. 4A shows a restriction digestion of CD1021-pcDNA3002Neo with AscI and HpaI to confirm the presence of CD1021 insert in the plasmid. The expected size of CD1021 removed from the pcDNA3002Neo plasmid is 1.8 kb, and the empty pcDNA3002Neo plasmid is 6.8 kb. (Lane 1: undigested CD1021-pcDNA3002Neo plasmid; Lane 2: digested CD1021-pcDNA3002Neo plasmid).

FIG. 4B shows SDS-PAGE analysis of purified CD1021. CD1021 transfected supernatant was purified on a HisTRAP Ni column using the Akta Purifier. The eluted protein was loaded on an SDS-PAGE gel at 2 μg (Lane 1: Purified CD1021 protein; Lane 2: Purified CD1021 protein +2ME). The expected size of the protein without glycosylation is 65 kDa.

FIG. 4C shows western blot analysis of purified CD1021. Another gel was run with 2 μg of protein, which was transferred to a nitrocellulose membrane and probed with antibody against the His-tag of the expressed protein (Lane 1 on left blot: Purified CD1021 protein +2ME; Lane 1 on right blot: Purified CD1021 protein).

FIG. 5 shows SDS-PAGE analysis of recombinant C. difficile toxin A fragment 4 and toxin B fragment 1 regions and whole Tcd A and B toxins. (A) Toxin A fragment 4 (Lane 1) on a colloidal blue-stained SDS-PAGE gel. The expected size of Toxin A fragment 4 is 114 kDa. (B) Toxin B fragment 1 (Lane 1) on an anti-His probed western immunoblot. The expected size of Toxin B fragment 1 is 82 kDa. (C) Whole Toxin B (Lane 1) and whole Toxin A (Lane 2) on a colloidal blue-stained SDS-PAGE gel. The expected size for Toxin A is 308 kDa and the expected size of Toxin B is 270 kDa.

FIG. 6 shows SDS-PAGE of purified FliD. FliD transfected supernatant was purified on a HisTRAP Ni column using the Akta Purifier. The eluted protein was loaded on an SDS-PAGE gel at 2 μg (Lane 1: Purified FliD Protein +2ME; Lane 2: Purified FIiD Protein). The expected size of the protein without glycosylation is 55 kDa.

FIG. 7 shows the results of ELISA to detect the binding of CD1021 antibodies in mouse sera to isolated C. difficile spores from ATCC 43255.

FIG. 8 shows the results of ELISA to detect binding of FliD antibodies in mouse sera to isolated C. difficile spores from strain ATCC 43255.

FIG. 9 shows the results of ELISA to detect binding of Alr antibodies in mouse sera to isolated C. difficile spores from strain ATCC 43255.

FIG. 10 shows the results of ELISA to detect binding of BclA3 antibodies in mouse sera to isolated C. difficile spores from strain ATCC 43255.

FIG. 11 shows the results of ELISA to detect binding of FliD antibodies in mouse sera to purified C. difficile FLiD protein.

FIG. 12 shows the results of ELISA to detect binding of Alr antibodies in mouse sera to purified C. difficile Alr protein.

FIG. 13 shows the results of ELISA to detect binding of BclA3 antibodies in mouse sera to purified C. difficile BlcA3 protein.

FIG. 14 shows the results of ELISA to detect binding of CD1021 antibodies in mouse sera to purified C. difficile CD1021 protein.

FIG. 15 shows the results of a germination assay to examine the inhibitory effect of anti-spore antibodies on ATCC 43255 spore germination.

FIG. 16 shows a Coomassie blue stain of C. difficile spore antigens.

FIG. 17 shows a Western blot of C. difficile spore antigens probed with a rabbit anti-C. difficile spore pAb.

FIG. 18 shows a Western blot of C. difficile spore antigens probed with sera from Alr immunized mice.

FIG. 19 shows a Western blot of C. difficile spore antigens probed with sera from BclA3 immunized mice.

FIG. 20 shows a Western blot of C. difficile spore antigens probed with sera from CD1021 immunized mice.

FIG. 21 shows a Western blot of C. difficile spore antigens probed with sera from FliD immunized mice.

### DETAILED DESCRIPTION

The present invention generally relates to compositions and methods for the prevention or treatment of bacterial infection by the Gram-positive organism, Clostridium difficile, in a vertebrate subject. Methods for inducing an immune response to Clostridium difficile infection are provided. The methods provide administering a protein or agent to the vertebrate subject in need thereof in an amount effective to reduce, eliminate, or prevent Clostridium difficile bacterial infection or bacterial carriage.

Compositions and methods are provided for inducing an immune response to Clostridium difficile bacteria in a subject comprising administering to the subject a composition comprising an isolated polypeptide, such as Clostridium difficile spore antigens, and an adjuvant in an amount effective to induce the immune response in the subject. The method can be used for the generation of antibodies for use in passive immunization or as a component of a vaccine to prevent infection or relapse from infection by Clostridium difficile.

It is to be understood that this invention is not limited to particular methods, reagents, compounds, compositions or biological systems, which can, of course, vary. It is also to be understood that the terminology used herein is for the purpose of describing particular aspects only, and is not intended to be limiting. As used in this specification and the appended claims, the singular forms “a”, “an” and “the” include plural references unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

“Vertebrate,” “mammal,” “subject,” “mammalian subject,” or “patient” are used interchangeably and refer to mammals such as human patients and non-human primates, as well as experimental animals such as rabbits, rats, and mice, cows, horses, goats, and other animals. Animals include all vertebrates, e.g., mammals and non-mammals, such as mice, sheep, dogs, cows, avian species, ducks, geese, pigs, chickens, amphibians, and reptiles.

The term “adjuvant” refers to an agent which acts in a nonspecific manner to increase an immune response to a particular antigen or combination of antigens, thus, for example, reducing the quantity of antigen necessary in any given composition and/or the frequency of injection necessary to generate an adequate immune response to the antigen of interest. See, e.g., A. C. Allison J. Reticuloendothel. Soc. (1979) 26:619-630. Such adjuvants are described further below. The term “pharmaceutically acceptable adjuvant” refers to an adjuvant that can be safely administered to a subject and is acceptable for pharmaceutical use.

As used herein, “colonization” refers to the presence of Clostridium difficile in the intestinal tract of a mammal.

“Bacterial carriage” is the process by which bacteria such as Clostridium difficile can thrive in a normal subject without causing the subject to get sick. Bacterial carriage is a very complex interaction of the environment, the host and the pathogen. Various factors dictate asymptomatic carriage versus disease. Therefore an aspect of the invention includes treating or preventing bacterial carriage.

“Treating” or “treatment” refers to either (i) the prevention of infection or reinfection, e.g., prophylaxis, or (ii) the reduction or elimination of symptoms of the disease of interest, e.g., therapy. “Treating” or “treatment” can refer to the administration of a composition comprising a polypeptide of interest, e.g., Clostridium difficile spore antigens or antibodies raised against these antigens. Treating a subject with the composition can prevent or reduce the risk of infection and/or induce an immune response to the polypeptide of interest. Treatment can be prophylactic (to prevent or delay the onset of the disease, or to prevent the manifestation of clinical or subclinical symptoms thereof) or therapeutic suppression or alleviation of symptoms after the manifestation of the disease.

“Preventing” or “prevention” refers to prophylactic administration or vaccination with polypeptide or antibody compositions.

“Therapeutically-effective amount” or “an amount effective to reduce or eliminate bacterial infection” or “an effective amount” refers to an amount of polypeptide or antibody that is sufficient to prevent Clostridium difficile bacterial infection or to alleviate (e.g., mitigate, decrease, reduce) at least one of the symptoms associated with Clostridium difficile bacterial infection or to induce an immune response to a Clostridium difficile antigen. It is not necessary that the administration of the composition eliminate the symptoms of Clostridium difficile bacterial infection, as long as the benefits of administration of compound outweigh the detriments. Likewise, the terms “treat” and “treating” in reference to Clostridium difficile bacterial infection, as used herein, are not intended to mean that the subject is necessarily cured of infection or that all clinical signs thereof are eliminated, only that some alleviation or improvement in the condition of the subject is effected by administration of the composition.

As used herein, the term “immune response” refers to the response of immune system cells to external or internal stimuli (e.g., antigen, cell surface receptors, cytokines, chemokines, and other cells) producing biochemical changes in the immune cells that result in immune cell migration, killing of target cells, phagocytosis, production of antibodies, other soluble effectors of the immune response, and the like.

“Protective immunity” or “protective immune response” are intended to mean that the subject mounts an active immune response to a composition, such that upon subsequent exposure to Clostridium difficile bacteria or bacterial challenge, the subject is able to combat the infection. Thus, a protective immune response will generally decrease the incidence of morbidity and mortality from subsequent exposure to Clostridium difficile bacteria among subjects. A protective immune response will also generally decrease colonization by Clostridium difficile bacteria in the subjects.

“Active immune response” refers to an immunogenic response of the subject to an antigen, e.g., Clostridium difficile spore antigens. In particular, this term is intended to mean any level of protection from subsequent exposure to Clostridium difficile bacteria or antigens which is of some benefit in a population of subjects, whether in the form of decreased mortality, decreased symptoms, such as bloating or diarrhea, prevention of relapse, or the reduction of any other detrimental effect of the disease, and the like, regardless of whether the protection is partial or complete. An “active immune response” or “active immunity” is characterized by “participation of host tissues and cells after an encounter with the immunogen. It generally involves differentiation and proliferation of immunocompetent cells in lymphoreticular tissues, which lead to synthesis of antibody or the development cell-mediated reactivity, or both.” Herbert B. Herscowitz, “Immunophysiology: Cell Function and Cellular Interactions in Antibody Formation,” in Immunology: Basic Processes 117 (Joseph A. Bellanti ed., 1985). Alternatively stated, an active immune response is mounted by the host after exposure to immunogens by infection, or as in the present case, by administration of a composition. Active immunity can be contrasted with passive immunity, which is acquired through the “transfer of preformed substances (e.g., antibody, transfer factor, thymic graft, interleukin-2) from an actively immunized host to a non-immune host.” Id.

“Passive immunity” refers generally to the transfer of active humoral immunity in the form of pre-made antibodies from one individual to another. Thus, passive immunity is a form of short-term immunization that can be achieved by the transfer of antibodies, which can be administered in several possible forms, for example, as human or animal blood plasma or serum, as pooled animal or human immunoglobulin for intravenous (IVIG) or intramuscular (IG) use, as high-titer animal or human IVIG or IG from immunized subjects or from donors recovering from a disease, and as monoclonal antibodies. Passive transfer can be used prophylactically for the prevention of disease onset, as well as, in the treatment of several types of acute infection. Typically, immunity derived from passive immunization lasts for only a short period of time, and provides immediate protection, but the body does not develop memory, therefore the patient is at risk of being infected by the same pathogen later.

### Polypeptides

The term “polypeptide” or “peptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “isolated protein,” “isolated polypeptide,” or “isolated peptide” is a protein, polypeptide or peptide that by virtue of its origin or source of derivation (1) is not associated with naturally associated components that accompany it in its native state, (2) is free of other proteins from the same species, (3) is expressed by a cell from a different species, or (4) does not occur in nature. Thus, a peptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The terms “polypeptide”, “protein”, “peptide,” “antigen,” or “antibody” within the meaning of the present invention, includes variants, analogs, orthologs, homologs and derivatives, and fragments thereof that exhibit a biological activity, generally in the context of being able to induce an immune response in a subject, or bind an antigen in the case of an antibody.

The polypeptides of the invention include an amino acid sequence derived from Clostridium difficile spore antigens or fragements thereof, corresponding to the amino acid sequence of a naturally occurring protein or corresponding to variant protein, i.e., the amino acid sequence of the naturally occurring protein in which a small number of amino acids have been substituted, added, or deleted but which retains essentially the same immunological properties. In addition, such derived portion can be further modified by amino acids, especially at the N- and C-terminal ends to allow the polypeptide or fragment to be conformationally constrained and/or to allow coupling to an immunogenic carrier after appropriate chemistry has been carried out. The polypeptides of the present invention encompass functionally active variant polypeptides derived from the amino acid sequence of Clostridium difficile spore antigens in which amino acids have been deleted, inserted, or substituted without essentially detracting from the immunological properties thereof, i.e. such functionally active variant polypeptides retain a substantial peptide biological activity. Typically, such functionally variant polypeptides have an amino acid sequence homologous, preferably highly homologous, to an amino acid sequence such as those in SEQ ID Nos: 1 to 4.

In one embodiment, such functionally active variant polypeptides exhibit at least 60%, 65%, 70%, 75%, 80%, 81%, 82%, 83%, 84%, 85%, 86%, 87%, 88%, 89% 90%, 91%, 92%, 93%, 94%, 95%, 96%, 97%, 98%, or 99% identity to an amino acid sequence selected from the group consisting of SEQ ID Nos: 1 to 4. Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). An alternative algorithm when comparing a sequence of the invention to a database containing a large number of sequences from different organisms is the computer program BLAST, especially blastp or tblastn, using default parameters. See, e.g., Altschul et al., J. Mol. Biol. 215:403-410 (1990); Altschul et al., Nucleic Acids Res. 25:3389-402 (1997).

Functionally active variants comprise naturally occurring functionally active variants such as allelic variants and species variants and non-naturally occurring functionally active variants that can be produced by, for example, mutagenesis techniques or by direct synthesis.

A functionally active variant can exhibit, for example, at least 60%, 65%, 70%, 75%, 80%, 81%, 82%, 83%, 84%, 85%, 86%, 87%, 88%, 89% 90%, 91%, 92%, 93%, 94%, 95%, 96%, 97%, 98%, or 99% identity to an amino acid sequence of a Clostridrium difficile spore antigen disclosed herein, and yet retain a biological activity. Where this comparison requires alignment, the sequences are aligned for maximum homology. The site of variation can occur anywhere in the sequence, as long as the biological activity is substantially similar to the Clostridrium difficile spore antigens disclosed herein, e.g., ability to induce an immune reponse. Guidance concerning how to make phenotypically silent amino acid substitutions is provided in Bowie et al., Science, 247: 1306-1310 (1990), which teaches that there are two main strategies for studying the tolerance of an amino acid sequence to change. The first strategy exploits the tolerance of amino acid substitutions by natural selection during the process of evolution. By comparing amino acid sequences in different species, the amino acid positions which have been conserved between species can be identified. These conserved amino acids are likely important for protein function. In contrast, the amino acid positions in which substitutions have been tolerated by natural selection indicate positions which are not critical for protein function. Thus, positions tolerating amino acid substitution can be modified while still maintaining specific immunogenic activity of the modified polypeptide.

The second strategy uses genetic engineering to introduce amino acid changes at specific positions of a cloned gene to identify regions critical for protein function. For example, site-directed mutagenesis or alanine-scanning mutagenesis can be used (Cunningham et al., Science, 244: 1081-1085 (1989)). The resulting variant polypeptides can then be tested for specific biological activity.

According to Bowie et al., these two strategies have revealed that proteins are surprisingly tolerant of amino acid substitutions. The authors further indicate which amino acid changes are likely to be permissive at certain amino acid positions in the protein. For example, the most buried or interior (within the tertiary structure of the protein) amino acid residues require nonpolar side chains, whereas few features of surface or exterior side chains are generally conserved.

Methods of introducing a mutation into amino acids of a protein is well known to those skilled in the art. See, e. g., Ausubel (ed.), Current Protocols in Molecular Biology, John Wiley and Sons, Inc. (1994); T. Maniatis, E. F. Fritsch and J. Sambrook, Molecular Cloning: A Laboratory Manual, Cold Spring Harbor laboratory, Cold Spring Harbor, N.Y. (1989)).

Mutations can also be introduced using commercially available kits such as “QuikChange Site-Directed Mutagenesis Kit” (Stratagene) or directly by peptide synthesis. The generation of a functionally active variant to an peptide by replacing an amino acid which does not significantly influence the function of said peptide can be accomplished by one skilled in the art.

A type of amino acid substitution that may be made in the polypeptides of the invention is a conservative amino acid substitution. A “conservative amino acid substitution” is one in which an amino acid residue is substituted by another amino acid residue having a side chain R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See e.g. Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al., Science 256:1443-45 (1992). A “moderately conservative” replacement is any change having a nonnegative value in the PAM250 log-likelihood matrix.

A functionally active variant can also be isolated using a hybridization technique. Briefly, DNA having a high homology to the whole or part of a nucleic acid sequence encoding the peptide, polypeptide or protein of interest, e.g. Clostridium difficile spore antigens, is used to prepare a functionally active peptide. Therefore, a polypeptide of the invention also includes entities which are functionally equivalent and which are encoded by a nucleic acid molecule which hybridizes with a nucleic acid encoding any one of the Clostridium difficile spore antigens or a complement thereof. One of skill in the art can easily determine nucleic acid sequences that encode peptides of the invention using readily available codon tables. As such, these nucleic acid sequences are not presented herein.

Nucleic acid molecules encoding a functionally active variant can also be isolated by a gene amplification method such as PCR using a portion of a nucleic acid molecule DNA encoding a peptide, polypeptide, protein, antigen, or antibody of interest, e.g. Clostridium difficile spore antigens, as the probe.

For the purpose of the present invention, it should be considered that several polypeptides, proteins, peptides, antigens, or antibodies of the invention may be used in combination. All types of possible combinations can be envisioned. For example, an antigen comprising more than one polypeptide, preferably selected from the Clostridium difficile spore antigens disclosed herein, could be used. In some embodiments, the antigen could include one or more spore antigens in combination with an antigen derived from a vegetative cell, such as toxins A or B. The same sequence can be used in several copies on the same polypeptide molecule, or wherein peptides of different amino acid sequences are used on the same polypeptide molecule; the different peptides or copies can be directly fused to each other or spaced by appropriate linkers. As used herein the term “multimerized (poly)peptide” refers to both types of combination wherein polypeptides of either different or the same amino acid sequence are present on a single polypeptide molecule. From 2 to about 20 identical and/or different peptides can be thus present on a single multimerized polypeptide molecule.

In one embodiment of the invention, a peptide, polypeptide, protein, or antigen of the invention is derived from a natural source and isolated from a bacterial source. A peptide, polypeptide, protein, or antigen of the invention can thus be isolated from sources using standard protein purification techniques.

Alternatively, peptides, polypeptides and proteins of the invention can be synthesized chemically or produced using recombinant DNA techniques. For example, a peptide, polypeptide, or protein of the invention can be synthesized by solid phase procedures well known in the art. Suitable syntheses may be performed by utilising “T-boc” or “F-moc” procedures. Cyclic peptides can be synthesised by the solid phase procedure employing the well-known “F-moc” procedure and polyamide resin in the fully automated apparatus. Alternatively, those skilled in the art will know the necessary laboratory procedures to perform the process manually. Techniques and procedures for solid phase synthesis are described in ‘Solid Phase Peptide Synthesis: A Practical Approach’ by E. Atherton and R. C. Sheppard, published by IRL at Oxford University Press (1989) and ‘Methods in Molecular Biology, Vol. 35: Peptide Synthesis Protocols (ed. M. W. Pennington and B. M. Dunn), chapter 7, pp 91-171 by D. Andreau et al.

Alternatively, a polynucleotide encoding a peptide, polypeptide or protein of the invention can be introduced into an expression vector that can be expressed in a suitable expression system using techniques well known in the art, followed by isolation or purification of the expressed peptide, polypeptide, or protein of interest. A variety of bacterial, yeast, plant, mammalian, and insect expression systems are available in the art and any such expression system can be used. Optionally, a polynucleotide encoding a peptide, polypeptide or protein of the invention can be translated in a cell-free translation system.

Nucleic acid sequences corresponding to Clostridium difficile spore antigens can also be used to design oligonucleotide probes and used to screen genomic or cDNA libraries for genes from other Clostridium difficile variants or even other bacterial species. The basic strategies for preparing oligonucleotide probes and DNA libraries, as well as their screening by nucleic acid hybridization, are well known to those of ordinary skill in the art. See, e.g., DNA Cloning: Vol. I, supra; Nucleic Acid Hybridization, supra; Oligonucleotide Synthesis, supra; Sambrook et al., supra. Once a clone from the screened library has been identified by positive hybridization, it can be confirmed by restriction enzyme analysis and DNA sequencing that the particular library insert contains a Clostridium difficile gene, or a homolog thereof. The genes can then be further isolated using standard techniques and, if desired, PCR approaches or restriction enzymes employed to delete portions of the full-length sequence.

Alternatively, DNA sequences encoding the proteins of interest can be prepared synthetically rather than cloned. The DNA sequences can be designed with the appropriate codons for the particular amino acid sequence. In general, one will select preferred codons for the intended host if the sequence will be used for expression. The complete sequence is assembled from overlapping oligonucleotides prepared by standard methods and assembled into a complete coding sequence. See, e.g., Edge (1981) Nature 292: 756; Nambair et al. (1984) Science 223: 1299; Jay et al. (1984) J. Biol. Chem. 259: 6311.

Once coding sequences for the desired proteins have been prepared or isolated, they can be cloned into any suitable vector or replicon. Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, Sambrook et al., supra; DNA Cloning, supra; B. Perbal, supra. The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence can or can not contain a signal peptide or leader sequence. Leader sequences can be removed by the host in post-translational processing. See, e.g., U.S. Pat. Nos. 4,431,739; 4,425,437; 4,338,397. Examples of vectors include pET32a(+) and pcDNA3002Neo.

Other regulatory sequences can also be desirable which allow for regulation of expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements can also be present in the vector, for example, enhancer sequences.

The control sequences and other regulatory sequences can be ligated to the coding sequence prior to insertion into a vector, such as the cloning vectors described above. Alternatively, the coding sequence can be cloned directly into an expression vector which already contains the control sequences and an appropriate restriction site.

In some cases it can be necessary to modify the coding sequence so that it can be attached to the control sequences with the appropriate orientation; i.e., to maintain the proper reading frame. It can also be desirable to produce mutants or analogs of the protein. Mutants or analogs can be prepared by the deletion of a portion of the sequence encoding the protein, by insertion of a sequence, and/or by substitution of one or more nucleotides within the sequence. Techniques for modifying nucleotide sequences, such as site-directed mutagenesis, are described in, e.g., Sambrook et al., supra; DNA Cloning, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Madin-Darby bovine kidney (“MDBK”) cells, HEK293F cells, NSO-1 cells, as well as others. Similarly, bacterial hosts such as E. coli, Bacillus subtilis, and Streptococcus spp., will find use with the present expression constructs. Yeast hosts useful in the present invention include, but are not limited to, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, but are not limited to, Aedes aegypti, Autographa californica, Bombyx mori, Drosophila melanogaster, Spodoptera fmgiperda, and Trichoplusia ni.

Expression vectors having a polynucleotide of interest, e.g. Clostridium difficile spore antigens, can also be vectors normally used by one of skill in the art for DNA vaccination of a host in need thereof. DNA vaccination can be used in any manner, e.g., for the first host antigenic challenge and/or for a boost challenge with the antigen of interest. General characteristics of DNA vaccination and the associated techniques are well known in the art. Appropriate dosages of DNA vectors can also be readily determined using well-defined techniques for measuring whether an immune response has been generated to the antigen(s) of interest and/or whether protection has been established in the host to bacterial challenge.

Depending on the expression system and host selected, the proteins of the present invention are produced by culturing host cells transformed by an expression vector described above under conditions whereby the protein of interest is expressed. The protein is then isolated from the host cells and purified. The selection of the appropriate growth conditions and recovery methods are within the skill of the art.

Clostridium difficile spore antigen protein sequences can also be produced by chemical synthesis such as solid phase peptide synthesis, using known amino acid sequences or amino acid sequences derived from the DNA sequence of the genes of interest. Such methods are known to those skilled in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis. Chemical synthesis of peptides can be preferable if a small fragment of the antigen in question is capable of raising an immunological response in the subject of interest.

Polypeptides of the invention can also comprise those that arise as a result of the existence of multiple genes, alternative transcription events, alternative RNA splicing events, and alternative translational and postranslational events. A polypeptide can be expressed in systems, e.g. cultured cells, which result in substantially the same postranslational modifications present as when the peptide is expressed in a native cell, or in systems that result in the alteration or omission of postranslational modifications, e.g. glycosylation or cleavage, present when expressed in a native cell.

A peptide, polypeptide, protein, or antigen of the invention can be produced as a fusion protein that contains other distinct amino acid sequences that are not part of the Clostridium difficile spore antigen sequences disclosed herein, such as amino acid linkers or signal sequences or immunogenic carriers, as well as ligands useful in protein purification, such as glutathione-S-transferase, histidine tag, and staphylococcal protein A. More than one polypeptide of the invention can be present in a fusion protein. The heterologous polypeptide can be fused, for example, to the N-terminus or C-terminus of the peptide, polypeptide or protein of the invention. A peptide, polypeptide, protein, or antigen of the invention can also be produced as fusion proteins comprising homologous amino acid sequences. Examples of fusion proteins useful in the practice of the present invention include, but are not limited to, fusions of the Clostridium difficile spore antigens described herein with portions of Clostridium difficile toxins A or B, e.g., the N-terminal catalytic domain of Tcd A, the N-terminal catalytic domain of Tcd B, or the C-terminal fragment 4 of TcdB. The Clostridium difficile spore antigens, or fragements thereof, can also be fused to each other to form fusion proteins suitable for use in the present invention.

### Clostridium Spore Proteins

Any of a variety of Clostridium difficile spore proteins may be used in the practice of the present invention. Such spore proteins can be identified by searching known Clostridium difficile sequences, including the complete genome sequences of a number strains that have recently been sequenced. Further examples of spore proteins useful in the practice of the present invention are also described in the literature. See, e.g., Henriques and Moran, Annual Rev. Microbiol., 61: 555-88 (2007). Representative examples of Clostridium difficile spore proteins include those described below.

BcIA proteins, including BclA1, BclA2, and BclA3, are collagen-like proteins which are involved in the formation of the exosporium of C. difficile spores. The exosporium surrounds the spore coat and contributes to spore resistance. Targets such as surface exposed exosporium proteins are good potential target for therapy. For example, the BclA proteins have orthologues in Bacillus anthracis, and it has been shown that immunization with BclA has shown protection in animals from B. anthracis spore colonization by inhibiting germination. Representative examples of C. difficile BcIA sequences that can be used in the practice of the present invention include, but are not limited, to proteins with the NCBI accession numbers: FN545816 (regions 402547-404145; 3689444-3691084; and 3807430-3809466 for BclA1, A2, and A3, respectively).

Alr (Alanine racemase) protein in C. difficile is an exosporium enzyme involved in a quorum-sensing type mechanism that links germination to the number of spores present in a nutrient-limited medium. An orthologous protein is also present in Bacillus species, where the protein has been shown to be present in the late stages of sporulation and to be necessary to suppress premature germination thereby enhancing survival of the bacteria. Representative examples of C. difficile Alr sequences that can be used in the practice of the present invention include, but are not limited, to proteins with the NCBI accession number: FN545816 (region 3936313-3937470).

SlpA protein encodes the S-layer which is the predominant surface antigen on the spore. The SlpA protein has been shown to induce a strong serum IgG response in patients (See Kelleher D. et al., J. Med. Micro., 55:69-83 (2006)). The protein is divided into an N-terminal (LMW) portion and a C-terminal (HMW) portion. The SlpA HMW protein is highly conserved and therefore attractive as a target.

SlpA paralogue protein refers to a large family of open reading frames (paralogues) in C. difficile strain 630 that are related to the amino acid sequence of the high-MW SlpA subunit. This amino acid sequence is 45% homologous (including conservative replacements) to two cell wall-bound proteins of Bacillus subtilis, an N-acetylmuramoyl-L-alanine amidase (CWLB/LytC) and its enhancer (CWBA/LytB). The sequence homology has a functional correlate, as the C. difficile high-MW SLP subunit shows amidase activity. By analogy with B. subtilis, it has been suggested that the homology domain mediates anchoring to the cell wall and therefore identifies a class of cell wall components. Consistent with this, many slpA paralogs encode a typical signal sequence, indicating that they are secreted or membrane bound. Of the 29 slpA paralogs identified so far, 12 map in a densely arranged cluster surrounding slpA and are all transcribed in the same direction, suggesting the possibility of coordinated regulation and related functions. It has been shown that the six slpA-like genes immediately 3′ of slpA (ORFs 2 to 7) are transcribed during vegetative growth. COG2247 a putative cell wall-binding domain. Representative examples of C. difficile slpA sequences that can be used in the practice of the present invention include, but are not limited, to proteins with the NCBI accession numbers: FN545816 (region 3157304-3159175; 3162172-3164448). Shown below in Example 3 is COG2247, a putative cell wall-binding domain.

CD1021 (CotH) protein is a hypothetical protein found on the C. difficile spore to which antibodies have been made. Because this protein is surface exposed, it would make a good target for therapy. Representative examples of C. difficile CD1021 sequences that can be used in the practice of the present invention include, but are not limited, to proteins with the NCBI accession number: AM180355 (region 1191725-1193632).

IunH encodes an inosine hydrolase, an enzyme found in the exosporium of Bacillus anthracis, for which C. difficile has an orthologue. This enzyme has been suggested to have a role in the initiation of spore germination. A representative example of a C. difficile IunH sequence that can be used in the practice of the present invention includes, but is not limited, to a protein with the NCBI accession number: FN545816 (region 1866580-1867548).

Fe-Mn-SOD or superoxide dismutase (SOD) is a class of enzymes that catalyze the dismutation of superoxide into oxygen and hydrogen peroxide and are therefore an important anti-oxidant defense in cells. Many bacteria contain a form of the enzyme with iron and manganese. A representative example of a C. difficile Fe-Mn-SOD sequence that can be used in the practice of the present invention includes, but is not limited, to proteins with the NCBI accession number: NC_{—}013316 (region 1802293-1802997).

The fliD gene encodes the flagellar cap protein (FliD) of C. difficile. This protein has been shown to have adhesive properties in vitro and in vivo, and in particular, has been shown to have a role in attachment to mucus. It has been shown that antibody levels against FliD were significantly higher in a control group versus a group of patients with CDAD, suggesting that the protein is able to induce an immune response that could play a role in host defense mechanisms. A separate study showed that the protein was present in 15 out of 17 clinical isolates tested, suggesting that it is present in most strains. The same study also showed that out of the 17 patients with different clinical isolates, 15 had antibody against FliD. Representative examples of C. difficile FliD sequences that can be used in the practice of the present invention include, but are not limited, to proteins with the NCBI accession numbers: Q9AHP4, AF297024, AF297025, AF297026, AF297027, and AF297028.

Table 1 provides exemplary amino acid sequences of Clostridium difficile spore antigen proteins that can be used in the practice of the present invention. It is understood that variants and fragments of the exemplary sequences provided below are also encompassed by the present invention.

TABLE 1

| | Accession | |
|---|---|---|
| SEQ | Number And | |
| ID | Protein Name | |
| NO | And Description | Amino acid Sequence |
| 1 | FN545816 | MACPGFLWALVISTCLEFSMAMRKIILYLNDDTFISKKYPDK |
| | (region: | NFSNLDYCLIGSKCSNSFVKEKLITFFKVRIPDILKDKSILKAE |
| | 402547- | LFIHIDSNKNHIFKEKVDIEIKRISEYYNLRTITWNDRVSMENI |
| | 404145) | RGYLPIGISDTSNYICLNITGTIKAWAMNKYPNYGLALSLNYP |
| | | YQIFEFTSSRDCNKPYILVTFEDRIIDNCYPKCECLPIRITGPMG |
| | Bc1A1 | PRGATGSIGPMGATGPTGATG |
| 2 | FN545816 | MACPGFLWALVISTCLEFSMAMSDISGPSLYQDVGPTGPTGA |
| | (region: | TGPTGPTGPRGATGATGANGITGPTGNTGATGANGITGPTGN |
| | 3689444- | MGATGANGTTGSTGPTGNTGATGANGITGPTGATGATGAN |
| | 3691084) | GITGPTGNKGATGANGITGPTGATGATGANGITGPTGNTGAT |
| | | GANGATGLTGATGATGANGITGPTGATGATGANGVTGATG |
| | Bc1A2 | PTGNTGATGPTGSIGATGANGVTGATGPIGATGPTGAVGAT |
| | | GPDGLVGPTGPTGPTGATGANGLVGPTGPTGATGANGLVGP |
| | | TGATGATGVAGAIGPTGAVGATGPTGADGAVGPTGATGAT |
| | | GANGATGPTGAVGATGANGVAGPIGPTGPTGANGVAGATG |
| | | ATGATGANGATGPTGAVGATGANGVAGPIGPTGPTGANGTT |
| | | GATGATGATGANGATGPTGATGATGVLAANNAQFTVSSSSL |
| | | GNNTLVTFNSSFINGTN |
| 3 | FN545816 | MACPGFLWALVISTCLEFSMAMSRNKYFGPFDDNDYNNGY |
| | (region: | DKYDDCNNGRDDYNSCDCHHCCPPSCVGPTGPMGPRGRTG |
| | 3807430- | PTGPTGPTGPGVGGTGPTGPTGPTGPTGNTGNTGATGLRGPT |
| | 3809466) | GATGGTGPTGATGAIGFGVTGPTGPTGPTGATGATGADGVT |
| | | GPTGPTGATGADGITGPTGATGATGFGVTGPTGPTGATGVG |
| | Bc1A3 | VTGATGLIGPTGATGTPGATGPTGAIGATGIGITGPTGATGAT |
| | | GADGATGVTGPTGPTGATGADGVTGPTGATGATGIGITGPT |
| | | GATGATGIGITGATGLIGPTGATGATGATGPTGVTGATGAAG |
| | | LIGPTGATGVTGADGATGATGATGATGPTGADGLVGPTGAT |
| | | GATGADGLVGPTGPTGATGVGITGATGATGATGPTGADGLV |
| | | GPTGATGATGADGVAGPTGATGATGNTGADGATGPTGATG |
| | | PTGADGLVGPTGATGATGLAGATGATGPIGATGPTGADGAT |
| | | GATGATGPTGADGLVGPTGATGATGATGPTGP |
| 4 | FN545816 | MACPGFLWALVISTCLEFSMAMQKITVPTWAEINLDNLRFNL |
| | (region: | NNIKNLLEEDIKICGVIKADAYGHGAVEVAKLLEKEKVDYL |
| | 3936313- | AVARTAEGIELRQNGITLPILNLGYTPDEAFEDSIKNKITMTV |
| | 3937470) | YSLETAQKINEIAKSLGEKACVHVKIDSGMTRIGFQPNEESVQ |
| | | EIIELNKLEYIDLEGMFTHFATADEVSKEYTYKQANNYKFMS |
| | A1r | DKLDEAGVKIAIKHVSNSAAIMDCPDLRLNMVRAGIILYGHY |
| | | PSDDVFKDRLELRPAMKLKSKIGHIKQVEPGVGISYGLKYTT |
| | | TGKETIATVPIGYADGFTRIQKNPKVLIKGEVFDVVGRICMD |
| | | QIMVRIDKDIDIKVGDEVILFGEGEVTAERIAKDLGTINYEVL |
| | | CMISRRVDRVYMENNELVQINSYLLK |
| 5 | FN545816 | MACPGFLWALVISTCLEFSMAAETTQVKKETITKKEATELVS |
| | (region: | KVRDLMSQKYTGGSQVGQPIYEIKVGETLSKLKIITNIDELEK |
| | 3157304- | LVNALGENKELIVTITDKGHITNSANEVVAEATEKYENSADL |
| | 3159175) | SAEANSITEKAKTETNGIYKVADVKASYDSAKDKLVITLRDK |
| | | TDTVT SKTIEIGIGDEKIDLTANPVDSTGTNLDPSTEGFRVNKI |
| | S1pA | VKLGVAGAKNIDDVQLAEITIKNSDLNTVSPQDLYDGYRLT |
| | paralogue | VKGNMVANGTSKSISDISSKDSETGKYKFTIKYTDASGKAIEL |
| | | TVESTNEKDLKDAKAALEGNSKVKLIAGDDRYATAVAIAKQ |
| | | TKYTDNIVIVNSNKLVDGLAATPLAQSKKAPILLASDNEIPKV |
| | | TLDYIKDIIKKSPSAKIYIVGGESAVSNTAKKQLESVTKNVER |
| | | LAGDDRHMTSVAVAKAMGSFKDAFVVGAKGEADAMSIAA |
| | | KAAELKAPIIVNGWNDLSADAIKLMDGKEIGIVGGSNNVSSQ |
| | | IENQLADVDKDRKVQRVEGETRHDTNAKVIETYYGKLDKLY |
| | | IAKDGYGNNGMLVDALAAGPLAAGKGPILLAKADITDSQRN |
| | | ALSKKLNLGAEVTQIGNGVELTVIQKIAKILGW |
| 6 | FN545816 | MGKTAQDLAKKYVFNKTDLNTLYRVLNGDEADTNRLVEEV |
| | (region: | SGKYQVVLYPEGKRVTTKSAAKASIADENSPVKLTLKSDKK |
| | 3162172- | KDLKDYVDDLRTYNNGYSNAIEVAGEDRIETAIALSQKYYN |
| | 3164448) | SDDENAIFRDSVDNVVLVGGNAIVDGLVASPLASEKKAPLLL |
| | | TSKDKLDSSVKAEIKRVMNIKSTTGINTSKKVYLAGGVNSIS |
| | S1pA HMW | KEVENELKDMGLKVTRLAGDDRYETSLKIADEVGLDNDKA |
| | | FVVGGTGLADAMSIAPVASQLRNANGKMDLADGDATPIVV |
| | | VDGKAKTINDDVKDFLDDSQVDIIGGENSVSKDVENAIDDAT |
| | | GKSPDRYSGDDRQATNAKVIKESSYYQDNLNNDKKVVNFF |
| | | VAKDGSTKEDQLVDALAAAPVAANFGVTLNSDGKPVDKDG |
| | | KVLTGSDNDKNKLVSPAPIVLATDSLSSDQSVSISKVLDKDN |
| | | GENLVQVGKGIATSVINKLKDLLSMLEGT |
| 7 | AM180355 | MACPGFLWALVISTCLEFSMATSSNKSVDLYSDVYIEKYFNR |
| | (region: | DKVMEVNIEIDESDLKDMNENAIKEEFKVAKVTVDGDTYGN |
| | 1191725- | VGIRTKGNSSLISVANSDSDRYSYKINFDKYNTSQSMEGLTQ |
| | 1193632) | LNLNNCYSDPSYMREFLTYSICEEMGLATPEFAYAKVSINGE |
| | | YHGLYLAVEGLKESYLENNFGNVTGDLYKSDEGSSLQYKGD |
| | CD1021 | DPESYSNLIVESDKKTADWSKITKLLKSLDTGEDIEKYLDVD |
| | (CotH) | SVLKNIAINTALLNLDSYQGSFAHNYYLYEQDGVFSMLPWD |
| | | FNMSFGGFSGFGGGSQSIAIDEPTTGNLEDRPLISSLLKNETY |
| | | KTKYHKYLEEIVTKYLDSDYLENMTTKLHDMIASYVKEDPT |
| | | AFYTYEEFEKNITSSIEDSSDNKGFGNKGFDNNNSNNSDSNN |
| | | NSNSENKRSGNQSDEKEVNAELTSSVVKANTDNETKNKTTN |
| | | DSESKNNTDKDKSGNDNNQKLEGPMGKGGKSIPGVLEVAE |
| | | DMSKTIKSQLSGETSSTKQNSGDESSSGIKGSEKFDEDMSGM |
| | | PEPPEGMDGKMPPGMGNMDKGDMNGKNGNMNMDRNQDN |
| | | PREAGGFGNRGGGSVSKTTTYFK |
| 8 | FN545816 | MEKRKVIIDCDPGIDDSLAILLALNSPELEVIGITTCCGNVPAN |
| | (region: | IGAENALKTLQMCSSLNIPVYIGEEAPLKRKLVTAQDTHGED |
| | 1866580- | GIGENFYQKVVGAKAKNGAVDFIINTLHNHEKVSIIALAPLT |
| | 1867548) | NIAKALIKDKKAFENLDEFVSMGGAFRIHGNCSPVAEFNYW |
| | | VDPHGADYVYKNLSKKIHMVGLDVTRKIVLTPNIIEFINRLD |
| | IunH | KKMAKYITEITRFYIDFHWEQEGIIGCVINDPLAVAYFIDRSIC |
| | | KGFESYVEVVEDGIAMGQSIVDSFNFYKKNPNAIVLNEVDEK |
| | | KFMYMFLKRLFKGYEDIIDSVEGVI |
| 9 | NC_013316 | MKKKILIPVIMSLFIISQCITSFAFTPENNKFKVKPLPYAYDAL |
| | (region: | EPYIDKETMKLHHDKHYQAYVDKLNAALEKYPELYNYSLC |
| | 1802293- | ELLQNLDSLPKDIATTVRNNAGGAYNHKFFFDIMTPEKTIPSE |
| | 1802997) | SLKEAIDRDFGSFEKFKQEFQKSALDVFGSGWAWLVATKDG |
| | | KLSIMTTPNQDSPVSKNLTPIIGLDVWEHAYYLKYQNRRNEY |
| | Fe-Mn-SOD | IDNWFNVVNWNGALENYKNLKSQD |
| 10 | Q9AHP4 | MSSISPVRVTGLSGNFDMEGIIEASMIRDKEKVDKAKQEQQI |
| | | VKWKQEIYRNVIQESKDLYDKYLSVNSPNSIVSEKAYSSTRIT |
| | | SSDESIIVAKGSAGAEKINYQFAVSQMAEPAKFTIKLNSSEPIV |
| | F1iD | RQFPPNASGASSLTIGDVNIPISEQDTTSTIVSKINSLCADNDIK |
| | | ASYSEMTGELIISRKQTGSSSDINLKVIGNDNLAQQIANDNGI |
| | | TFANDASGNKVASVYGKNLEADVTDEHGRVTHISKEQNSFN |
| | | IDNIDYNVNSKGTAKLTSVTDTEEAVKNMQAFVDDYNKLM |
| | | DKVYGLVTTKKPKDYPPLTDAQKEDMTTEEIEKWEKKAKE |
| | | GILRNDDELRGFVEDIQSAFFGDGKNIIALRKLGINESENYNK |
| | | KGQISFNADTFSKALIDDSDKVYKTLAGYSSNYDDKGMFEK |
| | | LKDIVYEYSGSSTSKLPKKAGIEKTASASENVYSKQIAEQERN |
| | | ISRLVEKMNDKEKRLYAKYSALESLLNQYSSQMNYFSQAQG |
| | | N |
| 11 | Bc1A3 with | AATMACPGFLWALVISTCLEFSMAMSRNKYFGPFDDNDYN |
| | Kozak, | NGYDKYDDCNNGRDDYNSCDCHHCCPPSCVGPTGPMGPRG |
| | HAVT20 | RTGPTGPTGPTGPGVGGTGPTGPTGPTGPTGNTGNTGATGLR |
| | leader, and | GPTGATGGTGPTGATGAIGFGVTGPTGPTGPTGATGATGAD |
| | His-tag | GVTGPTGPTGATGADGITGPTGATGATGFGVTGPTGPTGAT |
| | sequences | GVGVTGATGLIGPTGATGTPGATGPTGAIGAGIGITGPTGAT |
| | | GATGADGATGVTGPTGPTGATGADGVTGPTGATGATGIGIT |
| | | GPTGATGATGIGITGATGLIGPTGATGATGATGPTGVTGATG |
| | | AAGLIGPTGATGVTGADGATGATGATGATGPTGADGLVGPT |
| | | GATGATGADGLVGPTGPTGATGVGITGATGATGATGPTGAD |
| | | GLVGPTGATGATGADGVAGPTGATGATGNTGADGATGPTG |
| | | ATGPTGADGLVGPTGATGATGLAGATGATGPIGATGPTGAD |
| | | GATGATGATGPTGADGLVGPTGATGATGATGPTGPHHHHH |
| | | H |
| 12 | A1r with | IRRAATMACPGFLWALVISTCLEFSMAMQKITVPTWAEINLD |
| | Kozak, | NLRFNLNNIKNLLEEDIKICGVIKADAYGHGAVEVAKLLEKE |
| | HAVT20 | KVDYLAVARTAEGIELRQNGITLPILNLGYTPDEAFEDSIKNK |
| | leader, and | ITMTVYSLETAQKINEIAKSLGEKACVHVKIDSGMTRIGFQPN |
| | His-tag | EESVQEIIELNKLEYIDLEGMFTHFATADEVSKEYTYKQANN |
| | sequences | YKFMSDKLDEAGVKIAIKHVSNSAAIMDCPDLRLNMVRAGII |
| | | LYGHYPSDDVFKDRLELRPAMKLKSKIGHIKQVEPGVGISYG |
| | | LKYTTTGKETIATVPIGYADGFTRIQKNPKVLIKGEVFDVVG |
| | | RICMDQIMVRIDKDIDIKVGDEVILFGEGEVTAERIAKDLGTI |
| | | NYEVLCMISRRVDRVYMENNELVQINSYLLKHHHHHH |
| 13 | S1pA | AATMACPGFLWALVISTCLEFSMAAETTQVKKETITKKEATE |
| | Paralogue | LVSKVRDLMSQKYTGGSQVGQPIYEIKVGETLSKLKIITNIDE |
| | with Kozak, | LEKLVNALGENKELIVTITDKGHITNSANEVVAEATEKYENS |
| | HAVT20 | ADLSAEANSITEKAKTETNGIYKVADVKASYDSAKDKLVITL |
| | leader, and | RDKTDTVTSKTIEIGIGDEKIDLTANPVDSTGTNLDPSTEGFR |
| | His-tag | VNKIVKLGVAGAKNIDDVQLAEITIKNSDLNTVSPQDLYDGY |
| | sequences | RLTVKGNMVANGTSKSISDISSKDSETGKYKFTIKYTDASGK |
| | | AIELTVESTNEKDLKDAKAALEGNSKVKLIAGDDRYATAVAI |
| | | AKQTKYTDNIVIVNSNKLVDGLAATPLAQSKKAPILLASDNE |
| | | IPKVTLDYIKDIIKKSPSAKIYIVGGESAVSNTAKKQLESVTKN |
| | | VERLAGDDRHMTSVAVAKAMGSFKDAFVVGAKGEADAMS |
| | | IAAKAAELKAPIIVNGWNDLSADAIKLMDGKEIGIVGGSNNV |
| | | SSQIENQLADVDKDRKVQRVEGETRHDTNAKVIETYYGKLD |
| | | KLYIAKDGYGNNGMLVDALAAGPLAAGKGPILLAKADITDS |
| | | QRNALSKKLNLGAEVTQIGNGVELTVIQKIAKILGWHHHHH |
| | | H |
| 14 | CD1021 | IRRAATMACPGFLWALVISTCLEFSMATSSNKSVDLYSDVYI |
| | with Kozak, | EKYFNRDKVMEVNIEIDESDLKDMNENAIKEEFKVAKVTVD |
| | HAVT20 | GDTYGNVGIRTKGNSSLISVANSDSDRYSYKINFDKYNTSQS |
| | leader, and | MEGLTQLNLNNCYSDPSYMREFLTYSICEEMGLATPEFAYA |
| | His-tag | KVSINGEYHGLYLAVEGLKESYLENNFGNVTGDLYKSDEGS |
| | sequences | SLQYKGDDPESYSNLIVESDKKTADWSKITKLLKSLDTGEDI |
| | | EKYLDVDSVLKNIAINTALLNLDSYQGSFAHNYYLYEQDGV |
| | | FSMLPWDFNMSFGGFSGFGGGSQSIAIDEPTTGNLEDRPLISS |
| | | LLKNETYKTKYHKYLEEIVTKYLDSDYLENMTTKLHDMIAS |
| | | YVKEDPTAFYTYEEFEKNITSSIEDSSDNKGFGNKGFDNNNS |
| | | NNSDSNNNSNSENKRSGNQSDEKEVNAELTSSVVKANTDNE |
| | | TKNKTTNDSESKNNTDKDKSGNDNNQKLEGPMGKGGKSIP |
| | | GVLEVAEDMSKTIKSQLSGETSSTKQNSGDESSSGIKGSEKFD |
| | | EDMSGMPEPPEGMDGKMPPGMGNMDKGDMNGKNGNMN |
| | | MDRNQDNPREAGGFGNRGGGSVSKTTTYFKHHHHHH |
| 15 | F1iD with | IRRAATMACPGFLWALVISTCLEFSMAIRDKEKVDKAKQEQ |
| | Kozak, | QIVKWKQEIYRNVIQESKDLYDKYLSVNSPNSIVSEKAYSST |
| | HAVT20 | RITSSDESIIVAKGSAGAEKINYQFAVSQMAEPAKFTIKLNSSE |
| | leader, and | PIVRQFPPNASGASSLTIGDVNIPISEQDTTSTIVSKINSLCADN |
| | His tag | DIKASYSEMTGELIISRKQTGSSSDINLKVIGNDNLAQQIAND |
| | sequences | NGITFANDASGNKVASVYGKNLEADVTDEHGRVTHISKEQN |
| | | SFNIDNIDYNVNSKGTAKLTSVTDTEEAVKNMQAFVDDYNK |
| | | LMDKVYGLVTTKKPKDYPPLTDAQKEDMTTEEIEKWEKKA |
| | | KEGILRNDDELRGFVEDIQSAFFGDGKNIIALRKLGINESENY |
| | | NKKGQISFNADTFSKALIDDSDKVYKTLAGYSSNYDDKGMF |
| | | EKLKDIVYEYSGSSTSKLPKKAGIEKTASASENVYSKQIAEQE |
| | | RNISRLVEKMNDKEKRLYAKYSALESLLNQYSSQMNYFSQA |
| | | QGNHHHHHH |

Table 2 provides nucleic acid sequences encoding the proteins of Table 1.

TABLE 2

| | Accession | |
|---|---|---|
| SEQ | Number | |
| ID | And Gene | |
| NO | Name | Nucleotide Sequence |
| 16 | FN545816 | ATGAGAAAAATTATACTTTATTTAAATGATGATACTTTTAT |
| | (region: | ATCTAAAAAATATCCAGATAAAAACTTTAGTAATTTAGATT |
| | 402547- | ATTGCTTAATAGGAAGTAAATGTTCAAATAGTTTTGTAAAA |
| | 404145) | GAAAAGTTGATTACTTTTTTTTAAGTGAGAATACCAGATAT |
| | | ATTAAAAGACAAAAGTATATTAAAAGCAGAGTTATTTATT |
| | Bc1A1 | CATATTGATTCAAATAAGAATCATATTTTTAAAGAAAAAGT |
| | | AGATATTGAAATTAAAAGAATAAGTGAATATTATAATTTA |
| | | CGAACTATAACATGGAATGATAGAGTGTCTATGGAAAATA |
| | | TCAGGGGATATTTACCAATTGGGATAAGTGATACATCCAA |
| | | CTATATTTGTTTAAATATTACGGGAACTATAAAAGCATGGG |
| | | CAATGAATAAATATCCTAATTATGGGTTAGCTTTATCTTTA |
| | | AATTACCCTTATCAGATTTTTGAATTTACATCTAGTAGGGA |
| | | TTGTAACAAACCGTATATACTTGTAACATTTGAAGATAGAA |
| | | TTATAGATAATTGTTATCCTAAATGTGAGTGTCTTCCAATT |
| | | AGAATTACAGGTCCAATGGGACCAAGAGGAGCGACAGGA |
| | | AGTATAGGACCAATGGGAGCAACAGGTCCAACAGGAGCA |
| | | ACAGGCAATTCCTCTCAGCCAATTGCTAACTTCCTCGTAAA |
| | | TGCACCATCTCCACAAACACTAAATAATGGAAATGCTATA |
| | | ACAGGTTGGTAAACAATAATAGGAAATAGTTCAAGTATAA |
| | | CAGTAGATGCAAATGGTACGTTTACAGTACAAGAAAATGG |
| | | TGTGTATTATATATCAGTTTCAGTAGCATTACAACCAGGTT |
| | | CATCAAGTATAAATCAATATTCTTTTGCTATCCTATTCCCA |
| | | ATTTTAGGAGGAAAAGATTTGGCAGGGCTTACTACTGAGC |
| | | CAGGAGGCGGAGGAGTACTTTCTGGATATTTTGCTGGTTTT |
| | | TTATTTGGGGGGACTACTTTTACAATAAATAATTTTTCATCT |
| | | ACAACAGTAGGGATACGAAATGGGCAATCAGCAGGAACTG |
| | | CGGCTACTTTGACGATATTTAGAATAGCTGATACTGTTATG |
| | | ACTTAAAACGTGTCTAAAATAATCTTAAAAACTATTTAGGT |
| | | TTTATTTAAATGACAAAAGTATTTTTATATATTGAGTTTTAC |
| | | CTATTTTAGAATGAATAAAATAACAATAATAATAAAATAT |
| | | ATTCATAAAAATTTTAAATTTATGGATTTTTATTTAACTTTA |
| | | TTATCAATATATGTATAATAAAAAACTGTCTCAAATATAGA |
| | | TTTGAGACAGTTTTCGTTATTTAAAAATTTTATATTATTTAA |
| | | AATTTTTGATTGCAGTAGTTAAATTAGGGACTAATTGTTTT |
| | | TTTCTTGATACAACACCTGGTGCAAATGTACCTTGAACACC |
| | | TTCAACATCAAATGCGTTAGCAATTAAGCTATCTTCTGCTT |
| | | TATAGATTAAATAAGAACCTTCTTTGATTATATCTGTAACC |
| | | GCAAGAATTAGTTTGTCATAATCAGTCGAATTTATATAAGA |
| | | TAAAAACTCATCTTTTTTAGCAAATATAGAGTCTATGTCTA |
| | | AGGTAAATACTTGTCCAATACCAACTCTATGTCCACTCATA |
| | | TTAAATTCTTTAAAATCCATATTTACTATTTCTTCTATAGTA |
| | | TATTCATCTAAAGAAGTACCGCATTTAAACATATCCATAGC |
| | | GTATTTTTCCATGTCTACTTTTGCTATTTTACTTAATTCTTCA |
| | | CAAGCTTTCTTATCCATATCAGTTGTTGTTGGAGACTTAAA |
| | | TAATAATGTATCTGATAATATAGCAGATAAAAGAAGCCCA |
| | | GCTATTTCATAAGGTATCTCAACATTGTTTTCTTTGTACATT |
| | | TGATAAATTATAGTACTATTGCATCCAACAGGCATAACTCT |
| | | AAATGACATAGGAACATCAGTAGAAATACCACCAAGTTTA |
| | | TGATGGTCAATTATTTCAACTATGTTTGCTTGTTCAATTCCA |
| | | TCAGCACTTTGAGCATATTCGTTATGGTCAACTAAAACAAC |
| | | ATTCTTTTTAGATGGGTTTAATAGATGACCTTTTGAAACTA |
| | | AACCTAAAAACTTATTATCATCATCT |

| | | |
|---|---|---|
| 17 | FN545816 | ATGAGTGATATTTCAGGTCCAAGTTTATATCAAGATGTAGG |
| | (region: | TCCAACAGGGCCAACAGGTGCTACTGGTCCAACAGGACCG |
| | 3689444..3 | ACGGGGCCTAGAGGCGCAACCGGAGCGACCGGAGCAAAT |
| | 691084) | GGAATAACAGGACCAACAGGAAATACGGGAGCAACCGGG |
| | | GCGAATGGAATAACAGGACCAACAGGAAATATGGGAGCG |
| | Bc1A2 | ACTGGAGCAAATGGAACAACAGGTTCTACAGGACCAACAG |
| | | GAAATACAGGAGCGACTGGAGCAAATGGAATAACAGGTC |
| | | AACAGGAGCAACAGGAGCAACGGGAGCAAATGGAATAAC |
| | | AGGTCCAACCGGAAACAAGGGAGCAACGGGAGCGAATGG |
| | | GATAACAGGTCCAACAGGAGCAACAGGAGCAACGGGAGC |
| | | AAATGGAATAACAGGTCCAACAGGAAATACAGGAGCAAC |
| | | GGGAGCAAATGGTGCAACCGGACTAACCGGAGCAACTGGG |
| | | GCAACGGGAGCGAATGGGATAACAGGTCCAACAGGAGCA |
| | | ACAGGAGCAACGGGAGCAAATGGAGTAACAGGTGCTACA |
| | | GGCCCAACAGGAAATACAGGAGCAACAGGTCCAACCGGA |
| | | AGTATAGGAGCAACGGGAGCAAATGGAGTAACAGGTGCC |
| | | ACAGGTCCAATAGGAGCAACAGGTCCAACCGGAGCAGTAG |
| | | GAGCAACAGGTCCAGATGGTTTGGTAGGTCCAACAGGCCC |
| | | AACAGGCCCAACCGGAGCAACCGGAGCAAATGGTTTGGTA |
| | | GGTCCAACAGGCCCAACCGGAGCAACCGGAGCAAATGGTT |
| | | TGGTAGGTCCAACAGGAGCGACCGGAGCAACAGGAGTAGC |
| | | TGGGGCAATAGGTCCAACCGGAGCAGTAGGAGCGACAGGC |
| | | CCAACGGGAGCAGATGGAGCAGTAGGTCCAACCGGAGCA |
| | | ACCGGAGCAACAGGGGCAAATGGAGCAACAGGCCCAACG |
| | | GGAGCAGTAGGAGCAACTGGAGCGAATGGAGTAGCAGGT |
| | | CCAATAGGTCCAACAGGTCCAACCGGAGCAAATGGAGTAG |
| | | CAGGAGCAACAGGAGCGACCGGAGCAACAGGGGCAAATG |
| | | GAGCAACAGGCCCAACAGGAGCAGTAGGAGCAACGGGAG |
| | | CAAATGGAGTAGCAGGTCCAATAGGTCCAACAGGACCAAC |
| | | AGGAGCAAATGGAACGACCGGAGCAACAGGGGCGACCGG |
| | | AGCAACGGGAGCAAATGGAGCAACAGGTCCAACAGGAGC |
| | | GACCGGAGCAACAGGAGTGTTAGCAGCAAACAATGCACAA |
| | | TTTACAGTATCTTCTTCAAGTTTAGGGAATAATACATTAGT |
| | | GACATTTAATTCATCATTTATAAATGGAACTAATATAACTT |
| | | TTCCAACAAGTAGTACTATAAATCTTGCAGTTGGAGGGATA |
| | | TACAATGTATCTTTCGGTATACGTGCCATACTTTCACTTGC |
| | | AGGATTTATGTCAATTACTACTAACTTTAATGGAGTAGCCC |
| | | AAAATAACTTTATTGCAAAAGCAGTAAATACGCTTACTTCA |
| | | TCAGATGTAAGTGTAAGTTTAAGCTTTTTAGTTGATGCTAG |
| | | AGCAGCAGCTGTTACTTTAAGCTTTACATTTGGTTCAGGCA |
| | | CGACAGGTACTTCTCCAGCTGGGTATGTATCAGTTTATAGA |
| | | ATACAATAG |
| 18 | FN545816 | ATGAGTAGAAATAAATATTTTGGACCATTTGATGATAATGA |
| | (region: | TTACAACAATGGCTATGATAAATATGATGATTGTAATAATG |
| | 3807430- | GTCGTGATGATTATAATAGCTGTGATTGCCATCATTGCTGT |
| | 3809466) | CCACCATCATGTGTAGGTCCAACAGGCCCAATGGGTCCAA |
| | | GAGGTAGAACCGGCCCAACAGGACCAACGGGTCCAACAG |
| | Bc1A3 | GTCCAGGAGTAGGGGGAACAGGCCCAACAGGACCAACG |
| | | GTCCGACTGGCCCAACAGGAAATACAGGGAATACAGGAGC |
| | | AACAGGATTAAGAGGTCCAACAGGAGCAACAGGGGGAAC |
| | | AGGCCCAACAGGAGCGACAGGAGCTATAGGGTTTGGAGTA |
| | | ACAGGCCCAACAGGCCCAACAGGCCCAACAGGAGCGACA |
| | | GGAGCAACAGGAGCAGATGGAGTAACAGGTCCAACAGGT |
| | | CCAACGGGAGCAACAGGAGCAGATGGAATAACAGGTCCA |
| | | ACAGGAGCAACAGGGGCAACAGGATTTGGAGTAACAGGTC |
| | | CAACAGGCCCAACAGGAGCAACAGGAGTAGGAGTAACAG |
| | | GAGAACAGGATTAATAGGTCCAACAGGAGCGACAGGAA |
| | | CACCTGGAGCAACAGGTCCAACAGGGGCAATAGGAGCAAC |
| | | AGGAATAGGAATAACAGGTCCAACAGGAGCAACAGGAGC |
| | | AACAGGGGCAGATGGAGCAACAGGAGTAACAGGCCCAAC |
| | | AGGCCCAACAGGGGCAACAGGAGCAGATGGAGTAACAGG |
| | | CCCAACAGGAGCAACAGGAGCAACAGGAATAGGAATAAC |
| | | AGGCCCAACAGGGGCAACAGGAGCAACAGGAATAGGAAT |
| | | AACAGGAGCAACAGGGTTAATAGGTCCAACCGGAGCAACC |
| | | GGAGCAACCGGAGCAACAGGCCCAACAGGAGTAACAGGG |
| | | GCAACAGGAGCAGCAGGACTAATAGGACCAACCGGGGCA |
| | | ACAGGAGTAACCGGAGCAGATGGAGCAACAGGAGCGACA |
| | | GGGGCAACCGGAGCAACAGGTCCAACAGGAGCAGATGGA |
| | | TTAGTAGGTCCAACAGGAGCAACAGGGGCAACAGGAGCA |
| | | GATGGATTAGTAGGCCCAACAGGTCCAACAGGGGCAACCG |
| | | GAGTAGGAATAACTGGAGCAACCGGAGCAACAGGAGCGA |
| | | CAGGTCCAACAGGAGCAGATGGATTAGTAGGTCCAACCGG |
| | | AGCGACGGGAGCAACAGGAGCAGATGGAGTAGCAGGTCC |
| | | AACCGGAGCAACAGGGGCAACAGGAAATACAGGAGCAGA |
| | | TGGAGCAACAGGTCCAACAGGGGCAACAGGTCCAACAGG |
| | | AGCAGACGGATTAGTAGGTCCAACAGGAGCAACCGGAGCA |
| | | ACAGGATTAGCAGGAGCAACCGGAGCAACAGGCCCAATA |
| | | GGAGCAACAGGTCCAACAGGAGCAGATGGAGCAACAGGG |
| | | GCAACCGGAGCAACAGGTCCAACAGGGGCAGATGGATTAG |
| | | TAGGTCCAACCGGAGCAACGGGAGCAACAGGGGCAACAG |
| | | GTCCAACAGGCCCAACAGGTGCTAGTGCAATAATACCTTTT |
| | | GCATCAGGTATACCACTATCACTTACAACTATAGCTGGAGG |
| | | ATTAGTAGGTACACCAGGTTTTGTTGGCTTTGGTAGTTCAG |
| | | CTCCAGGATTAAGTATAGTTGGTGGAGTAATAGACCTTACA |
| | | AACGCAGCAGGGACATTGACTAACTTTGCATTTTCAATGCC |
| | | AAGAGATGGAACAATAACATCTATTTCAGCATACTTCAGT |
| | | ACAACAGCAGCACTTTCACTTGTTGGTTCAACAATTACAAT |
| | | TACAGCAACACTTTACCAATCTACTGCACCAAATAACTCAT |
| | | TTACAGCTGTACCAGGAGCGACAGTTACACTAGCTCCACC |
| | | ACTTACAGGTATATTATCAGTTGGTTCAATTTCTAGTGGAA |
| | | TTGTAACAGGATTAAATATAGCAGCAACAGCAGAAACTCG |
| | | ATTCTTACTAGTATTTACTGCAACAGCTTCAGGTCTTTCATT |
| | | AGTTAATACTGTAGCAGGATATGCAAGTGCAGGAATTGCA |
| | | ATAAATTAG |
| 19 | FN545816 | ATGCAAAAAATAACAGTGCCTACATGGGCAGAGATAAATC |
| | (region: | TAGATAACTTAAGATTTAACTTAAATAATATTAAAAATTTA |
| | 3936313- | TTAGAAGAAGATATTAAGATTTGTGGAGTAATAAAAGCTG |
| | 3937470) | ATGCATATGGACATGGTGCAGTAGAAGTTGCAAAATTGCT |
| | | AGAAAAAGAAAAAGTAGATTACTTAGCAGTAGCAAGAACT |
| | A1r | GCTGAAGGAATTGAACTTAGACAAAATGGCATAACACTTC |
| | | CTATTTTGAACTTGGGATATACTCCAGACGAAGCTTTTGAA |
| | | GATTCTATAAAAAATAAAATAACTATGACAGTTTATTCTTT |
| | | AGAAACAGCACAAAAGATAAATGAAATTGCAAAATCTTTA |
| | | GGAGAAAAAGCCTGTGTTCATGTTAAAATAGACTCAGGGA |
| | | TGACTAGAATAGGTTTCCAACCTAATGAGGAGTCAGTACA |
| | | GGAAATAATAGAATTAAATAAATTAGAATATATCGATTTA |
| | | GAAGGTATGTTTACTCATTTTGCTACAGCTGATGAAGTAAG |
| | | TAAAGAGTACACTTATAAACAAGCTAATAATTATAAATTTA |
| | | TGTCTGATAAATTAGATGAGGCTGGTGTAAAAATAGCTAT |
| | | AAAACATGTATCAAACAGTGCAGCTATTATGGATTGCCCTG |
| | | ATTTAAGATTAAATATGGTAAGAGCAGGAATAATATTATA |
| | | TGGTCATTATCCATCTGATGATGTATTTAAAGATAGATTAG |
| | | AATTAAGACCAGCCATGAAATTAAAATCAAAAATCGGACA |
| | | TATAAAACAAGTTGAACCAGGTGTAGGAATAAGTTATGGA |
| | | CTAAAATACACAACTACAGGTAAAGAAACAATAGCTACAG |
| | | TTCCAATAGGATACGCAGATGGATTTACTAGAATCCAAAA |
| | | AAATCCAAAGGTTCTTATTAAGGGAGAAGTGTTTGATGTA |
| | | GTTGGTAGAATATGTATGGATCAAATAATGGTTAGAATTG |
| | | ACAAAGATATAGACATAAAAGTTGGAGATGAGGTTATACT |
| | | ATTTGGAGAAGGCGAAGTTACAGCTGAGCGTATAGCTAAA |
| | | GACTTAGGAACTATAAACTATGAAGTGTTATGTATGATATC |
| | | AAGAAGAGTTGACCGTGTTTATATGGAAAATAATGAGCTT |
| | | GTACAAATAAACAGTTATTTGCTAAAATAA |
| 20 | FN545816 | ATGAATAAAAAAAATCTTTCTGTAATTATGGCTGCTGCAAT |
| | (region: | GATAAGTACATCAGTAGCTCCAGTTTTTGCTGCAGAAACTA |
| | 3157304- | CACAGGTAAAAAAAGAAACAATAACTAAGAAAGAAGCTA |
| | 3159175) | CAGAACTAGTTTCGAAAGTTAGAGATTTAATGTCTCAAAA |
| | | GTATACTGGTGGTTCTCAAGTTGGACAACCAATATATGAAA |
| | S1pA | TAAAAGTTGGCGAGACTTTATCAAAATTAAAAATAATAAC |
| | paralogue | TAATATAGATGAATTAGAGAAATTAGTAAATGCTTTGGGA |
| | | GAAAATAAAGAACTTATTGTAACTATAACAGATAAAGGGC |
| | | ATATAACAAATAGTGCAAATGAAGTAGTTGCAGAAGCAAC |
| | | TGAAAAATATGAAAATTCAGCAGACCTTTCCGCTGAGGCT |
| | | AATTCTATAACAGAAAAAGCTAAAACTGAAACTAATGGAA |
| | | TTTATAAAGTTGCAGATGTAAAAGCTTCATATGATAGTGCT |
| | | AAAGATAAGTTAGTTATAACTTTAAGAGATAAAACAGACA |
| | | CAGTAACTTCTAAAACTATAGAGATAGGTATTGGTGATGA |
| | | AAAAATTGATTTAACAGCAAATCCAGTTGATTCAACGGGA |
| | | ACAAACTTAGACCCTTCTACAGAAGGATTTAGAGTAAATA |
| | | AAATCGTTAAACTAGGTGTAGCAGGAGCTAAAAATATTGA |
| | | TGATGTCCAATTAGCTGAAATAACTATAAAAAATAGTGAC |
| | | CTAAATACAGTTTCACCACAAGATTTATATGATGGATATAG |
| | | ATTAACTGTTAAAGGTAATATGGTAGCAAATGGAACATCA |
| | | AAGTCAATTAGTGATATTTCATCAAAAGATTCAGAAACAG |
| | | GAAAGTATAAATTTACTATTAAGTATACTGATGCATCTGGA |
| | | AAAGCAATAGAGCTTACTGTAGAAAGTACTAATGAAAAAG |
| | | ATTTAAAAGATGCCAAAGCTGCATTAGAAGGTAATTCAAA |
| | | GGTTAAATTGATAGCTGGAGATGATAGATATGCAACTGCA |
| | | GT GGCTATAGCAAAACAAACAAAATATACTGACAATATAG |
| | | TTATAGTTAATTCAAATAAACTAGTTGATGGATTAGCAGCT |
| | | ACACCACTTGCTCAATCTAAAAAAGCACCTATATTATTAGC |
| | | ATCCGATAATGAAATACCAAAAGTAACTTTAGATTATATA |
| | | AAAGATATAATTAAGAAAAGCCCATCAGCTAAAATATATA |
| | | TAGTAGGTGGAGAATCAGCAGTATCAAATACAGCTAAAAA |
| | | GCAATTAGAATCAGTAACTAAGAATGTTGAAAGACTAGCT |
| | | GGAGATGATAGACATATGACTTCTGTAGCAGTAGCAAAAG |
| | | CTATGGGGTCTTTTAAAGATGCATTTGTAGTAGGTGCGAAA |
| | | GGGGAGGCTGATGCTATGAGTATAGCTGCCAAAGCTGCTG |
| | | AACTTAAGGCTCCTATAATAGTAAATGGCTGGAATGATCTT |
| | | TCAGCAGACGCTATCAAATTGATGGATGGAAAAGAGATTG |
| | | GTATAGTTGGTGGTTCTAACAATGTATCTAGTCAAATTGAA |
| | | AATCAACTTGCTGATGTTGATAAAGATAGAAAAGTTCAAA |
| | | GAGTTGAAGGAGAAACAAGACACGATACTAATGCTAAGGT |
| | | TATAGAAACATATTATGGAAAATTAGATAAACTATATATA |
| | | GCAAAAGATGGATATGGAAATAATGGTATGCTAGTAGATG |
| | | CATTGGCAGCAGGACCTCTAGCAGCAGGTAAAGGTCCAAT |
| | | ACTTCTAGCTAAAGCTGATATAACAGACTCACAAAGGAAT |
| | | GCACTTAGTAAAAAATTAAATCTTGGTGCAGAAGTAACTC |
| | | AAATAGGTAATGGAGTTGAATTGACAGTAATACAAAAGAT |
| | | AGCTAAAATACTAGGTTGGTAA |
| 21 | FN545816 | ATGAATAAGAAGAATATAGCAATAGCTATGTCAGGATTAA |
| | (region: | CAGTATTAGCTTCTGCAGCACCTGTGTTTGCAGCAGAAGAT |
| | 3162172- | ATGTCGAAAGTTGAGACTGGTGATCAAGGATATACAGTAG |
| | 3164448) | TACAGAGCAAGTATAAGAAAGCAGTTGAACAATTACAAAA |
| | | AGGGTTATTAGATGGAAGTATAACAGAGATTAAAATTTTCT |
| | S1pA HMW | TTGAGGGAACTTTAGCATCTACTATAAAAGTAGGAGCTGA |
| | | GCTTAGTGCAGAAGATGCAAGTAAATTATTGTTTACACAA |
| | | GTAGATAATAAATTAGACAATTTAGGTGATGGGGATTATG |
| | | TAGATTTCTTAATAAGCTCTCCAGCAGAGGGAGATAAAGT |
| | | AACTACAAGTAAACTTGTTGCATTAAAAAATTTAACAGGT |
| | | GGAACTAGTGCAATAAAAGTAGCTACAAGTAGTATTATTG |
| | | GTGAAGTCGAAAATGCTGGTACTCCGGGAGCAAAAAATAC |
| | | AGCTCCAAGTAGTGCTGCAGTTATGTCTATGTCAGATGTAT |
| | | TTGATACAGCTTTTACAGATTCAACTGAAACTGCTGTGAAA |
| | | CTTACTATAAAAGATGCTATGAAAACTAAAAAGTTTGGTTT |
| | | AGTTGATGGAACTACTTATTCAACAGGTCTTCAATTTGCAG |
| | | ATGGAAAAACAGAAAAAATTGTTAAATTAGGAGATAGTGA |
| | | TACTATAAATTTAGCCAAAGAATTAATAATAACACCTGCA |
| | | AGTGCAAATGATCAAGCTGCGACTATTGAGTTTGCTAAACC |
| | | AACAACACAATCTGGAAGCCCAGTAATAACTAAACTTAGA |
| | | ATATTGAATGCAAAAGAAGAGACAATAGATATTGATGCTA |
| | | GTTCTAGTAAAACAGCACAAGATTTAGCTAAAAAATATGT |
| | | ATTTAATAAAACAGATTTAAATACTCTTTACAGAGTATTAA |
| | | ATGGGGATGAAGCAGATACTAATAGATTAGTAGAAGAAGT |
| | | TAGTGGAAAATATCAAGTGGTTCTTTATCCAGAAGGAAAA |
| | | AGAGTTACAACTAAGAGTGCTGCAAAGGCTTCAATTGCTG |
| | | ATGAAAATTCACCAGTTAAATTAACTCTTAAGTCAGATAAG |
| | | AAGAAAGACTTAAAAGATTATGTGGATGATTTAAGAACAT |
| | | ATAATAATGGATATTCAAATGCTATAGAAGTAGCAGGAGA |
| | | AGATAGAATAGAAACTGCAATAGCATTAAGTCAAAAATAT |
| | | TATAACTCTGATGATGAAAATGCTATATTTAGAGATTCAGT |
| | | TGATAATGTAGTATTGGTTGGAGGAAATGCAATAGTTGAT |
| | | GGACTTGTAGCTTCTCCTTTAGCTTCTGAAAAGAAAGCTCC |
| | | TTTATTATTAACTTCAAAAGATAAATTAGATTCAAGCGTAA |
| | | AAGCTGAAATAAAGAGAGTTATGAATATAAAGAGTACAAC |
| | | AGGTATAAATACTTCAAAGAAAGTTTATTTAGCTGGTGGA |
| | | GTTAATTCTATATCTAAAGAAGTAGAAAATGAATTAAAAG |
| | | ATATGGGACTTAAAGTTACAAGATTAGCAGGAGATGATAG |
| | | ATATGAAACTTCTCTAAAAATAGCTGATGAAGTAGGTCTTG |
| | | ATAATGATAAAGCATTTGTAGTTGGAGGAACAGGATTAGC |
| | | AGATGCCATGAGTATAGCTCCAGTTGCATCTCAATTAAGAA |
| | | ATGCTAATGGTAAAATGGATTTAGCTGATGGTGATGCTACA |
| | | CCAATAGTAGTTGTAGATGGAAAAGCTAAAACTATAAATG |
| | | ATGATGTAAAAGATTTCTTAGATGATTCACAAGTTGATATA |
| | | ATAGGTGGAGAAAACAGTGTATCTAAAGATGTTGAAAATG |
| | | CAATAGATGATGCTACAGGTAAATCTCCAGATAGATATAG |
| | | TGGAGATGATAGACAAGCAACTAATGCAAAAGTTATAAAA |
| | | GAATCTTCTTATTATCAAGATAACTTAAATAATGATAAAAA |
| | | AGTAGTTAATTTCTTTGTAGCTAAAGATGGTTCTACTAAAG |
| | | AAGATCAATTAGTTGATGCTTTAGCAGCAGCTCCAGTTGCA |
| | | GCAAACTTTGGTGTAACTCTTAATTCTGATGGTAAGCCAGT |
| | | AGATAAAGATGGTAAAGTATTAACTGGTTCTGATAATGAT |
| | | AAAAATAAATTAGTATCTCCAGCACCTATAGTATTAGCTAC |
| | | TGATTCTTTATCTTCAGATCAAAGTGTATCTATAAGTAAAG |
| | | TTCTTGATAAAGATAATGGAGAAAACTTAGTTCAAGTTGGT |
| | | AAAGGTATAGCTACTTCAGTTATAAATAAATTAAAAGATTT |
| | | ATTAAGTATGTAA |
| 22 | AM180355 | ATGAAAGATAAAAAATTTACCCTTCTTATCTCGATTATGAT |
| | (region: | TGTATTTTTATGTGCTGTAGTTGGAGTTTATAGTACATCTAG |
| | 1191725- | CAACAAAAGTGTTGATTTATATAGTGATGTATATATTGAAA |
| | 1193632) | AATATTTTAACAGAGACAAGGTTATGGAAGTTAATATAGA |
| | | GATAGATGAAAGTGACTTGAAGGATATGAATGAAAATGCT |
| | CD1021 | ATAAAAGAAGAATTTAAGGTTGCAAAAGTAACTGTAGATG |
| | (CotH) | GAGATACATATGGAAACGTAGGTATAAGAACTAAAGGAAA |
| | | TTCAAGTCTTATATCTGTAGCAAATAGTGATAGTGATAGAT |
| | | ACAGCTATAAGATTAATTTTGATAAGTATAATACTAGTCAA |
| | | AGTATGGAAGGGCTTACTCAATTAAATCTTAATAACTGTTA |
| | | CTCTGACCCATCTTATATGAGAGAGTTTTTAACATATAGTA |
| | | TTTGCGAGGAAATGGGATTAGCGACTCCAGAATTTGCATAT |
| | | GCTAAAGTCTCTATAAATGGCGAATATCATGGTTTGTATTT |
| | | GGCAGTAGAAGGATTAAAAGAGTCTTATCTTGAAAATAAT |
| | | TTTGGTAATGTAACTGGAGACTTATATAAGTCAGATGAAG |
| | | GAAGCTCGTTGCAATATAAAGGAGATGACCCAGAAAGTTA |
| | | CTCAAACTTAATCGTTGAAAGTGATAAAAAGACAGCTGAT |
| | | TGGTCTAAAATTACAAAACTATTAAAATCTTTGGATACAGG |
| | | TGAAGATATTGAAAAATATCTTGATGTAGATTCTGTCCTTA |
| | | AAAATATAGCAATAAATACAGCTTTATTAAACCTTGATAGC |
| | | TATCAAGGGAGTTTTGCCCATAACTATTATTTATATGAGCA |
| | | AGATGGAGTATTTTCTATGTTACCATGGGATTTTAATATGT |
| | | CATTTGGTGGATTTAGTGGTTTTGGTGGAGGTAGTCAATCT |
| | | ATAGCAATTGATGAACCTACGACAGGTAATTTAGAAGATA |
| | | GACCTCTCATATCCTCGTTATTAAAAAATGAGACATACAAA |
| | | ACAAAATACCATAAATATCTGGAAGAGATAGTAACAAAAT |
| | | ACCTAGATTCAGACTATTTAGAGAATATGACAACAAAATT |
| | | GCATGACATGATAGCATCATATGTAAAAGAAGACCCAACA |
| | | GCATTTTATACTTATGAAGAATTTGAAAAAAATATAACATC |
| | | TTCAATTGAAGATTCTAGTGATAATAAGGGATTTGGTAATA |
| | | AAGGGTTTGACAACAATAACTCTAATAACAGTGATTCTAAT |
| | | AATAATTCTAATAGTGAAAATAAGCGCTCTGGAAATCAAA |
| | | GTGATGAAAAAGAAGTTAATGCTGAATTAACATCAAGCGT |
| | | AGTCAAAGCTAATACAGATAATGAAACTAAAAATAAAACT |
| | | ACAAATGATAGTGAAAGTAAGAATAATACAGATAAAGATA |
| | | AAAGTGGAAATGATAATAATCAAAAGCTAGAAGGTCCTAT |
| | | GGGTAAAGGAGGTAAGTCAATACCAGGGGTTTTGGAAGTT |
| | | GCAGAAGATATGAGTAAAACTATAAAATCTCAATTAAGTG |
| | | GAGAAACTTCTTCGACAAAGCAAAACTCTGGTGATGAAAG |
| | | TTCAAGTGGAATTAAAGGTAGTGAAAAGTTTGATGAGGAT |
| | | ATGAGTGGTATGCCAGAACCACCTGAGGGAATGGATGGTA |
| | | AAATGCCACCAGGAATGGGTAATATGGATAAGGGAGATAT |
| | | GAATGGTAAAAATGGCAATATGAATATGGATAGAAATCAA |
| | | GATAATCCAAGAGAAGCTGGAGGTTTTGGCAATAGAGGAG |
| | | GAGGCTCTGTGAGTAAAACAACAACATACTTCAAATTAAT |
| | | TTTAGGTGGAGCTTCAATGATAATAATGTCGATTATGTTAG |
| | | TTGGTGTATCAAGGGTAAAGAGAAGAAGATTTATAAAGTC |
| | | AAAATAA |
| 23 | FN545816 | ATGGAAAAGAGAAAAGTAATAATTGATTGTGACCCAGGAA |
| | (region: | TTGATGATTCTTTGGCAATTCTTCTGGCTTTAAACTCACC |
| | 1866580- | AGAGCTAGAAGTAATTGGAATTACCACATGTTGTGGAAAT |
| | 1867548) | GTTCCAGCAAATATAGGTGCAGAAAATGCACTAAAAACAC |
| | | TTCAAATGTGTTCTTCACTAAATATTCCAGTATATATAGGA |
| | IunH | GAAGAAGCACCACTAAAAAGAAAACTTGTAACAGCTCAAG |
| | | ATACACATGGAGAAGATGGTATTGGAGAAAACTTTTATCA |
| | | AAAGGTTGTAGGAGCTAAAGCAAAAAATGGAGCAGTGGAT |
| | | TTTATAATAAATACTTTACATAATCATGAAAAAGTATCAAT |
| | | AATAGCACTTGCACCACTTACAAATATAGCTAAAGCACTTA |
| | | TTAAAGATAAGAAAGCATTTGAAAATCTCGATGAGTTTGT |
| | | ATCTATGGGAGGAGCATTTAGGATTCATGGAAATTGCTCTC |
| | | CAGTAGCAGAGTTTAATTATTGGGTAGACCCACATGGAGC |
| | | AGATTATGTTTACAAGAATTTATCTAAAAAAATCCACATGG |
| | | TAGGTTTAGATGTAACTAGAAAAATTGTACTTACTCCTAAT |
| | | ATTATTGAGTTTATAAATAGACTTGATAAGAAGATGGCAA |
| | | AGTATATAACTGAAATAACTAGATTTTATATTGATTTCCAT |
| | | TGGGAACAGGAAGGAATAATTGGCTGTGTGATAAATGACC |
| | | CTCTAGCAGTAGCGTACTTTATAGACAGAAGTATATGTAAA |
| | | GGATTTGAATCATATGTAGAAGTTGTAGAAGATGGAATAG |
| | | CTATGGGTCAGTCTATAGTGGATTCTTTCAATTTCTATAAA |
| | | AAAAATCCTAATGCAATTGTTCTAAATGAAGTTGATGAGA |
| | | AGAAATTTATGTACATGTTTTTAAAGAGGCTTTTTAAAGGT |
| | | TATGAAGACATTATAGACTCTGTGGAAGGAGTGATATAG |
| 24 | NC_013316 | ATGAAGAAAAAAATATTAATACCAGTTATTATGTCTTTATT |
| | (region: | TATAATCTCACAGTGCATAACTTCATTTGCTTTTACACCTG |
| | 1802293- | AAAATAACAAATTTAAGGTTAAACCATTACCTTATGCATAT |
| | 1802997) | GATGCACTTGAACCTTATATAGATAAAGAAACAATGAAAC |
| | | TGCATCATGATAAGCATTATCAAGCTTATGTTGATAAATTA |
| | Fe-Mn- | AATGCTGCTCTTGAAAAATATCCTGAGCTTTATAATTATTC |
| | SOD | TTTATGTGAATTATTGCAAAATTTAGATTCTTTACCTAAAG |
| | | ATATTGCTACAACTGTAAGAAATAATGCAGGTGGAGCTTA |
| | | TAATCATAAATTCTTTTTTGATATAATGACGCCAGAAAAAA |
| | | CCATACCTTCTGAATCTTTAAAAGAAGCTATTGATAGAGAC |
| | | TTTGGTTCTTTTGAAAAATTTAAGCAAGAGTTCCAAAAATC |
| | | TGCTTTAGATGTCTTTGGTTCTGGTTGGGCTTGGCTTGTAGC |
| | | TACTAAAGATGGGAAATTATCTATTATGACTACTCCAAATC |
| | | AGGATAGCCCTGTAAGTAAAAACCTAACTCCTATAATAGG |
| | | ACTTGATGTTTGGGAGCATGCTTACTATTTAAAATATCAAA |
| | | ATAGAAGAAATGAATACATTGACAACTGGTTTAATGTAGT |
| | | AAATTGGAATGGTGCTTTAGAAAATTACAAAAATTTAAAA |
| | | TCTCAAGATTAA |
| 25 | Q9AHP4 | ATGTCAAGTATAAGTCCAGTAAGAGTTACAGGTCTTTCAGG |
| | | AAATTTTGATATGGAAGGCATAATCGAAGCTAGTATGATT |
| | F1iD | AGAGACAAGGAAAAAGTTGATAAAGCAAAACAAGAACAA |
| | | CAAATCGTTAAATGGAAGCAAGAAATATATAGAAATGTTA |
| | | TACAAGAATCAAAAGATCTTTATGATAAATATCTAAGCGT |
| | | AAATTCTCCTAATAGTATAGTAAGTGAAAAAGCATACTCTT |
| | | CTACAAGAATAACCAGTTCTGATGAAAGTATTATAGTAGC |
| | | AAAAGGCTCAGCTGGTGCAGAAAAAATAAATTATCAATTT |
| | | GCAGTTTCTCAAATGGCTGAACCAGCAAAATTTACTATTAA |
| | | ATTAAATTCAAGTGAACCTATTGTTCGACAGTTCCCTCCAA |
| | | ATGCCAGTGGAGCTAGTTCTTTAACTATAGGAGATGTAAAT |
| | | ATACCAATATCTGAACAAGATACTACAAGTACTATTGTAA |
| | | GTAAGATAAACTCCCTTTGCGCAGATAATGATATAAAGGC |
| | | TTCTTATAGTGAGATGACAGGTGAATTGATTATTTCGAGAA |
| | | AACAAACTGGTTCGTCATCAGACATTAATTTAAAAGTAATT |
| | | GGAAATGACAATTTAGCTCAGCAAATTGCTAATGATAATG |
| | | GTATCACATTTGCAAATGATGCTAGTGGAAACAAAGTGGC |
| | | AAGTGTATATGGAAAAAATCTAGAAGCTGATGTAACTGAT |
| | | GAACATGGAAGAGTAACTCATATAAGTAAAGAACAAAATT |
| | | CATTTAATATAGATAATATTGACTATAATGTAAATTCAAAA |
| | | GGAACTGCAAAGTTGACTTCTGTCACTGATACTGAAGAAG |
| | | CTGTTAAAAATATGCAAGCATTTGTGGATGATTATAATAAA |
| | | CTGATGGACAAGGTCTATGGTTTAGTTACTACTAAAAAACC |
| | | AAAAGATTATCCGCCTCTTACAGATGCCCAAAAAGAAGAT |
| | | ATGACAACTGAAGAAATAGAAAAATGGGAAA |
| 26 | Bc1A3 with | ggatccGGCGCgccgccaccATGGCATGCCCTGGCTTCCTGTGGGC |
| | 5′ | ACTTGTGATCTCCACCTGTCTTGAATTTTCCATGGCTatgagtag |
| | restriction | aaataaatattttggaccatttgatgataatgattacaacaatggctatgataaatatgatgattgtaat |
| | sites, | aatggtcgtgatgattataatagctgtgattgccatcattgctgtccaccatcatgtgtaggtccaaca |
| | Kozak, | ggcccaatgggtccaagaggtagaaccggcccaacaggaccaacgggtccaacaggtccagg |
| | HAVT20 | agtagggggaacaggcccaacaggaccaaccggtccgactggcccaacaggaaatacaggg |
| | leader, His- | aatacaggagcaacaggattaagaggtccaacaggagcaacagggggaacaggcccaacag |
| | tag | gagcgacaggagctatagggtttggagtaacaggcccaacaggcccaacaggcccaacagga |
| | sequences, | gcgacaggagcaacaggagcagatggagtaacaggtccaacaggtccaacgggagcaacag |
| | 2X stop, | gagcagatggaataacaggtccaacaggagcaacaggggcaacaggatttggagtaacaggtc |
| | and 3′ | caacaggcccaacaggagcaacaggagtaggagtaacaggagcaacaggattaataggtcca |
| | restriction | acaggagcgacaggaacacctggagcaacaggtccaacaggggcaataggagcaacaggaa |
| | sites | taggaataacaggtccaacaggagcaacaggagcaacaggggcagatggagcaacaggagta |
| | | acaggcccaacaggcccaacaggggcaacaggagcagatggagtaacaggcccaacaggag |
| | | caacaggagcaacaggaataggaataacaggcccaacaggggcaacaggagcaacaggaat |
| | | aggaataacaggagcaacagggttaataggtccaaccggagcaaccggagcaaccggagcaa |
| | | caggcccaacaggagtaacaggggcaacaggagcagcaggactaataggaccaaccggggc |
| | | aacaggagtaaccggagcagatggagcaacaggagcgacaggggcaaccggagcaacaggt |
| | | ccaacaggagcagatggattagtaggtccaacaggagcaacaggggcaacaggagcagatgg |
| | | attagtaggcccaacaggtccaacaggggcaaccggagtaggaataactggagcaaccggagc |
| | | aacaggagcgacaggtccaacaggagcagatggattagtaggtccaaccggagcgacgggag |
| | | caacaggagcagatggagtagcaggtccaaccggagcaacaggggcaacaggaaatacagg |
| | | agcagatggagcaacaggtccaacaggggcaacaggtccaacaggagcagacggattagtag |
| | | gtccaacaggagcaaccggagcaacaggattagcaggagcaaccggagcaacaggcccaata |
| | | ggagcaacaggtccaacaggagcagatggagcaacaggggcaaccggagcaacaggtccaa |
| | | caggggcagatggattagtaggtccaaccggagcaacgggagcaacaggggcaacaggtcca |
| | | acaggcccaCATCACCATCACCATCACtgatagGTTAACgctagc |
| 27 | Alr with 5′ | ggatccGGCGCGCCgccaccATGGCATGCCCTGGCTTCCTGTGGG |
| | restriction | CACTTGTGATCTCCACCTGTCTTGAATTTTCCATGGCTatgcaa |
| | sites, | aaaataacagtgcctacatgggcagagataaatctagataacttaagatttaacttaaataatattaa |
| | Kozak, | aaatttattagaagaagatattaagatttgtggagtaataaaagctgatgcatatggacatggtgcag |
| | HAVT20 | tagaagttgcaaaattgctagaaaaagaaaaagtagattacttagcagtagcaagaactgctgaag |
| | leader, His- | gaattgaacttagacaaaatggcataacacttectattttgaacttgggatatactccagacgaagct |
| | tag | tttgaagattctataaaaaataaaataactatgacagtttattctttagaaacagcacaaaagataaat |
| | sequences, | gaaattgcaaaatctttaggagaaaaagcctgtgttcatgttaaaatagactcagggatgactagaa |
| | 2X stop, | taggtttccaacctaatgaggagtcagtacaggaaataatagaattaaataaattagaatatatcgat |
| | and 3′ | ttagaaggtatgtttactcattttgctacagctgatgaagtaagtaaagagtacacttataaacaagct |
| | restriction | aataattataaatttatgtctgataaattagatgaggctggtgtaaaaatagctataaaacatgtatcaa |
| | sites | acagtgcagctattatggattgccctgatttaagattaaatatggtaagagcaggaataatattatatg |
| | | gtcattatccatctgatgatgtatttaaagatagattagaattaagaccagccatgaaattaaaatcaa |
| | | aaatcggacatataaaacaagttgaaccaggtgtaggaataagttatggactaaaatacacaacta |
| | | caggtaaagaaacaatagctacagttccaataggatacgcagatggatttactagaatccaaaaaa |
| | | atccaaaggttcttattaagggagaagtgtttgatgtagttggtagaatatgtatggatcaaataatgg |
| | | ttagaattgacaaagatatagacataaaagttggagatgaggttatactatttggagaaggcgaagt |
| | | tacagctgagcgtatagctaaagacttaggaactataaactatgaagtgttatgtatgatatcaagaa |
| | | gagttgaccgtgtttatatggaaaataatgagcttgtacaaataaacagttatttgctaaaaCATC |
| | | ACCATCACCATCACtgatagGTTAACgctagc |
| 28 | S1pA | ggatccGGCGCgccgccaccATGGCATGCCCTGGCTTCCTGTGGGC |
| | Paralogue | ACTTGTGATCTCCACCTGTCTTGAATTTTCCATGGCTgcagaaa |
| | with 5′ | ctacacaggtaaaaaaagaaacaataactaagaaagaagctacagaactagtttcgaaagttaga |
| | restriction | gatttaatgtctcaaaagtatactggtggttctcaagttggacaaccaatatatgaaataaaagttggc |
| | sites, | gagactttatcaaaattaaaaataataactaatatagatgaattagagaaattagtaaatgctttggga |
| | Kozak, | gaaaataaagaacttattgtaactataacagataaagggcatataacaaatagtgcaaatgaagtag |
| | HAVT20 | ttgcagaagcaactgaaaaatatgaaaattcagcagacctttccgctgaggctaattctataacaga |
| | leader, His- | aaaagctaaaactgaaactaatggaatttataaagttgcagatgtaaaagcttcatatgatagtgcta |
| | tag | aagataagttagttataactttaagagataaaacagacacagtaacttctaaaactatagagataggt |
| | sequences, | attggtgatgaaaaaattgatttaacagcaaatccagttgattcaacgggaacaaacttagaccettc |
| | 2X stop, | tacagaaggatttagagtaaataaaatcgttaaactaggtgtagcaggagctaaaaatattgatgat |
| | and 3′ | gtccaattagctgaaataactataaaaaatagtgacctaaatacagtttcaccacaagatttatatgat |
| | restriction | ggatatagattaactgttaaaggtaatatggtagcaaatggaacatcaaagtcaattagtgatatttca |
| | sites | tcaaaagattcagaaacaggaaagtataaatttactattaagtatactgatgcatctggaaaagcaat |
| | | agagcttactgtagaaagtactaatgaaaaagatttaaaagatgccaaagctgcattagaaggtaat |
| | | tcaaaggttaaattgatagctggagatgatagatatgcaactgcagtggctatagcaaaacaaaca |
| | | aaatatactgacaatatagttatagttaattcaaataaactagttgatggattagcagctacaccactt |
| | | gctcaatctaaaaaagcacctatattattagcatccgataatgaaataccaaaagtaactttagattat |
| | | ataaaagatataattaagaaaagcccatcagctaaaatatatatagtaggtggagaatcagcagtat |
| | | caaatacagctaaaaagcaattagaatcagtaactaagaatgttgaaagactagctggagatgata |
| | | gacatatgacttctgtagcagtagcaaaagctatggggtettttaaagatgcatttgtagtaggtgcg |
| | | aaaggggaggctgatgctatgagtatagctgccaaagctgctgaacttaaggctcctataatagta |
| | | aatggctggaatgatctttcagcagacgctatcaaattgatggatggaaaagagattggtatagttg |
| | | gtggttctaacaatgtatctagtcaaattgaaaatcaacttgctgatgttgataaagatagaaaagttc |
| | | aaagagttgaaggagaaacaagacacgatactaatgctaaggttatagaaacatattatggaaaat |
| | | tagataaactatatatagcaaaagatggatatggaaataatggtatgctagtagatgcattggcagc |
| | | aggacctctagcagcaggtaaaggtccaatacttctagctaaagctgatataacagactcacaaag |
| | | gaatgcacttagtaaaaaattaaatcttggtgcagaagtaactcaaataggtaatggagttgaattga |
| | | cagtaatacaaaagatagctaaaatactaggttggCATCACCATCACCATCACtg |
| | | atagGTTAACgctagc |
| 29 | CD1021 | ggatccGGCGCgccgccaccATGGCATGCCCTGGCTTCCTGTGGGC |
| | with 5′ | ACTTGTGATCTCCACCTGTCTTGAATTTTCCATGGCTacatctag |
| | restriction | caacaaaagtgttgatttatatagtgatgtatatattgaaaaatattttaacagagacaaggttatgga |
| | sites, | agttaatatagagatagatgaaagtgacttgaaggatatgaatgaaaatgctataaaagaagaattt |
| | Kozak, | aaggttgcaaaagtaactgtagatggagatacatatggaaacgtaggtataagaactaaaggaaat |
| | HAVT20 | tcaagtatatatctgtagcaaatagtgatagtgatagatacagctataagattaattttgataagtata |
| | leader, His- | atactagtcaaagtatggaagggettactcaattaaatcttaataactgttactctgacccatcttatat |
| | tag | gagagagtttttaacatatagtatttgcgaggaaatgggattagcgactccagaatttgcatatgcta |
| | sequences, | aagtctctataaatggcgaatatcatggtttgtatttggcagtagaaggattaaaagagtcttatcttg |
| | 2X stop, | aaaataattttggtaatgtaactggagacttatataagtcagatgaaggaagctcgttgcaatataaa |
| | and 3′ | ggagatgacccagaaagttactcaaacttaatcgttgaaagtgataaaaagacagctgattggtcta |
| | restriction | aaattacaaaactattaaaatctttggatacaggtgaagatattgaaaaatatcttgatgtagattctgt |
| | sites | ccttaaaaatatagcaataaatacagetttattaaaccttgatagctatcaagggagttttgcccataa |
| | | ctattatttatatgagcaagatggagtattttctatgttaccatgggattttaatatgtcatttggtggattt |
| | | agtggttttggtggaggtagtcaatctatagcaattgatgaacctacgacaggtaatttagaagatag |
| | | acctctcatatcctcgttattaaaaaatgagacatacaaaacaaaataccataaatatctggaagaga |
| | | tagtaacaaaatacctagattcagactatttagagaatatgacaacaaaattgcatgacatgatagca |
| | | tcatatgtaaaagaagacccaacagcattttatacttatgaagaatttgaaaaaaatataacatcttca |
| | | attgaagattctagtgataataagggatttggtaataaagggtttgacaacaataactctaataacagt |
| | | gattctaataataattctaatagtgaaaataagcgctctggaaatcaaagtgatgaaaaagaagttaa |
| | | tgctgaattaacatcaagcgtagtcaaagctaatacagataatgaaactaaaaataaaactacaaat |
| | | gatagtgaaagtaagaataatacagataaagataaaagtggaaatgataataatcaaaagctagaa |
| | | ggtectatgggtaaaggaggtaagtcaataccaggggttttggaagttgcagaagatatgagtaaa |
| | | actataaaatctcaattaagtggagaaacttcttcgacaaagcaaaactctggtgatgaaagttcaa |
| | | gtggaattaaaggtagtgaaaagtttgatgaggatatgagtggtatgccagaaccacctgaggga |
| | | atggatggtaaaatgccaccaggaatgggtaatatggataagggagatatgaatggtaaaaatgg |
| | | caatatgaatatggatagaaatcaagataatccaagagaagctggaggttttggcaatagaggag |
| | | gaggctctgtgagtaaaacaacaacatacttcaaaCATCACCATCACCATCACtg |
| | | atagGTTAACgctagc |
| 30 | F1iD with 5′ | ggatccGGCGCGCCgccaccATGGCATGCCCTGGCTTCCTGTGGG |
| | restriction | CACTTGTGATCTCCACCTGTGTCTTGATTTTCCATGGCTattag |
| | sites, | agacaaggaaaaagttgataaagcaaaacaagaacaacaaatcgttaaatggaagcaagaaata |
| | BamHI and | tatagaaatgttatacaagaatcaaaagatattatgataaatatctaagcgtaaattctcctaatagtat |
| | AcsI, | agtaagtgaaaaagcatactcactacaagaataaccaguctgatgaaagtattatagtagcaaaag |
| | Kozak, | gctcagctggtgcagaaaaaataaattatcaatttgcagtttctcaaatggctgaaccagcaaaattt |
| | HAVT20 | actattaaattaaattcaagtgaacctattgttcgacagttccctccaaatgccagtggagctagttctt |
| | leader, His- | taactataggagatgtaaatataccaatatctgaacaagatactacaagtactattgtaagtaagata |
| | tag | aactccctttgcgcagataatgatataaaggcttcttatagtgagatgacaggtgaattgattatttcg |
| | sequences, | agaaaacaaactggttcgtcatcagacattaatttaaaagtaattggaaatgacaatttagctcagca |
| | 2X Stop, | aattgctaatgataatggtatcacatttgcaaatgatgctagtggaaacaaagtggcaagtgtatatg |
| | and 3′ | gaaaaaatctagaagctgatgtaactgatgaacatggaagagtaactcatataagtaaagaacaaa |
| | restriction | attcatttaatatagataatattgactataatgtaaattcaaaaggaactgcaaagttgacttctgtcact |
| | sites, HpaI | gatactgaagaagctgttaaaaatatgcaagcatttgtggatgattataataaactgatggacaaggt |
| | and NheI | ctatggtttagttactactaaaaaaccaaaagattatccgcctcttacagatgcccaaaaagaagata |
| | | tgacaactgaagaaatagaaaaatgggaaaagaaagctaaagaaggtatacttagaaatgatgat |
| | | gagttaagaggttttgttgaagatattcagtctgcattttttggagatggaaaaaatattattgcattaa |
| | | gaaaactaggtatcaatgaaagcgaaaattacaataaaaaaggtcaaatatcatttaatgcagatac |
| | | tttttcaaaggctcttatagatgatagtgataaggtatacaaaacactagcaggttattcttcgaattat |
| | | gatgataagggaatgtttgaaaagctaaaagatattgtatatgaatattctggaagttcaacttctaaa |
| | | cttcctaaaaaagcaggtatagaaaaaactgcttctgctagtgaaaatgtatattcaaaacaaattgc |
| | | agagcaagaaagaaatataagcaggttagttgaaaaaatgaatgataaagagaaaaactuatgct |
| | | aaatattcagccttagaatctagttgaatcagtattctteccaaatgaattatttctcacaagcacagg |
| | | gtaatCATCACCATCACCATCACtgatagGTTAACgctagc |

### Host Immunization and Antibody Production

In some embodiments, once the Clostridium difficile spore antigen is overexpressed and purified, it is prepared as an immunogen for delivery to a host for eliciting an immune response. The host can be any animal known in the art that is useful in biotechnological screening assays and is capable of producing recoverable antibodies when administered an immunogen, such as but not limited to, rabbits, mice, rats, hamsters, goats, horses, monkeys, baboons, and humans. In one aspect, the host is transgenic and produces human antibodies, e.g., a mouse expressing the human antibody repertoire, thereby greatly facilitating the development of a human therapeutic.

As used herein, the term “antibody” refers to any immunoglobulin or intact molecule as well as to fragments thereof that bind to a specific epitope. Such antibodies include, but are not limited to polyclonal, monoclonal, chimeric, humanized, single chain, Fab, Fab′, F(ab)′ fragments and/or F(v) portions of the whole antibody and variants thereof. All isotypes are emcompassed by this term, including IgA, IgD, IgE, IgG, and IgM.

As used herein, the term “antibody fragment” refers specifically to an incomplete or isolated portion of the full sequence of the antibody which retains the antigen binding function of the parent antibody. Examples of antibody fragments include Fab, Fab′, F(ab′)2, and Fv fragments; diabodies; linear antibodies; single-chain antibody molecules; and multispecific antibodies formed from antibody fragments.

An intact “antibody” comprises at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term antibody includes antigen-binding portions of an intact antibody that retain capacity to bind. Examples of binding include (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment (Ward et al., Nature, 341:544-546 (1989)), which consists of a VH domain; and (vi) an isolated complementarity determining region (CDR).

As used herein, the term “single chain antibodies” or “single chain Fv (scFv)” refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., Science, 242:423-426 (1988); and Huston et al., Proc Natl Acad Sci USA, 85:5879-5883 (1988)). Such single chain antibodies are included by reference to the term “antibody” fragments can be prepared by recombinant techniques or enzymatic or chemical cleavage of intact antibodies.

As used herein, the term “human sequence antibody” includes antibodies having variable and constant regions (if present) derived from human germline immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT App. Pub. Nos. WO 01/14424 and WO 00/37504. However, the term “human sequence antibody”, as used herein, is not intended to include antibodies in which CDR sequences derived from the germline of another mammalian species, such as a mouse, have been grafted onto human framework sequences (e.g., humanized antibodies).

Also, recombinant immunoglobulins can be produced. See, Cabilly, U.S. Pat. No. 4,816,567, incorporated herein by reference in its entirety and for all purposes; and Queen et al., Proc Natl Acad Sci USA, 86:10029-10033 (1989).

As used herein, the term “monoclonal antibody” refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one aspect, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

As used herein, the term “antigen” refers to a substance that prompts the generation of antibodies and can cause an immune response. It can be used interchangeably in the present disclosure with the term “immunogen”. In the strict sense, immunogens are those substances that elicit a response from the immune system, whereas antigens are defined as substances that bind to specific antibodies. An antigen or fragment thereof can be a molecule (i.e., an epitope) that makes contact with a particular antibody. When a protein or a fragment of a protein is used to immunize a host animal, numerous regions of the protein can induce the production of antibodies (i.e., elicit the immune response), which bind specifically to the antigen (given regions or three-dimensional structures on the protein). The antigen can include, but is not limited to, Clostridium difficile spore proteins and fragments thereof.

As used herein, the term “humanized antibody,” refers to at least one antibody molecule in which the amino acid sequence in the non-antigen binding regions and/or the antigen-binding regions has been altered so that the antibody more closely resembles a human antibody, and still retains its original binding ability.

In addition, techniques developed for the production of “chimeric antibodies” (Morrison, et al., Proc Natl Acad Sci, 81:6851-6855 (1984), incorporated herein by reference in their entirety) by splicing the genes from a mouse antibody molecule of appropriate antigen specificity together with genes from a human antibody molecule of appropriate biological activity can be used. For example, the genes from a mouse antibody molecule specific for an autoinducer can be spliced together with genes from a human antibody molecule of appropriate biological activity. A chimeric antibody is a molecule in which different portions are derived from different animal species, such as those having a variable region derived from a murine mAb and a human immunoglobulin constant region.

In addition, techniques have been developed for the production of humanized antibodies (see, e.g., U.S. Pat. No. 5,585,089 and U.S. Pat. No. 5,225,539, which are incorporated herein by reference in their entirety). An immunoglobulin light or heavy chain variable region consists of a “framework” region interrupted by three hypervariable regions, referred to as complementarity determining regions (CDRs). Briefly, humanized antibodies are antibody molecules from non-human species having one or more CDRs from the non-human species and a framework region from a human immunoglobulin molecule.

Alternatively, techniques described for the production of single chain antibodies can be adapted to produce single chain antibodies against an immunogenic conjugate of the present disclosure. Single chain antibodies are formed by linking the heavy and light chain fragments of the Fv region via an amino acid bridge, resulting in a single chain polypeptide. Fab and F(ab′)2 portions of antibody molecules can be prepared by the proteolytic reaction of papain and pepsin, respectively, on substantially intact antibody molecules by methods that are well-known. See e.g., U.S. Pat. No. 4,342,566. Fab′ antibody molecule portions are also well-known and are produced from F(ab′)2 portions followed by reduction of the disulfide bonds linking the two heavy chain portions as with mercaptoethanol, and followed by alkylation of the resulting protein mercaptan with a reagent such as iodoacetamide.

### Antibody Assays

After the host is immunized and allowed to elicit an immune response to the immunogen, a screening assay can be performed to determine if the desired antibodies are being produced. Such assays may include assaying the antibodies of interest to confirm their specificity and affinity and to determine whether those antibodies cross-react with other proteins.

The terms “specific binding” or “specifically binding” refer to the interaction between the antigen and their corresponding antibodies. The interaction is dependent upon the presence of a particular structure of the protein recognized by the binding molecule (i.e., the antigen or epitope). In order for binding to be specific, it should involve antibody binding of the epitope(s) of interest and not background antigens.

Once the antibodies are produced, they are assayed to confirm that they are specific for the antigen of interest and to determine whether they exhibit any cross reactivity with other antigens. One method of conducting such assays is a sera screen assay as described in U.S. App. Pub. No. 2004/0126829, the contents of which are hereby expressly incorporated herein by reference. However, other methods of assaying for quality control are within the skill of a person of ordinary skill in the art and therefore are also within the scope of the present disclosure.

Antibodies, or antigen-binding fragments, variants or derivatives thereof of the present disclosure can also be described or specified in terms of their binding affinity to an antigen. The affinity of an antibody for an antigen can be determined experimentally using any suitable method. (See, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer.

The affinity binding constant (K_{aff}) can be determined using the following formula:

$${K}_{aff} = \frac{( n - 1 )}{2 ( {n [ {mAb}^{′} ]}_{t} - {[ mAb ]}_{t} )}$$

in which:

$$n = \frac{{[ mAg ]}_{t}}{{[ {mAg}^{′} ]}_{t}}$$

[mAb] is the concentration of free antigen sites, and [mAg] is the concentration of free monoclonal binding sites as determined at two different antigen concentrations (i.e., [mAg]ₜ and [mAg′]ₜ) (Beatty et al., J Imm Meth, 100:173-179 (1987)).

The term “high affinity” for an antibody refers to an equilibrium association constant (K_{aff}) of at least about 1×10⁷ liters/mole, or at least about 1×10⁸ liters/mole, or at least about 1×10⁹ liters/mole, or at least about 1×10¹⁰ liters/mole, or at least about 1×10¹¹ liters/mole, or at least about 1×10¹² liters/mole, or at least about 1×10¹³ liters/mole, or at least about 1×10¹⁴ liters/mole or greater. “High affinity” binding can vary for antibody isotypes. K_{D}, the equilibrium dissociation constant, is a term that is also used to describe antibody affinity and is the inverse of K_{aff}.

### Adjuvants

Compositions of the present invention can include adjuvants to further increase the immunogenicity of one or more of the Clostridium difficile spore antigen proteins. Such adjuvants include any compound or compounds that act to increase an immune response to peptides or combination of peptides, thus reducing the quantity of antigen necessary in the composition, and/or the frequency of injection necessary in order to generate an adequate immune response. Suitable adjuvants include those suitable for use in mammals, preferably in humans. Examples of known suitable adjuvants that can be used in humans include, but are not necessarily limited to, alum, aluminum phosphate, aluminum hydroxide, MF59 (4.3% w/v squalene, 0.5% w/v polysorbate 80 (Tween 80), 0.5% w/v sorbitan trioleate (Span 85)), CpG-containing nucleic acid, QS21 (saponin adjuvant), MPL (Monophosphoryl Lipid A), 3DMPL (3-O-deacylated MPL), extracts from Aquilla, ISCOMS (see, e.g., Sjolander et al. (1998) J. Leukocyte Biol. 64:713; WO90/03184, WO96/11711, WO 00/48630, WO98/36772, WO00/41720, WO06/134423 and WO07/026190), LT/CT mutants, poly(D,L-lactide-co-glycolide) (PLG) microparticles, Quil A, interleukins, and the like. For veterinary applications including but not limited to animal experimentation, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59 (WO90/14837; Chapter 10 in Vaccine design: the subunit and adjuvant approach, eds. Powell & Newman, Plenum Press 1995), containing 5% Squalene, 0.5% Tween 80 (polyoxyethylene sorbitan mono-oleate), and 0.5% Span 85 (sorbitan trioleate) (optionally containing muramyl tri-peptide covalently linked to dipalmitoyl phosphatidylethanolamine (MTP-PE)) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX); (2) saponin adjuvants, such as QS21, STIMULON (Cambridge Bioscience, Worcester, Mass.), Abisco (Isconova, Sweden), or Iscomatrix (Commonwealth Serum Laboratories, Australia), may be used or particles generated therefrom such as ISCOMs (immunostimulating complexes), which ISCOMS may be devoid of additional detergent e.g. WO00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO00/56358; (6) combinations of 3dMPL with, for example, QS21 and/or oil-in-water emulsions e.g. EP-A-0835318, EP-A-0735898, EP-A-0761231; (7) oligonucleotides comprising CpG motifs [Krieg Vaccine 2000, 19, 618-622; Krieg Curr opin Mol Ther 2001 3:15-24; Roman et al., Nat. Med., 1997, 3, 849-854; Weiner et al., PNAS USA, 1997, 94, 10833-10837; Davis et al, J. Immunol, 1998, 160, 870-876; Chu et al., J. Exp. Med, 1997, 186, 1623-1631; Lipford et al, Ear. J. Immunol., 1997, 27, 2340-2344; Moldoveami e/al., Vaccine, 1988, 16, 1216-1224, Krieg et al., Nature, 1995, 374, 546-549; Klinman et al., PNAS USA, 1996, 93, 2879-2883; Ballas et al, J. Immunol, 1996, 157, 1840-1845; Cowdery et al, J. Immunol, 1996, 156, 4570-4575; Halpern et al, Cell Immunol, 1996, 167, 72-78; Yamamoto et al, Jpn. J. Cancer Res., 1988, 79, 866-873; Stacey et al, J. Immunol., 1996, 157, 2116-2122; Messina et al, J. Immunol, 1991, 147, 1759-1764; Yi et al, J. Immunol, 1996, 157, 4918-4925; Yi et al, J. Immunol, 1996, 157, 5394-5402; Yi et al, J. Immunol, 1998, 160, 4755-4761; and Yi et al, J. Immunol, 1998, 160, 5898-5906; International patent applications WO96/02555, WO98/16247, WO98/18810, WO98/40100, WO98/55495, WO98/37919 and WO98/52581] i.e. containing at least one CG dinucleotide, where the cytosine is unmethylated; (8) a polyoxyethylene ether or a polyoxyethylene ester e.g. WO99/52549; (9) a polyoxyethylene sorbitan ester surfactant in combination with an octoxynol (WO01/21207) or a polyoxyethylene alkyl ether or ester surfactant in combination with at least one additional non-ionic surfactant such as an octoxynol (WO01/21152); (10) a saponin and an immunostimulatory oligonucleotide (e.g. a CpG oligonucleotide) (WO00/62800); (11) an immunostimulant and a particle of metal salt e.g. WO00/23105; (12) a saponin and an oil-in-water emulsion e.g. WO99/11241; (13) a saponin (e.g. QS21)+3dMPL+IM2 (optionally+a sterol) e.g. WO98/57659; (14) other substances that act as immunostimulating agents to enhance the efficacy of the composition, such as Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), (15) ligands for toll-like receptors (TLR), natural or synthesized (e.g. as described in Kanzler et al 2007, Nature Medicine 13, p 1552-9), including TLR3 ligands such as polyl:C and similar compounds such as Hiltonol and Ampligen.

Adjuvants can also include for example, emulsifiers, muramyl dipeptides, avridine, aqueous adjuvants such as aluminum hydroxide, chitosan-based adjuvants, and any of the various saponins, oils, and other substances known in the art, such as Amphigen, LPS, bacterial cell wall extracts, bacterial DNA, synthetic oligonucleotides and combinations thereof (Schijns et al., Curr. Opi. Immunol. (2000) 12: 456), Mycobacterialphlei (M. phlei) cell wall extract (MCWE) (U.S. Pat. No. 4,744,984), M. phlei DNA (M-DNA), M-DNA-M. phlei cell wall complex (MCC). For example, compounds which can serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrhethylammonlum bromide, while synthetic nonionic agents are exemplified by glycerylesters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil can be a mineral oil, a vegetable oil, or an animal oil. Mineral oil, or oil-in-water emulsions in which the oil component is mineral oil are preferred. In this regard, a “mineral oil” is defined herein as a mixture of liquid hydrocarbons obtained from petrolatum via a distillation technique; the term is synonymous with “liquid paraffin,” “liquid petrolatum” and “white mineral oil.” The term is also intended to include “light mineral oil,” i.e., an oil which is similarly obtained by distillation of petrolatum, but which has a slightly lower specific gravity than white mineral oil. See, e.g., Remington's Pharmaceutical Sciences, supra. A particularly preferred oil component is the oil-in-water emulsion sold under the trade name of EMULSIGEN PLUS™ (comprising a light mineral oil as well as 0.05% formalin, and 30 mcg/mL gentamicin as preservatives), available from MVP Laboratories, Ralston, Nebr. Suitable animal oils include, for example, cod liver oil, halibut oil, menhaden oil, orange roughy oil and shark liver oil, all of which are available commercially. Suitable vegetable oils, include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include mines, quaternary ammonium compounds, guanidines, benzamidines and thiouroniums (Gall, D. (1966) Immunology 11: 369-386). Specific compounds include dimethyldioctadecylammoniumbromide (DDA) (available from Kodak) and N,N-dioctadecyl-N,N-bis(2-hydroxyethyl)propanediine (“avridine”). The use of DDA as an immunologic adjuvant has been described; see, e.g., the Kodak Laboratory Chemicals Bulletin 56(1): 1-5 (1986); Adv. Drug Deliv. Rev. 5(3):163-187 (1990); J. Controlled Release 7: 123-132 (1988); Clin. Exp. Immunol. 78(2): 256-262 (1989); J. Immunol. Methods 97(2): 159-164 (1987); Immunology 58(2): 245-250 (1986); and Int. Arch. Allergy Appl. Immunol. 68(3): 201-208 (1982). Avridine is also a well-known adjuvant. See, e.g., U.S. Pat. No. 4,310,550 to Wolff, III et al., which describes the use of N,N-higher alkyl-N′,N′-bis(2-hydroxyethyl)propane diamines in general, and avridine in particular, as vaccine adjuvants. U.S. Pat. No. 5,151,267 to Babiuk, and Babiuk et al. (1986) Virology 159: 57-66, also relate to the use of avridine as a vaccine adjuvant.

An adjuvant for use with the vaccine is “VSA3” which is a modified form of the EMULSIGEN PLUS™ adjuvant which includes DDA (see, U.S. Pat. No. 5,951,988, incorporated herein by reference in its entirety).

Compositions including one or more of peptides in aspects of the present invention can be prepared by uniformly and intimately bringing into association the composition preparations and the adjuvant using techniques well known to those skilled in the art including, but not limited to, mixing, sonication and microfluidation. The adjuvant will preferably comprise about 10 to 50% (v/v) of the composition, more preferably about 20 to 40% (v/v) and most preferably about 20 to 30% or 35% (v/v), or any integer within these ranges.

### Pharmaceutical Compositions

An aspect of the invention provides a composition comprising an effective immunizing amount of an isolated Clostridium difficile spore antigen protein, or an isolated nucleic acid encoding such antigenic proteins, and a pharmaceutically acceptable carrier, wherein the composition is effective in a vertebrate subject to reduce, eliminate, or prevent Clostridium difficile bacterial infection. A further aspect provides pharmaceutical compositions comprising antibodies directed against Clostridium difficile spore antigen proteins for providing passive immunity to Clostridium difficile infection.

The compositions of the present invention are normally prepared as injectables, either as liquid solutions or suspensions, or as solid forms which are suitable for solution or suspension in liquid vehicles prior to injection. The preparation can also be prepared in solid form, emulsified or the active ingredient encapsulated in liposome vehicles or other particulate carriers used for sustained delivery. For example, the vaccine can be in the form of an oil emulsion, water in oil emulsion, water-in-oil-in-water emulsion, site-specific emulsion, long-residence emulsion, stickyemulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of the vaccine.

Polypeptides are formulated into compositions for delivery to a mammalian subject. The composition is administered alone, and/or mixed with a pharmaceutically acceptable vehicle or excipient. Suitable vehicles are, for example, water, saline, dextrose, glycerol, ethanol, or the like, and combinations thereof. In addition, the vehicle can contain minor amounts of auxiliary substances such as wetting or emulsifying agents, pH buffering agents, or adjuvants in the case of compositions, which enhance the effectiveness of the composition. Suitable adjuvants are described above. The compositions of the present invention can also include ancillary substances, such as pharmacological agents, cytokines, or other biological response modifiers.

Furthermore, the compositions including, for example, one or more Clostridium difficile spore antigens can be formulated into compositions in either neutral or salt forms. Pharmaceutically acceptable salts include the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Actual methods of preparing such dosage forms are known, or will be apparent, to those skilled in the art. See, e.g., Remington's Pharmaceutical Sciences, Mack Publishing Company, Easton, Pa., current edition.

The composition is formulated to contain an effective amount of a protein, the exact amount being readily determined by one skilled in the art, wherein the amount depends on the animal to be treated and the capacity of the animal's immune system to synthesize antibodies. The composition or formulation to be administered will contain a quantity of one or more secreted proteins adequate to achieve the desired state in the subject being treated. For purposes of the present invention, a therapeutically effective amount of a composition comprising a protein, contains about 0.05 to 1500 μg protein, preferably about 10 to 1000 μg protein, more preferably about 30 to 500 μg and most preferably about 40 to 300 μg, or any integer between these values. For example, peptides of the invention can be administered to a subject at a dose of about 0.1 μg to about 200 mg, e.g., from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg, with optional boosters given at, for example, 1 week, 2 weeks, 3 weeks, 4 weeks, two months, three months, 6 months and/or a year later. For prophylaxis purposes, the amount of peptide in each dose is selected as an amount which induces an immunoprotective response without significant adverse side effects in typical vaccinees. Following an initial vaccination, subjects may receive one or several booster immunisations adequately spaced. It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

Routes of administration include, but are not limited to, oral, topical, subcutaneous, intramuscular, intravenous, subcutaneous, intradermal, transdermal and subdermal. Depending on the route of administration, the volume per dose is preferably about 0.001 to 10 ml, more preferably about 0.01 to 5 ml, and most preferably about 0.1 to 3 ml. Compositions can be administered in a single dose treatment or in multiple dose treatments (boosts) on a schedule and over a time period appropriate to the age, weight and condition of the subject, the particular vaccine formulation used, and the route of administration.

In some embodiments, a single dose of polypeptide or pharmaceutical composition according to the invention is administered. In other embodiments, multiple doses of a peptide or pharmaceutical composition according to the invention are administered. The frequency of administration can vary depending on any of a variety of factors, e.g., severity of the symptoms, degree of immunoprotection desired, whether the composition is used for prophylactic or curative purposes, etc. For example, in some embodiments, a peptide or pharmaceutical composition according to the invention is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). When the composition of the invention is used for prophylaxis purposes, they will be generally administered for both priming and boosting doses. It is expected that the boosting doses will be adequately spaced, or preferably given yearly or at such times where the levels of circulating antibody fall below a desired level. Boosting doses may consist of the peptide in the absence of the original immunogenic carrier molecule. Such booster constructs may comprise an alternative immunogenic carrier or may be in the absence of any carrier. Such booster compositions may be formulated either with or without adjuvant.

The duration of administration of a polypeptide according to the invention, e.g., the period of time over which a peptide is administered, can vary, depending on any of a variety of factors, e.g., patient response, etc. For example, a polypeptide can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Any suitable pharmaceutical delivery means can be employed to deliver the compositions to the vertebrate subject. For example, conventional needle syringes, spring or compressed gas (air) injectors (U.S. Pat. No. 1,605,763 to Smoot; U.S. Pat. No. 3,788,315 to Laurens; U.S. Pat. No. 3,853,125 to Clark et al.; U.S. Pat. No. 4,596,556 to Morrow et al.; and U.S. Pat. No. 5,062,830 to Dunlap), liquid jet injectors (U.S. Pat. No. 2,754,818 to Scherer; U.S. Pat. No. 3,330,276 to Gordon; and U.S. Pat. No. 4,518,385 to Lindcaner et al.), and particle injectors (U.S. Pat. No. 5,149,655 to McCabe et al. and U.S. Pat. No. 5,204,253 to Sanford et al.) are all appropriate for delivery of the compositions.

If a jet injector is used, a single jet of the liquid vaccine composition is ejected under high pressure and velocity, e.g., 1200-1400 PSI, thereby creating an opening in the skin and penetrating to depths suitable for immunization.

The compositions, or nucleic acids, or polypeptides, or antibodies can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of the composition or nucleic acids, peptides, polypeptides, or antibodies are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Compositions or nucleic acids, polypeptides, or antibodies, when administered orally, can be protected from digestion. This can be accomplished either by complexing the nucleic acid, polypeptide, or antibody with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani, Crit. Rev. Ther. Drug Carrier Syst. 13: 85-184, 1996. For topical, transdermal administration, the agents are formulated into ointments, creams, salves, powders and gels. Transdermal delivery systems can also include, e.g., patches.

Compositions or nucleic acids, polypeptides, or antibodies as aspects of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention (see, e.g., Putney, Nat. Biotechnol. 16: 153-157, 1998).

For inhalation, compositions or nucleic acids, nucleic acids, polypeptides, or antibodies as aspects of the invention can be delivered using any system known in the art, including dry powder aerosols, liquids delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

Compositions or nucleic acids, polypeptides, or antibodies of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun, Anesth Analg. 85: 317-323, 1997). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's, See also, Bai, J. Neuroimmunol. 80: 65-75, 1997; Warren, J. Neurol. Sci. 152: 31-38, 1997; Tonegawa, J. Exp. Med. 186: 507-515, 1997.

In one aspect, the pharmaceutical formulations comprising compositions or nucleic acids, polypeptides, or antibodies of the invention are incorporated in lipid monolayers or bilayers, e.g., liposomes, see, e.g., U.S. Pat. Nos. 6,110,490; 6,096,716; 5,283,185; 5,279,833. Aspects of the invention also provide formulations in which water soluble nucleic acids, peptides or polypeptides of the invention have been attached to the surface of the monolayer or bilayer. For example, peptides can be attached to hydrazide-PEG-(distearoylphosphatidyl) ethanolamine-containing liposomes (see, e.g., Zalipsky, Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the nucleic acid, peptides and/or polypeptides of the invention are incorporated within micelles and/or liposomes (see, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art, see, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4,235,871, 4,501,728 and 4,837,028.

In one aspect, the compositions are prepared with carriers that will protect the protein against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models, e.g., of inflammation or disorders involving undesirable inflammation, to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography, generally of a labeled agent. Animal models useful in studies, e.g., preclinical protocols, are known in the art, for example, animal models for inflammatory disorders such as those described in Sonderstrup (Springer, Sem. Immunopathol. 25: 35-45, 2003) and Nikula et al., Inhal. Toxicol. 4(12): 123-53, 2000).

As defined herein, a therapeutically effective amount of vaccine compositions, protein or polypeptide such as an antibody (i.e., an effective dosage) ranges from about 0.001 to 30 mg/kg body weight, for example, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The protein or polypeptide can be administered one or several times per day or per week for between about 1 to 10 weeks, for example, between 2 to 8 weeks, between about 3 to 7 weeks, or about 4, 5, or 6 weeks. In some instances the dosage can be required over several months or more. The skilled artisan will appreciate that certain factors can influence the dosage and timing required to effectively treat a subject, including, but not limited to the severity of the disease or disorder, previous treatments, the general health and/or age of the subject, and other diseases present. Moreover, treatment of a subject with a therapeutically effective amount of an agent such as a protein or polypeptide (including an antibody) can include a single treatment or, preferably, can include a series of treatments.

For antibodies, the dosage is generally about 10 mg/kg of body weight (for example, 10 mg/kg to 20 mg/kg). Partially human antibodies and fully human antibodies generally have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology, 14: 193, 1997).

Aspects of present invention encompass compositions comprising an effective immunizing amount of an isolated Clostridium difficile spore antigen protein and a pharmaceutically acceptable carrier, wherein said composition is effective in a vertebrate subject to reduce or eliminate Clostridium difficile bacterial infection.

The pharmaceutical compositions can be included in a container, pack, or dispenser together with instructions for administration.

Compounds as described herein can be used for the preparation of a medicament for use in any of the methods of treatment described herein.

The pharmaceutical compositions are generally formulated as sterile, substantially isotonic and in full compliance with all Good Manufacturing Practice (GMP) regulations of the U.S. Food and Drug Administration.

### Treatment Regimens: Pharmacokinetics

The pharmaceutical composition aspects of the invention can be administered in a variety of unit dosage forms depending upon the method of administration. Dosages for typical vaccine compositions or nucleic acids, peptide and polypeptide, and antibody pharmaceutical compositions are well known to those of skill in the art. Such dosages are typically advisory in nature and are adjusted depending on the particular therapeutic context or patient tolerance. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

In therapeutic applications, compositions are administered to a patient at risk for Clostridium difficile bacterial infection or suffering from active infection in an amount sufficient to at least partially arrest or prevent the condition or a disease and/or its complications. For example, in one aspect, a vaccine composition comprising a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

### Methods of Treatment

Also described herein are both prophylactic and therapeutic methods of treating a subject at risk of (or susceptible to) a disorder or a method of preventing or treating a Clostridium difficile bacterial infection by administering a composition of the invention.

Prophylactic Methods

An aspect of the invention relates to methods for preventing or treating in a subject a Clostridium difficile bacterial infection or bacterial carriage or both by administering a composition comprising an effective immunizing amount of protein and pharmaceutically acceptable carrier, wherein the composition is effective in a vertebrate subject to reduce or eliminate Clostridium difficile bacterial infection. Subjects at risk for a disorder or undesirable symptoms that are caused or contributed to by Clostridium difficile bacterial infection and bacterial carriage can be identified by, for example, any of a combination of diagnostic or prognostic assays as described herein or are known in the art. In general, such disorders involve gastrointestinal disorders such as bloating, diarrhea, and abdominal pain. Administration of the agent as a prophylactic agent can occur prior to the manifestation of symptoms, such that the symptoms are prevented, delayed, or diminished compared to symptoms in the absence of the agent.

Therapeutic Methods

An aspect of the invention relates to methods for preventing or treating in a subject a Clostridium difficile bacterial infection or bacterial carriage by administering a composition comprising an effective immunizing amount of a protein and a pharmaceutically acceptable carrier, wherein the composition is effective in a vertebrate subject to reduce or eliminate Clostridium difficile bacterial infection. In another embodiment relates to methods for preventing or treating in a subject a Clostridium difficile bacterial infection or bacterial carriage by administering a composition comprising an effective amount of an antibody and a pharmaceutically acceptable carrier, wherein the composition is effective in a vertebrate subject to reduce or eliminate Clostridium difficile bacterial infection.

### Kits

The invention provides kits comprising the compositions, e.g., nucleic acids, expression cassettes, vectors, cells, polypeptides, and antibodies. The kits also can contain instructional material teaching the methodologies and uses of the invention, as described herein.

The following examples of specific aspects for carrying out the present invention are offered for illustrative purposes only, and are not intended to limit the scope of the present invention in any way.

### EXAMPLES

### Example 1

### Expression and Purification of C. difficile BclA3 Protein

A C. difficile BclA3 sequence from the hypervirulent strain R20291 was obtained from the NCBI public database (accession number: FN545816 (region: 3807430-3809466)). Using standard molecular biological methods, the signal peptide and transmembrane regions of the BclA3 gene were removed and an HAVT20 leader sequence, His-tags, and Kozak sequence were added before cloning the construct into the pcDNA3002Neo plasmid using AscI and HpaI restriction enzyme sites (SEQ ID NO:23). The sequence of BclA3 was subsequently codon optimized for mammalian cell expression.

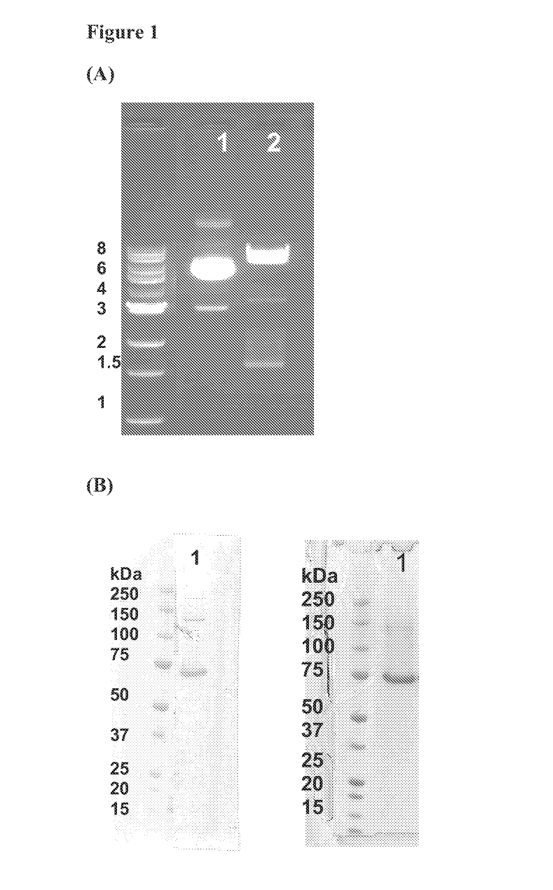

Plasmid DNA corresponding to BclA3 was extracted from a culture grown from a glycerol stock using an EndoFree Giga kit from Qiagen. The identity of the plasmid DNA was confirmed by restriction digestion with AscI and HpaI restriction enzymes (see FIG. 1A).

A large scale transfection (300 ml) was performed in HEK293F cells for large scale expression of BclA3 protein. A total of 3×10⁸ cells were transfected with 300 μg of BclA3 plasmid DNA. The supernatant was harvested by centrifugation at 3 days and 7 days post-transfection. The transfected supernatant was filtered through a 0.22 μm filter and purified on a Ni column (HisTRAP HP, GE Healthcare) using the AktaPurifier FPLC. (See Table 3 for FPLC procedure.) The eluted protein was buffer exchanged into D-PBS and protein concentration was determined by BCA assay. A total of 16 mg of protein was purified from a 300 ml culture. The purified protein was run on SDS-PAGE for size determination and also transferred to a nitrocellulose membrane, which was probed with an anti-His-tag antibody to confirm that a protein of the correct size containing a His-tag had been obtained (see FIG. 1B). We found that a protein of larger than predicted size was obtained, which is likely due to the protein having been glycosylated by expression within mammalian cells. Mass spectrometry is used for further confirmation of the identity of the protein.

TABLE 3
Procedure for HisTRAP HP Purification of transfected supernatants

| Block | Variable | Value | Range |
|---|---|---|---|
| Main | Column | HisTrap_HP_5_ml | |
| Start_with_PumpWash_Basic | Wash_Inlet_A On | | |
| | Wash_Inlet_B On | | |
| Flow_Rate | Flow_Rate {ml/min} | 5.000 | 0.000-10.000 |
| Column_Pressure_Limit | Column_PressureLimit {MPa} | 0.30 | 0.00-25.00 |
| Start_Instructions | Averaging_Time_UV | 5.10 | |
| Alarm_Sample_PressureLimit | Sample_PressureLimit {MPa} | 0.30 | 0.00-2.00 |
| Start_Conc_B | Start_ConcB {% B} | 0.0 | 0.0-100.0 |
| Column_Equilibration | Equilibrate_with {CV} | 5.00 | 0.00-999999.00 |
| Aut_PressureFlow_Regulation | System_Pump | Normal | |
| | System_PressLevel {MPa} | 0.00 | 0.00-25.00 |
| | System_MinFlow {ml/min} | 0.000 | 0.000-10.000 |
| Flowthrough_Fractionation | Flowthrough_TubeType | 30 mm | |
| | Flowthrough_FracSize {ml} | 50.000 | 0.000-99999.000 |
| | Flowthrough_StartAt | FirstTube | |
| Direct_Sample_Loading | Injection_Flowrate {ml/min} | 5.0 | 0.0-50.0 |
| | Volume_of_Sample {ml} | 100.0 | 0.0-20000.0 |
| PressureReg_ Sample_Pump | Sample_PumpPressFlowControl | | |
| | Sample_Min_Flow {ml/min} | 0.1 | 0.1-49.9 |
| Wash_Out_Unbound_Sample | Wash_column_with {CV} | 2.00 | 0.00-999999.00 |
| Wash_Basic_1 | 1_Wash_Inlet_A | OFF | |
| | 1_Wash_Inlet_B | OFF | |
| ConcB_Step_1 | 1_ConcB_Step {%B} | 0.0 | 0.0-100.0 |
| Fractionation_Segment_1 D | 1_Tube_Type 30 mm | | |
| | 1_Fraction_Size {ml} | 50.000 | 0.000-99999.000 |
| | 1_Start_at | NextTube | |
| | 1_PeakFrac_TubeType | 18 mm | |
| | 1_PeakFraction_Size {ml} | 0.000 | 0.000-99999.000 |
| | 1_PeakFrac_Start_at | NextTube | |
| Step_1 | 1_Length_of_Step {CV} | 15.00 | 0.00-999999.00 |
| Wash_Basic_2 | 2_Wash_Inlet_A | OFF | |
| | 2_Wash_Inlet_B | OFF | |
| Fractionation_Segment_2 | 2_Tube_Type | 18 mm | |
| | 2_Fraction_Size {ml} | 5.000 | 0.000-99999.000 |
| | 2_Start_at | FirstTube | |
| | 2_PeakFrac_TubeType | 18 mm | |
| | 2_PeakFraction_Size {ml} | 0.000 | 0.000-99999.000 |
| | 2_PeakFrac_Start_at | NextTube | |
| Gradient_Segment_2 | Target_ConcB_2 {% B} | 100.0 | 0.0-100.0 |
| | Length_of_Gradient_2 {CV} | 5.000 | 0.000-99999.000 |
| Wash_Basic_3 | 3_Was_Inlet_A | OFF | |
| | 3_Wash_Inlet_B | OFF | |
| ConcB_Step_3 | 3_ConcB_Step {% B} | 100.0 | 0.0-100.0 |
| Fractionation_Segment_3 D | 3 Tube Type | 18 mm | |
| | 3_Fraction_Size {ml} | 2.000 | 0.000-99999.000 |
| | 3_Start_at | NextTube | |
| | 3_PeakFrac_TubeType | 18 mm | |
| | 3_PeakFraction_Size {ml} | 0.000 | 0.000-99999.000 |
| | 3_PeakFrac_Start_at | NextTube | |
| Step_3 | 3_Length_of_Step {CV} | 5.00 | 0.00-999999.00 |
| Gradient_Delay | Gradient_Delay {ml} | 3.00 | 0.00-999999.00 |

### Example 2

### Expression and Purification of C. difficile Alr Protein

A C. difficile Alr sequence from the hypervirulent strain R20291 was obtained from the NCBI public database (accession number: FN545816 (region: 3936313-3937470)). Using standard molecular biological methods, the signal peptide and transmembrane regions of the Alr gene were removed and an HAVT20 leader sequence, His-tags, and Kozak sequence were added before cloning the construct into the pcDNA3002Neo plasmid using AscI and HpaI restriction enzyme sites (SEQ ID NO:24). The sequence of Alr was subsequently codon optimized for mammalian cell expression.

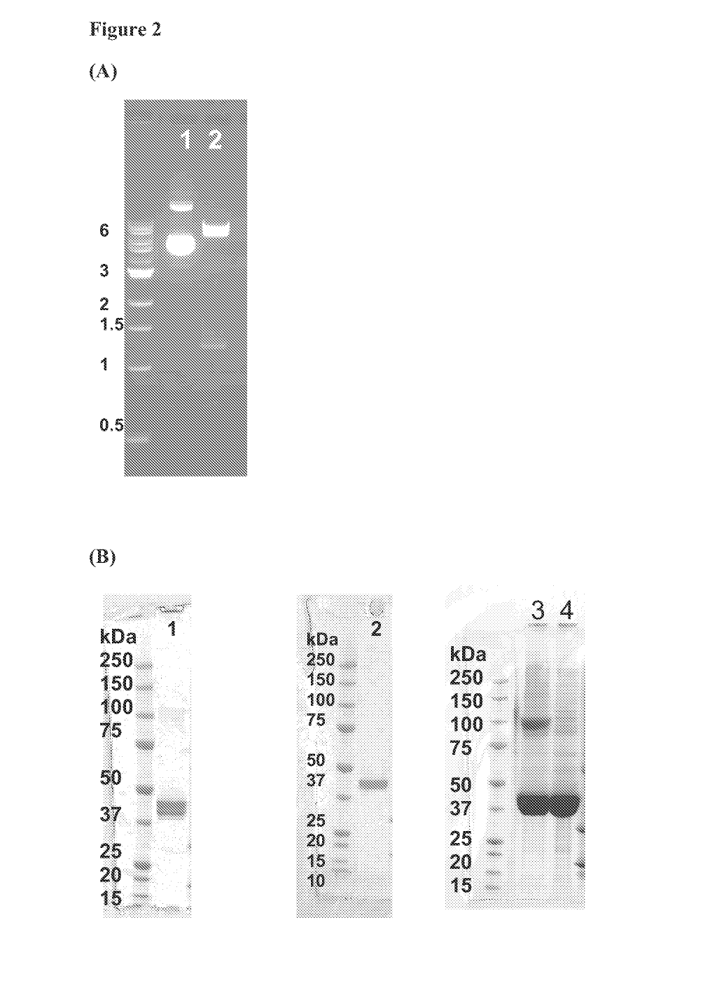

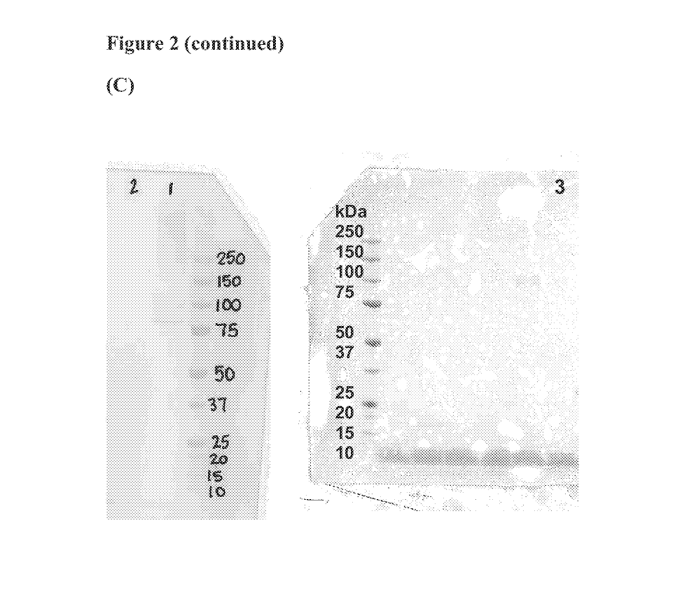

Plasmid DNA corresponding to Alr was extracted from a culture grown from a glycerol stock using an EndoFree Giga kit from Qiagen. The identity of the plasmid DNA was confirmed by restriction digestion with AscI and HpaI restriction enzymes (see FIG. 2A).

A large scale transfection (300 ml) was performed in HEK293F cells for large scale expression of Alr protein. A total of 3×10⁸ cells were transfected with 300 μg of Alr plasmid DNA. The supernatant was harvested by centrifugation (3000 rpm for 15 min at room temperature) at 3 days and 7 days post-transfection. The transfected supernatant was filtered through a 0.22 μm filter and purified on a Ni column (HisTRAP HP, GE Healthcare) using the AktaPurifier FPLC (See Table 3 for FPLC procedure). The eluted protein was buffer exchanged into D-PBS and protein concentration was determined by BCA assay. A total of 34 mg of protein was purified from a 300 ml culture. Of interest was the fact that the eluted protein was a distinct yellow color that became more intense as the protein was concentrated. The purified protein was run on SDS-PAGE for size determination and also transferred to a nitrocellulose membrane, which was probed with an anti-His-tag antibody to confirm that a protein of the correct size containing a His-tag had been obtained (see FIG. 2C). We found that while the protein ran at the correct size, it would not bind the anti-His-tag antibody, which could be due to the folding of the protein. We have evidence that protein clumping may be occurring as the larger bands observed with a non-reduced sample the gel were resolved to the correct sized band in a sample treated with beta-mercaptoethanol (FIG. 2B). Mass spectrometry is used to confirm the identity of the protein.

### Example 3

### Expression and Purification of C. difficile SlpA Paralogue Protein

A C. difficile SlpA paralogue sequence from the hypervirulent strain 820291 was obtained from the NCBI public database (accession number: FN545816 (region: 3157304-3159175)). Using standard molecular biological methods, the signal peptide and transmembrane regions of the SlpA paralogue gene were removed and an HAVT20 leader sequence, His-tags, and Kozak sequence were added before cloning the construct into the pcDNA3002Neo plasmid using AscI and HpaI restriction enzyme sites (SEQ ID NO:25). The sequence of SlpA paralogue was subsequently codon optimized for mammalian cell expression.

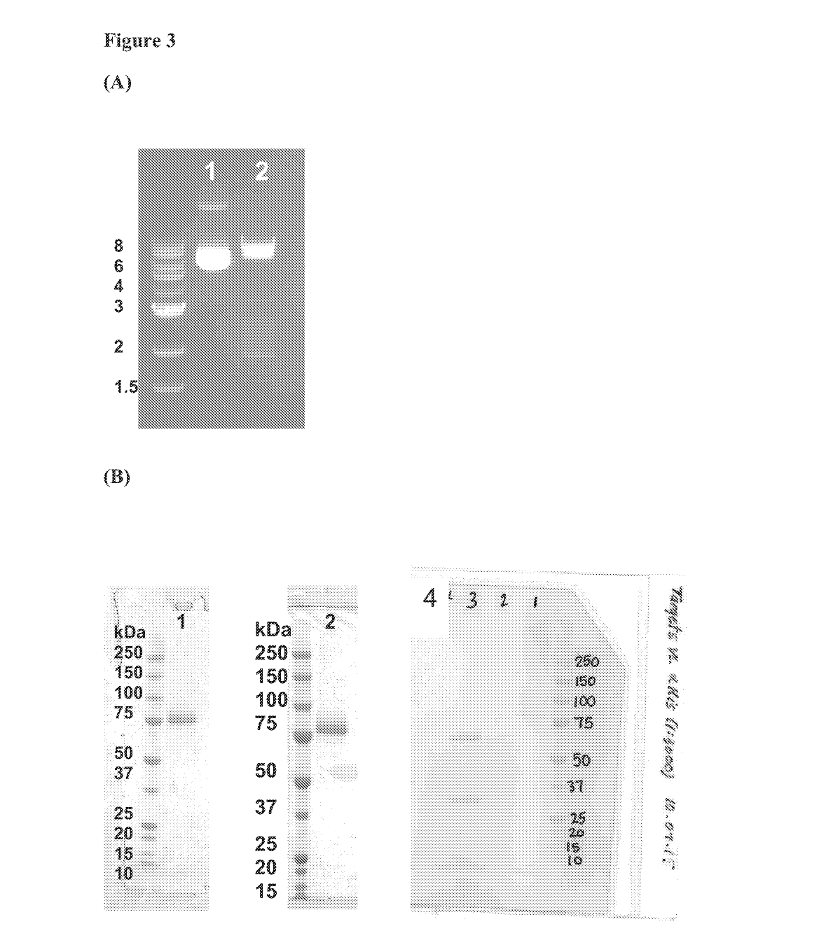

Plasmid DNA corresponding to SlpA paralogue was extracted from a culture grown from a glycerol stock using an EndoFree Giga kit from Qiagen. The identity of the plasmid DNA was confirmed by restriction digestion with AscI and HpaI restriction enzymes (see FIG. 3A).

A large scale transfection (300 ml) was performed in HEK293F cells for large scale expression of SlpA paralogue protein. A total of 3×10⁸ cells were transfected with 300 μg of SlpA paralogue plasmid DNA. The supernatant was harvested by centrifugation (3000 rpm for 15 min at room temperature) at 3 days and 7 days post-transfection. The transfected supernatant was filtered through a 0.22 μm filter and purified on a Ni column (HisTRAP HP, GE Healthcare) using the AktaPurifier FPLC (see Table 3 for FPLC procedure). The eluted protein was buffer exchanged into D-PBS and protein concentration was determined by BCA assay. A total of 14 mg of protein was purified from a 300 ml culture. The purified protein was run on SDS-PAGE for size determination and also transferred to a nitrocellulose membrane, which was probed with an anti-His-tag antibody to confirm that a protein of the correct size containing a His-tag had been obtained (see FIG. 3B). We found that while the protein ran at the correct size of 84 kDa, it would not bind the anti-His-tag antibody, which could be due to the folding of the protein. Mass spectrometry is used to confirm the identity of the protein.

### Example 4

### Expression and Purification of C. difficile CD1021 Protein

A C. difficile CD1021 nucleic acid sequence from was obtained from the NCBI public database (accession number: AM180355 (region: 1191725-1193632; see, also, WO2009/108652A1). Using standard molecular biological methods, the signal peptide and transmembrane regions of the CD1021 gene were removed and an HAVT20 leader sequence, His-tags, and Kozak sequence were added before cloning the construct into the pcDNA3002Neo plasmid using AscI and HpaI restriction enzyme sites (SEQ ID NO:26). The nucleic acid sequence of CD1021 was subsequently codon optimized for mammalian cell expression.

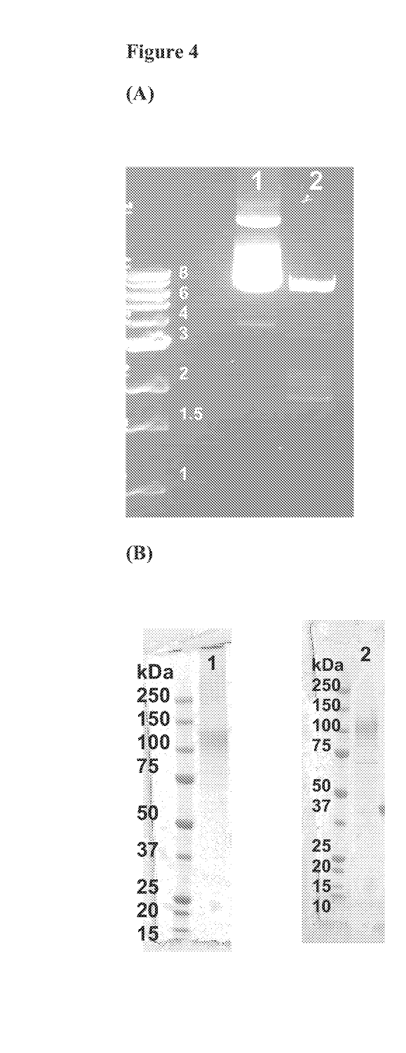

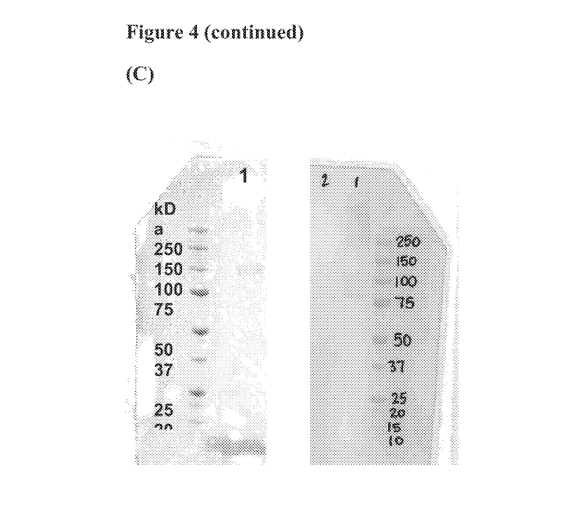

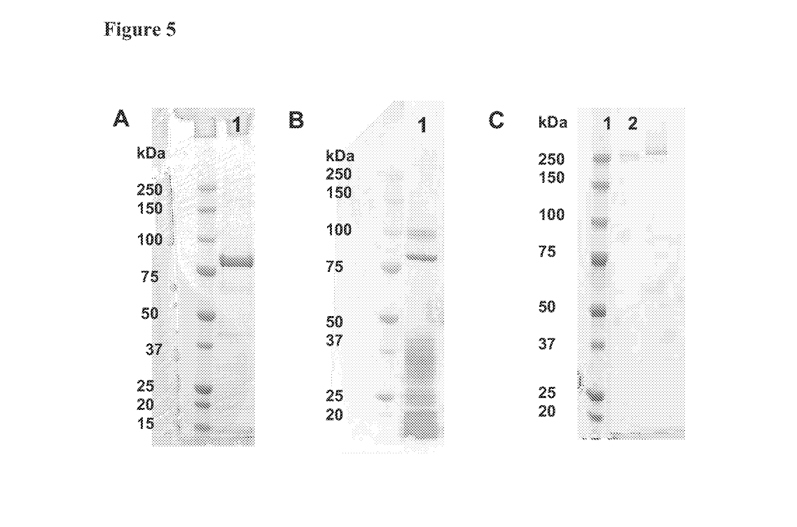

Plasmid DNA corresponding to CD1021 was extracted from a culture grown from a glycerol stock using an EndoFree Giga kit from Qiagen. The identity of the plasmid DNA was confirmed by restriction digestion with AscI and HpaI restriction enzymes (see FIG. 4A).

A large scale transfection (300 ml) was performed in HEK293F cells for large scale expression of CD1021 protein. A total of 3×10⁸ cells were transfected with 300 μg of CD1021 plasmid DNA. The supernatant was harvested by centrifugation (3000 rpm for 15 min at room temperature) at 3 days and 7 days post-transfection. The transfected supernatant was filtered through a 0.22 μm filter and purified on a Ni column (HisTRAP HP, GE Healthcare) using the AktaPurifier FPLC (see Table 3 for FPLC procedure). The eluted protein was buffer exchanged into D-PBS and protein concentration was determined by BCA assay. A total of 10 mg of protein was purified from a 300 ml culture. The purified protein was run on SDS-PAGE for size determination and also transferred to a nitrocellulose membrane, which was probed with an anti-His-tag antibody to confirm that a protein of the correct size containing a His-tag had been obtained (see FIG. 4C). We found that a protein of larger than predicted size was obtained, which is likely due to the protein having been glycosylated by expression within mammalian cells. Mass spectrometry is used for further confirmation of the identity of the protein.

### Example 5

### Expression and Purification of C. difficile FliD Protein

The FliD gene was taken from C. difficile strain R20291 and was determined to be 88% conserved among several strains (ATCC43255, 630, and CD196). Using standard molecular biological methods, the signal peptide and transmembrane regions of the FliD gene were removed and the HAVT20 leader sequence, His-tags and Kozak sequence were added before the sequence was cloned into the pcDNA3002Neo plasmid using AscI and HpaI restriction sites (SEQ ID NO:30). The nucleic acid sequence of FliD was subsequently codon optimized for mammalian cell expression.

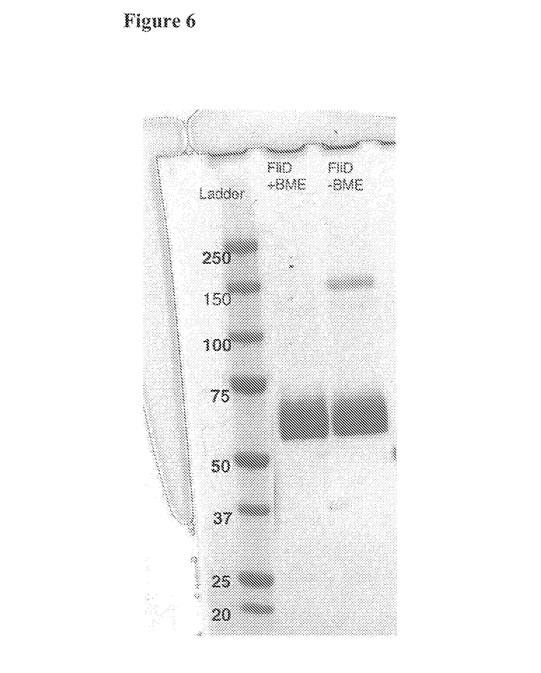

The plasmid DNA was extracted from a culture grown from a glycerol stock. The plasmid DNA was extracted using an EndoFree Giga kit from Qiagen. A large scale transfection (300 ml) was performed in HEK293F cells to obtain a large quantity of FliD protein. A total of 3×10⁸ cells were transfected with 300 μg of FliD plasmid DNA. The supernatant was harvested by centrifugation (3000 rpm for 15 min at room temperature) at 3 days and 7 days post-transfection. The supernatant from the transfected cells was filtered through a 0.22 μm filter and passed over a Ni column (HisTRAP HP, GE Healthcare) using the AktaPurifier FPLC (see Table 3 for FPLC procedure). The eluted protein was buffer exchanged into D-PBS and the concentration determined by BCA assay. A total of 68 mg was purified from a 300 ml culture. The purified protein was run on an SDS-PAGE gel to confirm its size (see FIG. 6). The protein was predicted to be 55 kDa, however, it ran at a larger size than expected, ˜65 kDa. The larger size could be due to the protein being glycosylated by mammalian cells or it could be due to dimerization. The protein was identified by mass spectrometry to be FliD protein.

### Example 6

### Generation of Antibodies Against C. difficile Spore Antigens in Mice

For antibody production, pairs of 5 to 12-week-old BALB/c mice (from Charles River, Wilmington, Mass. or another source) are inoculated (on day 1) subcutaneously or intraperitoneally with 2-50 μg of recombinant protein (or DNA encoding antigen via intramuscular (im) injection) in phosphate-buffered saline (PBS; pH 7.2), mixed with an equal volume of Complete Freund's Adjuvant (Difco, BD Biosciences, Oakville, ON, Canada) or another suitable adjuvant depending on the route of administration. Subcutaneous (or ip) boost injections of 2-25 μg of recombinant protein (or DNA via im injection) in PBS mixed with an equal portion of a suitable adjuvant (Incomplete Freund's Adjuvant (Difco) are given on days 21, 35 and 50. The mice are given a final boost of 0.5-5 μg of recombinant protein via ip, iv (or im for DNA) in PBS and sacrificed 3 days later.

The serum IgG response to the antigen or whole spore is monitored via enzyme-linked immunosorbent assays (ELISA) or other suitable assays using sera collected from the mice during the inoculation protocol, as described in Berry et al. (2004), using a suitable 96 well or similar plate (e.g., MaxiSorp™, Nalge-NUNC, Rochester, N.Y.). The assay plates are coated with either recombinant antigen, or as a negative control, bovine serum albumin (BSA) or another suitable protein, each at 75-1000 ng per well. Once sufficient IgG titers are detected (e.g., an OD at 405 nm in an ELISA assay of at least three-five fold above background), the mice receive a final push boost and are sacrificed. Spleens and/or lymph nodes are isolate and hybridoma production and growth is performed as described (Berry et al., 2004). Subsequent mAb harvesting, concentration and isotyping are performed as described previously (Berry et al., 2004).

Alternatively CD38+ or CD138+ lymphoblasts are isolated using single cell sorting or bulk sorting (via FACS or with appropriate columns), and recovered RNA is used for expression screening for mAbs using phage or cassettes. Immune and preimmune sera (diluted 1:2000 with 0.2% BSA in PBS) are used as positive and negative controls, respectively. The mAbs are purified using HiTrap™ Protein G HP or another suitable column according to the manufacturer's instructions (Amersham Biosciences, Uppsala, Sweden). After buffer exchange with PBS, mAb concentrations are determined with a Micro BCA Protein Assay Kit according to the manufacturer's instructions (Pierce, Rockford, Ill.). Transgenic mice can receive additional boosts to elicit high titer IgG responses, indicative of adequate B cell sensitization, as necessary.

### Example 7

### Generation of Antibodies Against C. difficile Spore Antigens in Rabbits

For antibody production, 2 rabbits undergo a prebleed at Day 0 before being immunized subcutaneously (SQ) with 50-200 μg of a recombinant protein in phosphate-buffered saline (PBS; pH 7.2), mixed with an equal volume of Complete Freund's Adjuvant. Subcutaneous boosters of 20-100 μg of recombinant protein in PBS mixed with an equal portion of Incomplete Freund's Adjuvant are given on days 28, 47 and 66. The rabbits are immunized in four different sites; 2 in the hind quarters and 2 in the scapula. Immunizations are prepared using luer-lok connectors to allow for gentle emulsification. The rabbits undergo a test bleed at Day 59 and a terminal bleed at Day 78. The terminal bleed is performed while the animal is under anesthetic.

The serum Ab response to the protein is monitored via enzyme-linked immunosorbent assays (ELISA) or other suitable assay, using sera collected from the rabbit during a test bleed, with a suitable 96 well plate (e.g., MaxiSorp™, Nalge-NUNC, Rochester, N.Y.). The plates are coated with either recombinant protein or, as a negative control, bovine serum albumin (BSA) or other protein, both at 75-1000 ng per well. Immune and preimmune sera (diluted 1:2000 with 0.2% BSA in PBS) serve as positive and negative controls, respectively. Once sufficient Ab titers are detected (an OD at 450 nm in ELISA at least three-five fold above background), the rabbits receive the final boost and undergo the terminal bleed. If the titers are not sufficient the rabbits will receive additional boosts. The pAbs are purified from the terminal bleed using a Protein A column, after which, the buffer is exchanged with PBS and the pAb concentration is determined.

### Example 8

### Testing the Protective Effect of Clostridium difficile Spore Antigens (Active Immunization) in Hamsters

Golden Syrian Hamsters (female, 6-7 weeks of age) are immunized (i.d.) twice (V1, V2, days 1 and 28 respectively) with DNA encoding spore antigens (10 μg/hamster), and once (V3, day 35) with the respective recombinant proteins (10 μg/hamster). See diagram below. Bleeds are performed after each vaccination to test antibody production (ELISA). One week after the last vaccination, hamsters are treated with clindamycin (30 mg/kg, orally). Twelve hours post antibiotic treatment, animals are challenged orogastrically with 100 spores of C. difficile B1 strain (in 0.2 ml saline) and monitored daily for clinical signs. Any animals showing irreversible moribundity are euthanized for humane reasons and remaining surviving hamsters are euthanized 7 days post challenge. Protection is evaluated by clinical signs, survival rates, and by determining the number of spores recovered in the cecum at the time of euthanasia. Protective antigens are predicted to cause a reduction in the number of recovered spores, as well as, in spore shedding over the course of days, and result in improved survival.

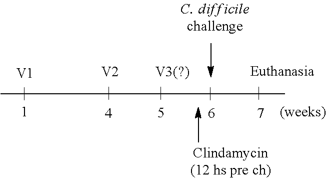

### Example 9

### Testing the Protective Effect of Antibodies Against Clostridium Difficile Spore Antigens in Hamsters

A. Primary Challenge Model

To test the protective capabilities of mAbs to spore antigens, hamsters are treated with the antibodies (50 mg/kg/day) delivered i.p. singly or in combination for a total of 4 days (72, 48, 24, and 0 h prior to the administration of C. difficile spores). Animals are injected intraperitoneally with clindamycin 12 hours prior to the orogastric delivery of 100 C. difficile strain B1 spores. Hamsters are observed for mortality daily until all hamsters have either succumbed to disease or become free of disease symptoms. When antibodies are provided singly they are predicted to increase survival by 50% and this protection can wane after day 5 (20%). Antibody treatment is also predicted to reduce CFU in the feces at the time of necropsy by 1 log. Moreover, combination therapy is predicted to result in increased protection to 95% at day 2, as well as significant protection throughout the study (50%), with a 2 log reduction of CFU.

B. Relapse Model

Treatments with antibiotics to eliminate C. difficile kill the vegetative bacteria but leave the spores behind. This is the main problem underlying recurrent infections, in which patients have episodes of CDAD between antibiotic treatments. To determine whether the antibodies can prevent mortality in a relapse situation, the hamster relapse model can be employed. (Babcock et al., 2009). In this model, hamsters are treated with vancomycin which protects from C. difficile disease, but when vancomycin treatment is discontinued, hamsters relapse with disease. Hamsters are given clindamycin as above, and 12 hours later they are orogastrically challenged with C. difficile strain B1 spores (100,000 CFU). Vancomycin (10 mg/kg/day) is provided on the day of spore challenge and daily for two subsequent days. Hamsters are treated with combinations of mAbs (50 mg/kg/day) on days 2 to 6 following spore challenge. Treatment with the combinations is predicted to prevent relapse in 70% of the hamsters compared to 40% of those receiving vancomycin alone. Treatment is also predicted to result in reduction of bacterial shedding (2 logs vs 1 log in the vancoumycin alone group). Survival is also predicted to be improved when the mAbs are used individually, although less significantly with 45% survival, and 1 log reduction of CFU recovered in feces.

### Example 10

### Immunization of Mice with Spore Antigen to Produce mAbs

Mice were immunized with spore antigens to produce mAbs. Each antigen group had 4 mice which were immunized/boosted i.p. with 10 μg/mouse of purified antigen in 70% PBS+30% Emulsigen with 5 μg/mouse CpG. The mice were given 3 boosts, one per week following the initial immunization. The mice were given a final boost (4 μg/mouse in PBS) before the terminal bleed. The sera containing mAbs against the spore antigens are then characterized.

### Example 11

### In Vitro Spore Antigen mAb Characterization

1) Detection ELISA. This assay was performed to test the binding of antibodies from mice immunized with C. difficile spore antigens to spore antigens and whole spores. Anti-C. difficile spore (ATCC 43255) polyclonal antibody is used as a positive control for the assay.

a) Whole spore ELISA. An aliquot of spores was thawed and diluted in coating buffer to a concentration of 10⁵ spores/ml. A volume of 100 μl of spores (10⁴ spores/well) was added to each well of a 96-well ELISA plate. The plate was sealed and left at room temperature overnight.

The next day the plate was washed 3 times using 300 μl/well of PBST per wash to remove any spores that are unattached. The sealed plate was blocked using 5% skim milk in PBS pH 7.4 (300 μl/well) for 1.5 hours at 37° C. After blocking, the plate was washed 3 times using 300 μl/well of PBST per wash to remove the blocking buffer. The primary antibody (mouse sera from immunized mice) was serially diluted 1:2 starting at a dilution of 1/100. The anti-C. difficile spore polyclonal Ab (pAb) used as a positive control was diluted to 1/1000. The antibody dilutions were loaded into the appropriate wells of the plate (100 μl/well). The plate was sealed and left to incubate for 1 hour at 37° C. After 1° Ab incubation, the plate was washed 3 times using 300 μl/well of PBST per wash to remove unbound 1° Ab. An appropriate secondary antibody was used at the recommended manufacturer's dilution and loaded into the appropriate wells of the plate (100 μl/well) to detect any bound 1° Ab. The plate was sealed and left to incubate for 1 hour at 37° C. After 2° Ab incubation, the plate was washed 3 times using 300 μl/well of PBST per wash to remove unbound 2° Ab. To detect any bound antibody, a peroxidase substrate was loaded into each well (100 μl/well) and left to incubate in the dark at room temperature for 10-30 minutes. The reaction was stopped using stop solution after incubation (50 μl/well) and the plate was read at 450 nm.

Results:

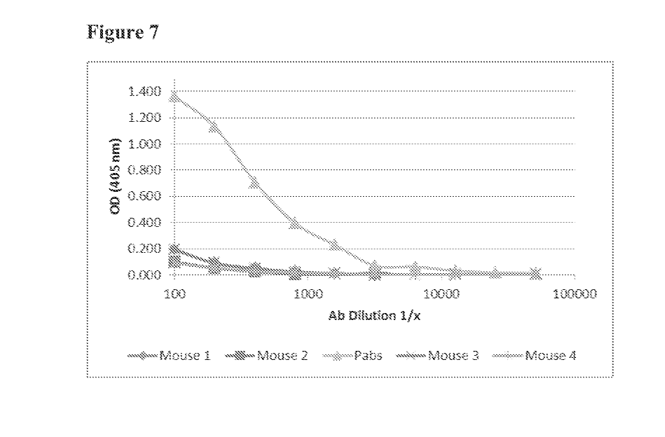

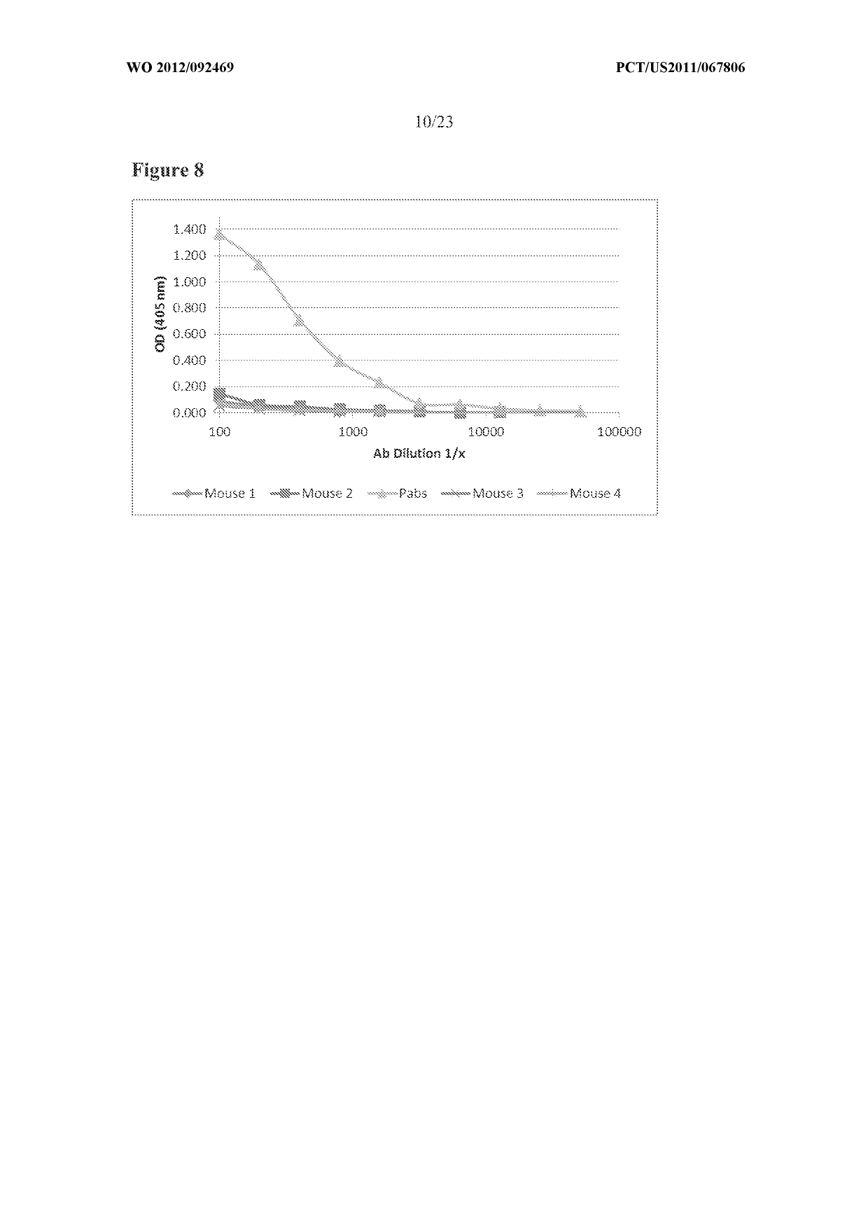

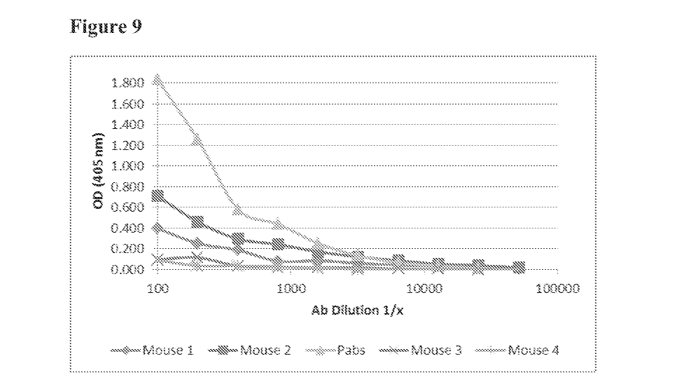

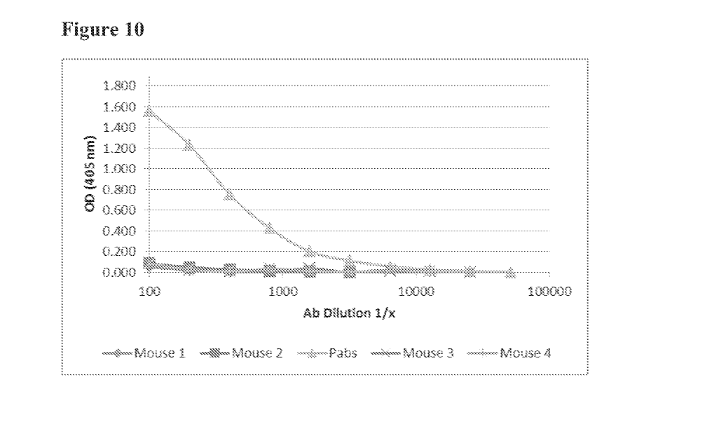

The whole spore ELISA showed that the various spore antibodies bound to isolated C. difficile spore strain ATCC 43255, as shown in FIGS. 7, 8, 9 and 10.

b) Spore Antigen ELISA. The spore antigen was diluted in coating buffer to a concentration of 0.03 μg/μL. A volume of 100 μl of the dilution was added to each well of a 96-well ELISA plate. The plate was sealed and left at room temperature overnight.

The next day the plate was washed 3 times using 300 μl/well of PBS per wash to remove any unbound antigen. The sealed plate was blocked using 1% BSA (300 μl/well) for at least 1.5 hours at room temperature. After blocking, the plate was washed 3 times using 300 μl/well of PBS per wash to remove the blocking buffer. The primary antibody (mouse sera from immunized mice) was serially diluted 1:2 starting at a dilution of 1/50. The anti-C. difficile spore polyclonal Ab (pAb) used as a positive control was serially diluted 1:2 starting at a dilution of 1/50. The antibody dilutions were loaded into the appropriate wells of the plate (100 μl/well). The plate was sealed and left to incubate for at least 1 hour at room temperature. After 1° Ab incubation, the plate was washed 3 times using 300 μl/well of PBS per wash to remove unbound 1° Ab. An appropriate secondary antibody was used at the recommended manufacturer's dilution and loaded into the appropriate wells of the plate (100 μl/well) to detect any bound 1° Ab. The plate was sealed and left to incubate for at least 1 hour at room temperature. After 2° Ab incubation, the plate was washed 3 times using 300 μl/well of PBST per wash to remove unbound 2° Ab. To detect any bound antibody, alkaline phosphatase substrate was loaded into each well (100 μl/well) and left to incubate in the dark at room temperature for at least 1 hour. The plate was read at 405 nm.

Results:

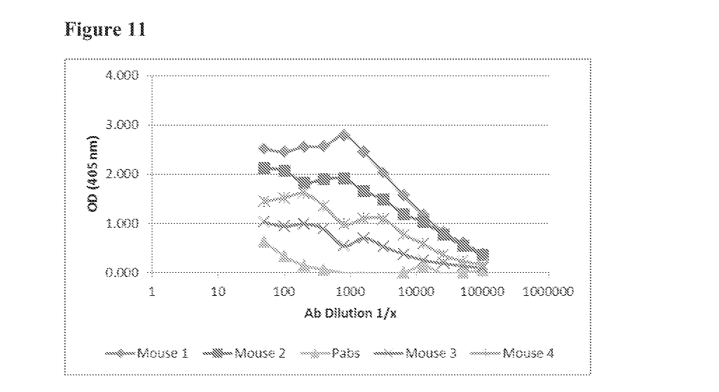

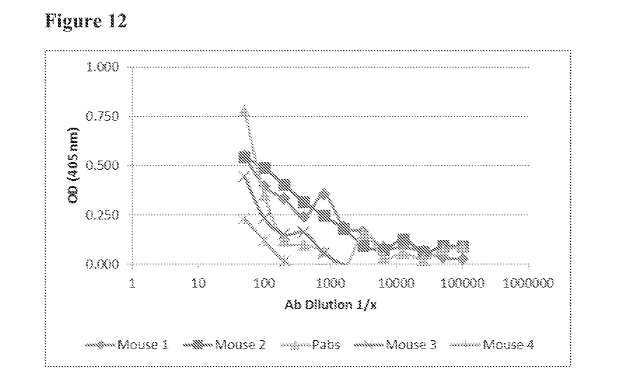

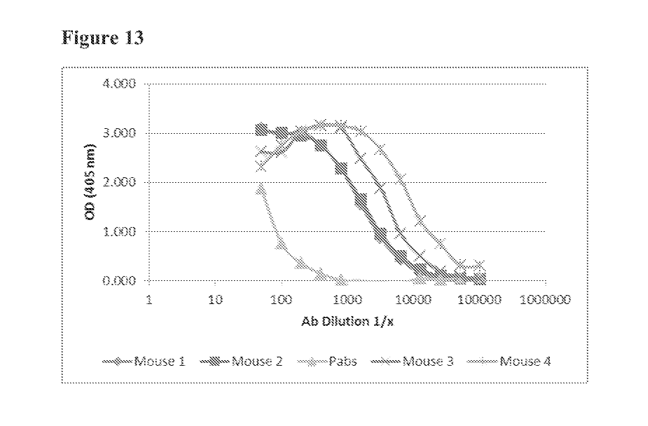

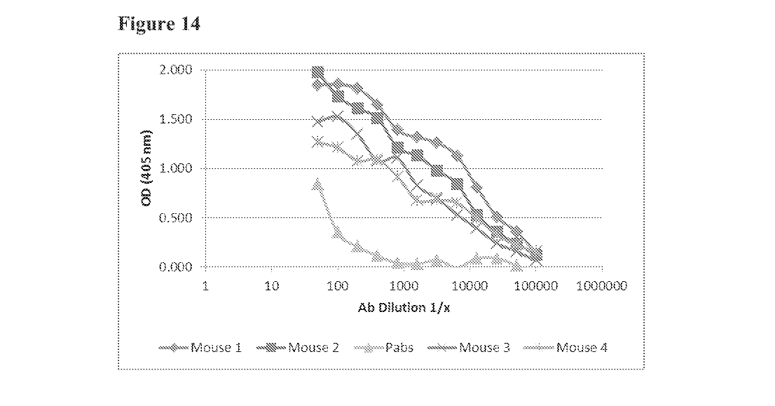

The results from the spore antigen ELISA indicate that the spore antibodies produced in mice bind to purified C. difficile spore antigens, as shown in FIGS. 11 to 14.

2) Germination Assay. This assay was performed to screen sera obtained from mice immunized with C. difficile spore antigens for inhibition of spore germination. The premise of the assay is that O.D. readings taken should decrease with time when the spores are germinating. If the antibodies in the sera inhibit germination there should be a slower decrease in O.D. over time compared to untreated spores. Anti-C. difficile spore (ATCC 43255) polyclonal antibody is used as a positive control for the assay.

The spore suspension (10⁷ spores/treatment) was prepared using recently purified spores and was heat activated in a 60° C. water bath for 20 minutes and then cooled to room temperature. The spores are sonicated for 2 minutes to break up any clumps. A volume of 200 IA of the suspension was transferred to a new tube and 1 μl of pAb was added. The tube was incubated on ice for 30 minutes. Germination media (800 μl of BHIT-G) was then added to the tube and the contents were transferred to a cuvette. The cuvettes are read (O.D. @ 600 nm) every 10 minutes over an hour period. Between readings the cuvettes are incubated at 37° C. on a shaker (50 rpm).

Results:

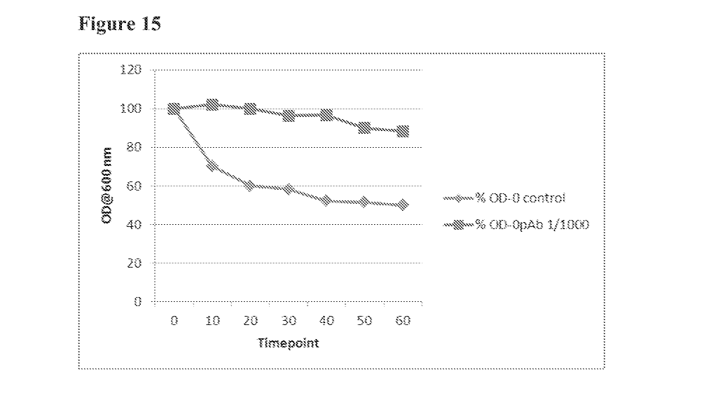

The germination assay with the pAbs show that antibodies that recognize spores can delay the onset of germination (FIG. 15).

### Example 12

### Western Blot Testing

Western blots were performed to test the recognition of antibodies from mice immunized with C. difficile spore antigens to proteins expressed on the spore surface.

The protein extracts were prepared from ATCC 43255 spores by using SDS extraction buffer and urea extraction buffer. The protein extracts were run on two 12% SDS-PAGE gels along with a mixture of four recombinant spore antigen proteins. One gel was stained with Coomassie blue to visualize the protein bands; another gel was transferred to nitrocellulose membrane and blotted with anti-whole spore polyclonal Ab. The urea extracts were run on separate SDS-PAGE gels; each individual gel was blotted with sera from mouse immunized with different spore antigens.

a) Protein Extraction. ATCC 43255 spores (3×10⁷) were washed with PBS and resuspended with 1 mL SDS extraction buffer (62.5 mM Tris-HCl, pH 6.8; 25% glycerol; 2% SDS; 5% β-mercaptoethanol and 0.01% Bromophenol Blue); the sample was boiled for 15 mins, and was passed through 0.2 μm filter to remove the spores.

ATCC 43255 spores (3×10⁷) were washed with PBS and resuspended with 1 mL urea extraction buffer (8M Urea and 10%β-mercaptoethanol in 50 mM Tris-HCl); the sample was incubated at 30° C. for 2 hours with vortex every 10 mins, and was passed through a 0.2 μm filter to remove the spores.

b) Western blot: i) Transfer: Presoaked filter pads, nitrocellulose and Whatman paper were place for 20 minutes in 1× transfer buffer. The gel was equilibrated for 5 minutes with the filter pads, nitrocellulose, and Whatman paper in 1× transfer buffer. The transfer was run at 2-8° C. for 1 hour at 100 V. ii) Staining: The membrane was blocked with 5% skim milk at room temperature for 1 hr. The membrane was washed for 3×10 minutes in TBS-T at room temperature. The membrane was placed protein side up into a container with 20 mL of 1° antibody (1:1000) solution and incubated at 2-8° C. for 18-24 hours. The membrane was washed for 3×10 minutes in TBS-T at room temperature. Then the membrane was then placed protein side up into a container with 20 mL of 2° antibody (1:10000) solution and incubated at room temperature for 2 hours. The membrane was washed for 3×10 minutes in TBS-T at room temperature. iii) Detection: SIGMAFAST™ BCIP®/NBT tablets were removed from freezer and warmed to room temperature. 2 tablets were placed in 20 mL (2×) of LW and vortexed until dissolved. The membrane was incubated with SIGMAFAST™ BCIP®/NBT for approximately 30 seconds or until the desired intensity is reached. The membrane was then washed with copious amounts of LW to prevent overstaining. The membrane was allowed to dry and stored away from light for future reference

Results:

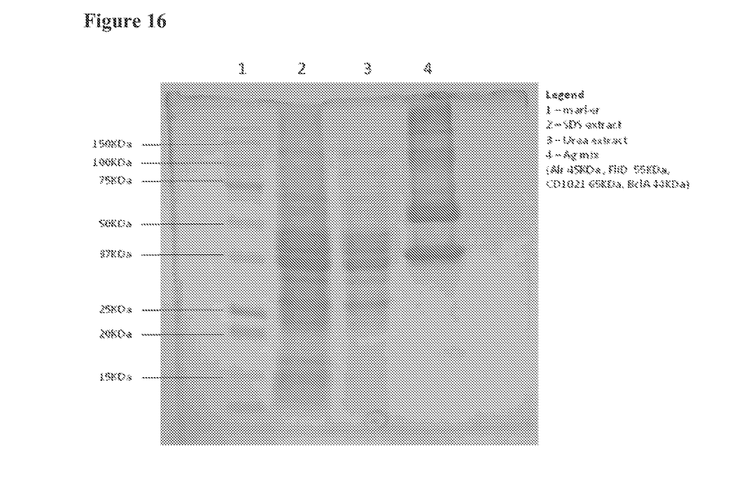

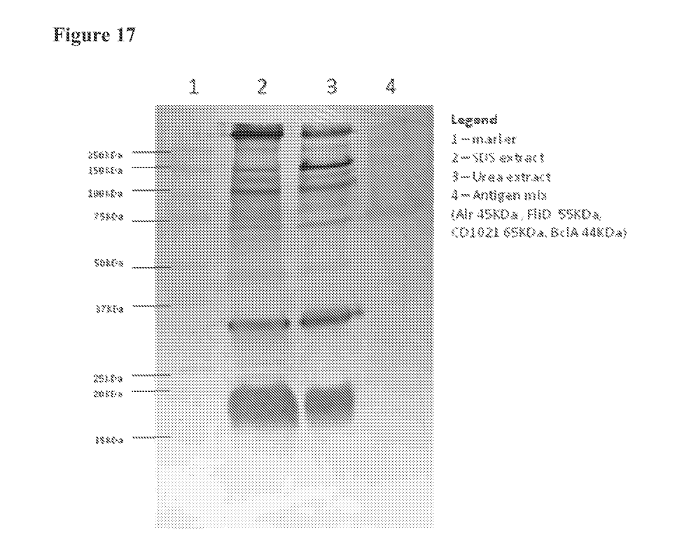

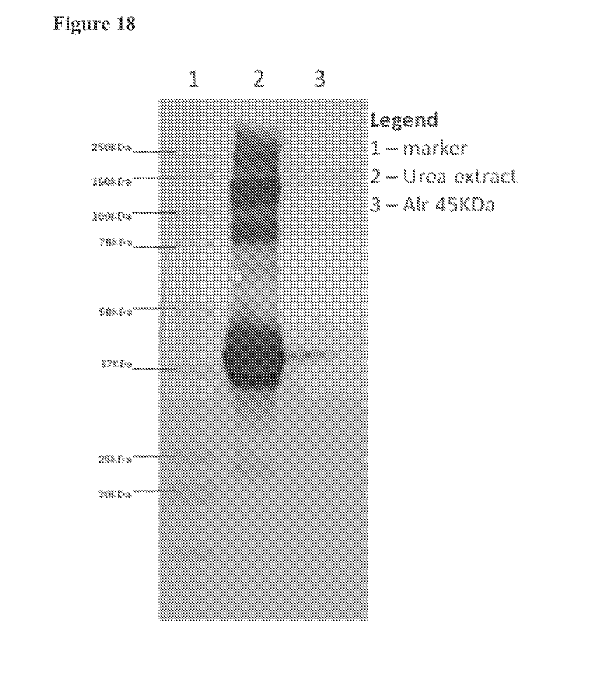

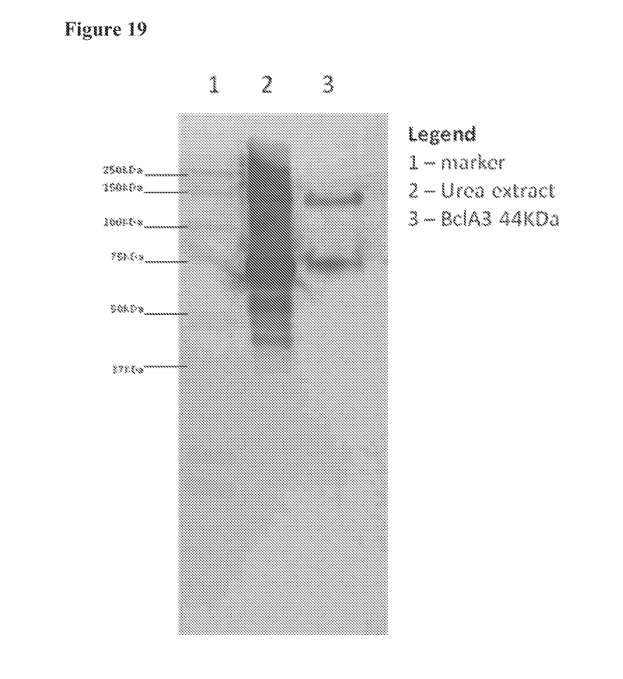

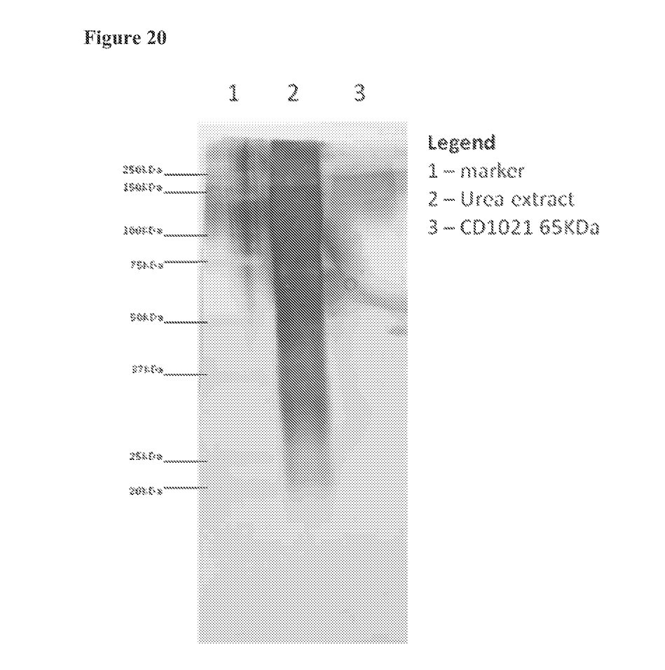

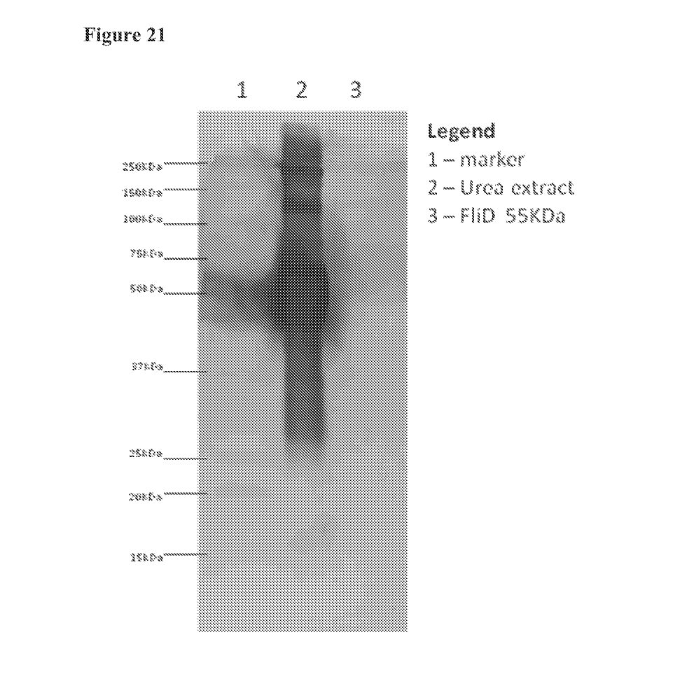

The Western blots showed that the antibodies made in mice immunized with C. difficile spore antigens recognized spore proteins as shown in FIGS. 16 to 21.

While specific aspects of the invention have been described and illustrated, such aspects should be considered illustrative of the invention only and not as limiting the invention as construed in accordance with the accompanying claims.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

1. An isolated antibody or fragment thereof that binds to a C. difficile spore polypeptide or fragment thereof, wherein the C. difficile spore polypeptide is selected from the group consisting of BclA1, BclA2, BclA3, Alr, SlpA paralogue, SlpA HMW, CD1021, IunH, Fe-Mn-SOD, and FliD.

2. An isolated antibody or fragment thereof that binds to a C. difficile spore polypeptide or fragment thereof, wherein the C. difficile spore polypeptide or fragment thereof comprises an amino acid sequence at least 80-95% identical to the amino acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

3. A composition comprising the antibody or fragment thereof of claim 1.

4. A composition comprising the antibody or fragment thereof of claim 2.

5. The antibody or fragment thereof according to anyone of claims 1 and 2, wherein the antibody is a polyclonal antibody or a monoclonal antibody.

6. (canceled)

7. The antibody or fragment thereof according to anyone of claims 1 and 2, wherein the antibody or fragment thereof is a human antibody.

8. The antibody or fragment thereof according to anyone of claims 1 and 2, wherein the antibody or fragment thereof is selected from the group consisting of: (a) a whole immunoglobulin molecule; (b) an scFv; (c) a chimeric antibody; (d) a Fab fragment; (e) an F(ab′)2; and (f) a disulfide linked Fv.

9. The antibody or fragment thereof according to anyone of claims 1 and 2, which comprises a heavy chain immunoglobulin constant domain selected from the group consisting of: (a) a human IgM constant domain; (b) a human IgG1 constant domain; (c) a human IgG2 constant domain; (d) a human IgG3 constant domain; (e) a human IgG4 constant domain; and (f) a human IgAlI2 constant domain.

10. The antibody or fragment thereof according to anyone of claims 1 and 2, which comprises a light chain immunoglobulin constant domain selected from the group consisting of: (a) a human Ig kappa constant domain; and (b) a human Ig lambda constant domain.

11. The antibody or fragment thereof according to anyone of claims 1 and 2, wherein the antibody or fragment thereof binds to an antigen with an affinity constant (K_{aff}) of at least 1×10⁹ M.

12. (canceled)

13. The composition according to anyone of claims 3 and 4, further comprising an antibody that binds to C. difficile toxin A, an antibody that binds to C. difficile toxin B, or a combination of antibodies that bind toxin A and toxin B.

14. The composition according to anyone of claims 3 and 4, further comprising an antibiotic.

15. The composition of claim 14, wherein the antibiotic is metronidazole or vanomycin.

16. A method of treatment of C. difficile associated disease or passive immunization comprising the step of administering to a subject the composition according to anyone of claims 3 and 4.

17-71. (canceled)

72. The antibody or fragment thereof according to anyone of claims 1 and 2, wherein the antibody or fragment thereof inhibits or delays spore germination.


SEQUENCE LISTING (30 sequences)

SEQ ID NO: 1 - protein, 239 aa, Clostridium difficile
MACPGFLWALVISTCLEFSMAMRKIILYLNDDTFISKKYPDKNFSNLDYCLIGSKCSNSFVKEKLITFFKVRIPDILKDKSILKAELFIHIDSNKNHIFKEKVDIEIKRISEYYNLRTITWNDRVSMENIRGYLPIGISDTSNYICLNITGTIKAWAMNKYPNYGLALSLNYPYQIFEFTSSRDCNKPYILVTFEDRIIDNCYPKCECLPIRITGPMGPRGATGSIGPMGATGPTGATG

SEQ ID NO: 2 - protein, 467 aa, Clostridium difficile
MACPGFLWALVISTCLEFSMAMSDISGPSLYQDVGPTGPTGATGPTGPTGPRGATGATGANGITGPTGNTGATGANGITGPTGNMGATGANGTTGSTGPTGNTGATGANGITGPTGATGATGANGITGPTGNKGATGANGITGPTGATGATGANGITGPTGNTGATGANGATGLTGATGATGANGITGPTGATGATGANGVTGATGPTGNTGATGPTGSIGATGANGVTGATGPIGATGPTGAVGATGPDGLVGPTGPTGPTGATGANGLVGPTGPTGATGANGLVGPTGATGATGVAGAIGPTGAVGATGPTGADGAVGPTGATGATGANGATGPTGAVGATGANGVAGPIGPTGPTGANGVAGATGATGATGANGATGPTGAVGATGANGVAGPIGPTGPTGANGTTGATGATGATGANGATGPTGATGATGVLAANNAQFTVSSSSLGNNTLVTFNSSFINGTN

SEQ ID NO: 3 - protein, 525 aa, Clostridium difficile
MACPGFLWALVISTCLEFSMAMSRNKYFGPFDDNDYNNGYDKYDDCNNGRDDYNSCDCHHCCPPSCVGPTGPMGPRGRTGPTGPTGPTGPGVGGTGPTGPTGPTGPTGNTGNTGATGLRGPTGATGGTGPTGATGAIGFGVTGPTGPTGPTGATGATGADGVTGPTGPTGATGADGITGPTGATGATGFGVTGPTGPTGATGVGVTGATGLIGPTGATGTPGATGPTGAIGATGIGITGPTGATGATGADGATGVTGPTGPTGATGADGVTGPTGATGATGIGITGPTGATGATGIGITGATGLIGPTGATGATGATGPTGVTGATGAAGLIGPTGATGVTGADGATGATGATGATGPTGADGLVGPTGATGATGADGLVGPTGPTGATGVGITGATGATGATGPTGADGLVGPTGATGATGADGVAGPTGATGATGNTGADGATGPTGATGPTGADGLVGPTGATGATGLAGATGATGPIGATGPTGADGATGATGATGPTGADGLVGPTGATGATGATGPTGP

SEQ ID NO: 4 - protein, 406 aa, Clostridium difficile
MACPGFLWALVISTCLEFSMAMQKITVPTWAEINLDNLRFNLNNIKNLLEEDIKICGVIKADAYGHGAVEVAKLLEKEKVDYLAVARTAEGIELRQNGITLPILNLGYTPDEAFEDSIKNKITMTVYSLETAQKINEIAKSLGEKACVHVKIDSGMTRIGFQPNEESVQEIIELNKLEYIDLEGMFTHFATADEVSKEYTYKQANNYKFMSDKLDEAGVKIAIKHVSNSAAIMDCPDLRLNMVRAGIILYGHYPSDDVFKDRLELRPAMKLKSKIGHIKQVEPGVGISYGLKYTTTGKETIATVPIGYADGFTRIQKNPKVLIKGEVFDVVGRICMDQIMVRIDKDIDIKVGDEVILFGEGEVTAERIAKDLGTINYEVLCMISRRVDRVYMENNELVQINSYLLK

SEQ ID NO: 5 - protein, 620 aa, Clostridium difficile
MACPGFLWALVISTCLEFSMAAETTQVKKETITKKEATELVSKVRDLMSQKYTGGSQVGQPIYEIKVGETLSKLKIITNIDELEKLVNALGENKELIVTITDKGHITNSANEVVAEATEKYENSADLSAEANSITEKAKTETNGIYKVADVKASYDSAKDKLVITLRDKTDTVTSKTIEIGIGDEKIDLTANPVDSTGTNLDPSTEGFRVNKIVKLGVAGAKNIDDVQLAEITIKNSDLNTVSPQDLYDGYRLTVKGNMVANGTSKSISDISSKDSETGKYKFTIKYTDASGKAIELTVESTNEKDLKDAKAALEGNSKVKLIAGDDRYATAVAIAKQTKYTDNIVIVNSNKLVDGLAATPLAQSKKAPILLASDNEIPKVTLDYIKDIIKKSPSAKIYIVGGESAVSNTAKKQLESVTKNVERLAGDDRHMTSVAVAKAMGSFKDAFVVGAKGEADAMSIAAKAAELKAPIIVNGWNDLSADAIKLMDGKEIGIVGGSNNVSSQIENQLADVDKDRKVQRVEGETRHDTNAKVIETYYGKLDKLYIAKDGYGNNGMLVDALAAGPLAAGKGPILLAKADITDSQRNALSKKLNLGAEVTQIGNGVELTVIQKIAKILGW

SEQ ID NO: 6 - protein, 479 aa, Clostridium difficile
MGKTAQDLAKKYVFNKTDLNTLYRVLNGDEADTNRLVEEVSGKYQVVLYPEGKRVTTKSAAKASIADENSPVKLTLKSDKKKDLKDYVDDLRTYNNGYSNAIEVAGEDRIETAIALSQKYYNSDDENAIFRDSVDNVVLVGGNAIVDGLVASPLASEKKAPLLLTSKDKLDSSVKAEIKRVMNIKSTTGINTSKKVYLAGGVNSISKEVENELKDMGLKVTRLAGDDRYETSLKIADEVGLDNDKAFVVGGTGLADAMSIAPVASQLRNANGKMDLADGDATPIVVVDGKAKTINDDVKDFLDDSQVDIIGGENSVSKDVENAIDDATGKSPDRYSGDDRQATNAKVIKESSYYQDNLNNDKKVVNFFVAKDGSTKEDQLVDALAAAPVAANFGVTLNSDGKPVDKDGKVLTGSDNDKNKLVSPAPIVLATDSLSSDQSVSISKVLDKDNGENLVQVGKGIATSVINKLKDLLSMLEGT

SEQ ID NO: 7 - protein, 601 aa, Clostridium difficile
MACPGFLWALVISTCLEFSMATSSNKSVDLYSDVYIEKYFNRDKVMEVNIEIDESDLKDMNENAIKEEFKVAKVTVDGDTYGNVGIRTKGNSSLISVANSDSDRYSYKINFDKYNTSQSMEGLTQLNLNNCYSDPSYMREFLTYSICEEMGLATPEFAYAKVSINGEYHGLYLAVEGLKESYLENNFGNVTGDLYKSDEGSSLQYKGDDPESYSNLIVESDKKTADWSKITKLLKSLDTGEDIEKYLDVDSVLKNIAINTALLNLDSYQGSFAHNYYLYEQDGVFSMLPWDFNMSFGGFSGFGGGSQSIAIDEPTTGNLEDRPLISSLLKNETYKTKYHKYLEEIVTKYLDSDYLENMTTKLHDMIASYVKEDPTAFYTYEEFEKNITSSIEDSSDNKGFGNKGFDNNNSNNSDSNNNSNSENKRSGNQSDEKEVNAELTSSVVKANTDNETKNKTTNDSESKNNTDKDKSGNDNNQKLEGPMGKGGKSIPGVLEVAEDMSKTIKSQLSGETSSTKQNSGDESSSGIKGSEKFDEDMSGMPEPPEGMDGKMPPGMGNMDKGDMNGKNGNMNMDRNQDNPREAGGFGNRGGGSVSKTTTYFK

SEQ ID NO: 8 - protein, 322 aa, Clostridium difficile
MEKRKVIIDCDPGIDDSLAILLALNSPELEVIGITTCCGNVPANIGAENALKTLQMCSSLNIPVYIGEEAPLKRKLVTAQDTHGEDGIGENFYQKVVGAKAKNGAVDFIINTLHNHEKVSIIALAPLTNIAKALIKDKKAFENLDEFVSMGGAFRIHGNCSPVAEFNYWVDPHGADYVYKNLSKKIHMVGLDVTRKIVLTPNIIEFINRLDKKMAKYITEITRFYIDFHWEQEGIIGCVINDPLAVAYFIDRSICKGFESYVEVVEDGIAMGQSIVDSFNFYKKNPNAIVLNEVDEKKFMYMFLKRLFKGYEDIIDSVEGVI

SEQ ID NO: 9 - protein, 234 aa, Clostridium difficile
MKKKILIPVIMSLFIISQCITSFAFTPENNKFKVKPLPYAYDALEPYIDKETMKLHHDKHYQAYVDKLNAALEKYPELYNYSLCELLQNLDSLPKDIATTVRNNAGGAYNHKFFFDIMTPEKTIPSESLKEAIDRDFGSFEKFKQEFQKSALDVFGSGWAWLVATKDGKLSIMTTPNQDSPVSKNLTPIIGLDVWEHAYYLKYQNRRNEYIDNWFNVVNWNGALENYKNLKSQD

SEQ ID NO: 10 - protein, 507 aa, Clostridium difficile
MSSISPVRVTGLSGNFDMEGIIEASMIRDKEKVDKAKQEQQIVKWKQEIYRNVIQESKDLYDKYLSVNSPNSIVSEKAYSSTRITSSDESIIVAKGSAGAEKINYQFAVSQMAEPAKFTIKLNSSEPIVRQFPPNASGASSLTIGDVNIPISEQDTTSTIVSKINSLCADNDIKASYSEMTGELIISRKQTGSSSDINLKVIGNDNLAQQIANDNGITFANDASGNKVASVYGKNLEADVTDEHGRVTHISKEQNSFNIDNIDYNVNSKGTAKLTSVTDTEEAVKNMQAFVDDYNKLMDKVYGLVTTKKPKDYPPLTDAQKEDMTTEEIEKWEKKAKEGILRNDDELRGFVEDIQSAFFGDGKNIIALRKLGINESENYNKKGQISFNADTFSKALIDDSDKVYKTLAGYSSNYDDKGMFEKLKDIVYEYSGSSTSKLPKKAGIEKTASASENVYSKQIAEQERNISRLVEKMNDKEKRLYAKYSALESLLNQYSSQMNYFSQAQGN

SEQ ID NO: 11 - protein, 534 aa, Artificial Sequence
  Description of Artificial Sequence Synthetic polypeptide
AATMACPGFLWALVISTCLEFSMAMSRNKYFGPFDDNDYNNGYDKYDDCNNGRDDYNSCDCHHCCPPSCVGPTGPMGPRGRTGPTGPTGPTGPGVGGTGPTGPTGPTGPTGNTGNTGATGLRGPTGATGGTGPTGATGAIGFGVTGPTGPTGPTGATGATGADGVTGPTGPTGATGADGITGPTGATGATGFGVTGPTGPTGATGVGVTGATGLIGPTGATGTPGATGPTGAIGATGIGITGPTGATGATGADGATGVTGPTGPTGATGADGVTGPTGATGATGIGITGPTGATGATGIGITGATGLIGPTGATGATGATGPTGVTGATGAAGLIGPTGATGVTGADGATGATGATGATGPTGADGLVGPTGATGATGADGLVGPTGPTGATGVGITGATGATGATGPTGADGLVGPTGATGATGADGVAGPTGATGATGNTGADGATGPTGATGPTGADGLVGPTGATGATGLAGATGATGPIGATGPTGADGATGATGATGPTGADGLVGPTGATGATGATGPTGPHHHHHH

SEQ ID NO: 12 - protein, 418 aa, Artificial Sequence
  Description of Artificial Sequence Synthetic polypeptide
IRRAATMACPGFLWALVISTCLEFSMAMQKITVPTWAEINLDNLRFNLNNIKNLLEEDIKICGVIKADAYGHGAVEVAKLLEKEKVDYLAVARTAEGIELRQNGITLPILNLGYTPDEAFEDSIKNKITMTVYSLETAQKINEIAKSLGEKACVHVKIDSGMTRIGFQPNEESVQEIIELNKLEYIDLEGMFTHFATADEVSKEYTYKQANNYKFMSDKLDEAGVKIAIKHVSNSAAIMDCPDLRLNMVRAGIILYGHYPSDDVFKDRLELRPAMKLKSKIGHIKQVEPGVGISYGLKYTTTGKETIATVPIGYADGFTRIQKNPKVLIKGEVFDVVGRICMDQIMVRIDKDIDIKVGDEVILFGEGEVTAERIAKDLGTINYEVLCMISRRVDRVYMENNELVQINSYLLKHHHHHH

SEQ ID NO: 13 - protein, 629 aa, Artificial Sequence
  Description of Artificial Sequence Synthetic polypeptide
AATMACPGFLWALVISTCLEFSMAAETTQVKKETITKKEATELVSKVRDLMSQKYTGGSQVGQPIYEIKVGETLSKLKIITNIDELEKLVNALGENKELIVTITDKGHITNSANEVVAEATEKYENSADLSAEANSITEKAKTETNGIYKVADVKASYDSAKDKLVITLRDKTDTVTSKTIEIGIGDEKIDLTANPVDSTGTNLDPSTEGFRVNKIVKLGVAGAKNIDDVQLAEITIKNSDLNTVSPQDLYDGYRLTVKGNMVANGTSKSISDISSKDSETGKYKFTIKYTDASGKAIELTVESTNEKDLKDAKAALEGNSKVKLIAGDDRYATAVAIAKQTKYTDNIVIVNSNKLVDGLAATPLAQSKKAPILLASDNEIPKVTLDYIKDIIKKSPSAKIYIVGGESAVSNTAKKQLESVTKNVERLAGDDRHMTSVAVAKAMGSFKDAFVVGAKGEADAMSIAAKAAELKAPIIVNGWNDLSADAIKLMDGKEIGIVGGSNNVSSQIENQLADVDKDRKVQRVEGETRHDTNAKVIETYYGKLDKLYIAKDGYGNNGMLVDALAAGPLAAGKGPILLAKADITDSQRNALSKKLNLGAEVTQIGNGVELTVIQKIAKILGWHHHHHH

SEQ ID NO: 14 - protein, 613 aa, Artificial Sequence
  Description of Artificial Sequence Synthetic polypeptide
IRRAATMACPGFLWALVISTCLEFSMATSSNKSVDLYSDVYIEKYFNRDKVMEVNIEIDESDLKDMNENAIKEEFKVAKVTVDGDTYGNVGIRTKGNSSLISVANSDSDRYSYKINFDKYNTSQSMEGLTQLNLNNCYSDPSYMREFLTYSICEEMGLATPEFAYAKVSINGEYHGLYLAVEGLKESYLENNFGNVTGDLYKSDEGSSLQYKGDDPESYSNLIVESDKKTADWSKITKLLKSLDTGEDIEKYLDVDSVLKNIAINTALLNLDSYQGSFAHNYYLYEQDGVFSMLPWDFNMSFGGFSGFGGGSQSIAIDEPTTGNLEDRPLISSLLKNETYKTKYHKYLEEIVTKYLDSDYLENMTTKLHDMIASYVKEDPTAFYTYEEFEKNITSSIEDSSDNKGFGNKGFDNNNSNNSDSNNNSNSENKRSGNQSDEKEVNAELTSSVVKANTDNETKNKTTNDSESKNNTDKDKSGNDNNQKLEGPMGKGGKSIPGVLEVAEDMSKTIKSQLSGETSSTKQNSGDESSSGIKGSEKFDEDMSGMPEPPEGMDGKMPPGMGNMDKGDMNGKNGNMNMDRNQDNPREAGGFGNRGGGSVSKTTTYFKHHHHHH

SEQ ID NO: 15 - protein, 514 aa, Artificial Sequence
  Description of Artificial Sequence Synthetic polypeptide
IRRAATMACPGFLWALVISTCLEFSMAIRDKEKVDKAKQEQQIVKWKQEIYRNVIQESKDLYDKYLSVNSPNSIVSEKAYSSTRITSSDESIIVAKGSAGAEKINYQFAVSQMAEPAKFTIKLNSSEPIVRQFPPNASGASSLTIGDVNIPISEQDTTSTIVSKINSLCADNDIKASYSEMTGELIISRKQTGSSSDINLKVIGNDNLAQQIANDNGITFANDASGNKVASVYGKNLEADVTDEHGRVTHISKEQNSFNIDNIDYNVNSKGTAKLTSVTDTEEAVKNMQAFVDDYNKLMDKVYGLVTTKKPKDYPPLTDAQKEDMTTEEIEKWEKKAKEGILRNDDELRGFVEDIQSAFFGDGKNIIALRKLGINESENYNKKGQISFNADTFSKALIDDSDKVYKTLAGYSSNYDDKGMFEKLKDIVYEYSGSSTSKLPKKAGIEKTASASENVYSKQIAEQERNISRLVEKMNDKEKRLYAKYSALESLLNQYSSQMNYFSQAQGNHHHHHH

SEQ ID NO: 16 - DNA, 2110 bp, Clostridium difficile
atgagaaaaattatactttatttaaatgatgatacttttatatctaaaaaatatccagataaaaactttagtaatttagattattgcttaataggaagtaaatgttcaaatagttttgtaaaagaaaagttgattacttttttttaagtgagaataccagatatattaaaagacaaaagtatattaaaagcagagttatttattcatattgattcaaataagaatcatatttttaaagaaaaagtagatattgaaattaaaagaataagtgaatattataatttacgaactataacatggaatgatagagtgtctatggaaaatatcaggggatatttaccaattgggataagtgatacatccaactatatttgtttaaatattacgggaactataaaagcatgggcaatgaataaatatcctaattatgggttagctttatctttaaattacccttatcagatttttgaatttacatctagtagggattgtaacaaaccgtatatacttgtaacatttgaagatagaattatagataattgttatcctaaatgtgagtgtcttccaattagaattacaggtccaatgggaccaagaggagcgacaggaagtataggaccaatgggagcaacaggtccaacaggagcaacaggcaattcctctcagccaattgctaacttcctcgtaaatgcaccatctccacaaacactaaataatggaaatgctataacaggttggtaaacaataataggaaatagttcaagtataacagtagatgcaaatggtacgtttacagtacaagaaaatggtgtgtattatatatcagtttcagtagcattacaaccaggttcatcaagtataaatcaatattcttttgctatcctattcccaattttaggaggaaaagatttggcagggcttactactgagccaggaggcggaggagtactttctggatattttgctggttttttatttggggggactacttttacaataaataatttttcatctacaacagtagggatacgaaatgggcaatcagcaggaactgcggctactttgacgatatttagaatagctgatactgttatgacttaaaacgtgtctaaaataatcttaaaaactatttaggttttatttaaatgacaaaagtatttttatatattgagttttacctattttagaatgaataaaataacaataataataaaatatattcataaaaattttaaatttatggatttttatttaactttattatcaatatatgtataataaaaaactgtctcaaatatagatttgagacagttttcgttatttaaaaattttatattatttaaaatttttgattgcagtagttaaattagggactaattgtttttttcttgatacaacacctggtgcaaatgtaccttgaacaccttcaacatcaaatgcgttagcaattaagctatcttctgctttatagattaaataagaaccttctttgattatatctgtaaccgcaagaattagtttgtcataatcagtcgaatttatataagataaaaactcatcttttttagcaaatatagagtctatgtctaaggtaaatacttgtccaataccaactctatgtccactcatattaaattctttaaaatccatatttactatttcttctatagtatattcatctaaagaagtaccgcatttaaacatatccatagcgtatttttccatgtctacttttgctattttacttaattcttcacaagctttcttatccatatcagttgttgttggagacttaaataataatgtatctgataatatagcagataaaagaagcccagctatttcataaggtatctcaacattgttttctttgtacatttgataaattatagtactattgcatccaacaggcataactctaaatgacataggaacatcagtagaaataccaccaagtttatgatggtcaattatttcaactatgtttgcttgttcaattccatcagcactttgagcatattcgttatggtcaactaaaacaacattctttttagatgggtttaatagatgaccttttgaaactaaacctaaaaacttattatcatcatct

SEQ ID NO: 17 - DNA, 1641 bp, Clostridium difficile
atgagtgatatttcaggtccaagtttatatcaagatgtaggtccaacagggccaacaggtgctactggtccaacaggaccgacggggcctagaggcgcaaccggagcgaccggagcaaatggaataacaggaccaacaggaaatacgggagcaaccggggcgaatggaataacaggaccaacaggaaatatgggagcgactggagcaaatggaacaacaggttctacaggaccaacaggaaatacaggagcgactggagcaaatggaataacaggtccaacaggagcaacaggagcaacgggagcaaatggaataacaggtccaaccggaaacaagggagcaacgggagcgaatgggataacaggtccaacaggagcaacaggagcaacgggagcaaatggaataacaggtccaacaggaaatacaggagcaacgggagcaaatggtgcaaccggactaaccggagcaactggggcaacgggagcgaatgggataacaggtccaacaggagcaacaggagcaacgggagcaaatggagtaacaggtgctacaggcccaacaggaaatacaggagcaacaggtccaaccggaagtataggagcaacgggagcaaatggagtaacaggtgccacaggtccaataggagcaacaggtccaaccggagcagtaggagcaacaggtccagatggtttggtaggtccaacaggcccaacaggcccaaccggagcaaccggagcaaatggtttggtaggtccaacaggcccaaccggagcaaccggagcaaatggtttggtaggtccaacaggagcgaccggagcaacaggagtagctggggcaataggtccaaccggagcagtaggagcgacaggcccaacgggagcagatggagcagtaggtccaaccggagcaaccggagcaacaggggcaaatggagcaacaggcccaacgggagcagtaggagcaactggagcgaatggagtagcaggtccaataggtccaacaggtccaaccggagcaaatggagtagcaggagcaacaggagcgaccggagcaacaggggcaaatggagcaacaggcccaacaggagcagtaggagcaacgggagcaaatggagtagcaggtccaataggtccaacaggaccaacaggagcaaatggaacgaccggagcaacaggggcgaccggagcaacgggagcaaatggagcaacaggtccaacaggagcgaccggagcaacaggagtgttagcagcaaacaatgcacaatttacagtatcttcttcaagtttagggaataatacattagtgacatttaattcatcatttataaatggaactaatataacttttccaacaagtagtactataaatcttgcagttggagggatatacaatgtatctttcggtatacgtgccatactttcacttgcaggatttatgtcaattactactaactttaatggagtagcccaaaataactttattgcaaaagcagtaaatacgcttacttcatcagatgtaagtgtaagtttaagctttttagttgatgctagagcagcagctgttactttaagctttacatttggttcaggcacgacaggtacttctccagctgggtatgtatcagtttatagaatacaatag

SEQ ID NO: 18 - DNA, 2037 bp, Clostridium difficile
atgagtagaaataaatattttggaccatttgatgataatgattacaacaatggctatgataaatatgatgattgtaataatggtcgtgatgattataatagctgtgattgccatcattgctgtccaccatcatgtgtaggtccaacaggcccaatgggtccaagaggtagaaccggcccaacaggaccaacgggtccaacaggtccaggagtagggggaacaggcccaacaggaccaaccggtccgactggcccaacaggaaatacagggaatacaggagcaacaggattaagaggtccaacaggagcaacagggggaacaggcccaacaggagcgacaggagctatagggtttggagtaacaggcccaacaggcccaacaggcccaacaggagcgacaggagcaacaggagcagatggagtaacaggtccaacaggtccaacgggagcaacaggagcagatggaataacaggtccaacaggagcaacaggggcaacaggatttggagtaacaggtccaacaggcccaacaggagcaacaggagtaggagtaacaggagcaacaggattaataggtccaacaggagcgacaggaacacctggagcaacaggtccaacaggggcaataggagcaacaggaataggaataacaggtccaacaggagcaacaggagcaacaggggcagatggagcaacaggagtaacaggcccaacaggcccaacaggggcaacaggagcagatggagtaacaggcccaacaggagcaacaggagcaacaggaataggaataacaggcccaacaggggcaacaggagcaacaggaataggaataacaggagcaacagggttaataggtccaaccggagcaaccggagcaaccggagcaacaggcccaacaggagtaacaggggcaacaggagcagcaggactaataggaccaaccggggcaacaggagtaaccggagcagatggagcaacaggagcgacaggggcaaccggagcaacaggtccaacaggagcagatggattagtaggtccaacaggagcaacaggggcaacaggagcagatggattagtaggcccaacaggtccaacaggggcaaccggagtaggaataactggagcaaccggagcaacaggagcgacaggtccaacaggagcagatggattagtaggtccaaccggagcgacgggagcaacaggagcagatggagtagcaggtccaaccggagcaacaggggcaacaggaaatacaggagcagatggagcaacaggtccaacaggggcaacaggtccaacaggagcagacggattagtaggtccaacaggagcaaccggagcaacaggattagcaggagcaaccggagcaacaggcccaataggagcaacaggtccaacaggagcagatggagcaacaggggcaaccggagcaacaggtccaacaggggcagatggattagtaggtccaaccggagcaacgggagcaacaggggcaacaggtccaacaggcccaacaggtgctagtgcaataataccttttgcatcaggtataccactatcacttacaactatagctggaggattagtaggtacaccaggttttgttggctttggtagttcagctccaggattaagtatagttggtggagtaatagaccttacaaacgcagcagggacattgactaactttgcattttcaatgccaagagatggaacaataacatctatttcagcatacttcagtacaacagcagcactttcacttgttggttcaacaattacaattacagcaacactttaccaatctactgcaccaaataactcatttacagctgtaccaggagcgacagttacactagctccaccacttacaggtatattatcagttggttcaatttctagtggaattgtaacaggattaaatatagcagcaacagcagaaactcgattcttactagtatttactgcaacagcttcaggtctttcattagttaatactgtagcaggatatgcaagtgcaggaattgcaataaattag

SEQ ID NO: 19 - DNA, 1158 bp, Clostridium difficile
atgcaaaaaataacagtgcctacatgggcagagataaatctagataacttaagatttaacttaaataatattaaaaatttattagaagaagatattaagatttgtggagtaataaaagctgatgcatatggacatggtgcagtagaagttgcaaaattgctagaaaaagaaaaagtagattacttagcagtagcaagaactgctgaaggaattgaacttagacaaaatggcataacacttcctattttgaacttgggatatactccagacgaagcttttgaagattctataaaaaataaaataactatgacagtttattctttagaaacagcacaaaagataaatgaaattgcaaaatctttaggagaaaaagcctgtgttcatgttaaaatagactcagggatgactagaataggtttccaacctaatgaggagtcagtacaggaaataatagaattaaataaattagaatatatcgatttagaaggtatgtttactcattttgctacagctgatgaagtaagtaaagagtacacttataaacaagctaataattataaatttatgtctgataaattagatgaggctggtgtaaaaatagctataaaacatgtatcaaacagtgcagctattatggattgccctgatttaagattaaatatggtaagagcaggaataatattatatggtcattatccatctgatgatgtatttaaagatagattagaattaagaccagccatgaaattaaaatcaaaaatcggacatataaaacaagttgaaccaggtgtaggaataagttatggactaaaatacacaactacaggtaaagaaacaatagctacagttccaataggatacgcagatggatttactagaatccaaaaaaatccaaaggttcttattaagggagaagtgtttgatgtagttggtagaatatgtatggatcaaataatggttagaattgacaaagatatagacataaaagttggagatgaggttatactatttggagaaggcgaagttacagctgagcgtatagctaaagacttaggaactataaactatgaagtgttatgtatgatatcaagaagagttgaccgtgtttatatggaaaataatgagcttgtacaaataaacagttatttgctaaaataa

SEQ ID NO: 20 - DNA, 1872 bp, Clostridium difficile
atgaataaaaaaaatctttctgtaattatggctgctgcaatgataagtacatcagtagctccagtttttgctgcagaaactacacaggtaaaaaaagaaacaataactaagaaagaagctacagaactagtttcgaaagttagagatttaatgtctcaaaagtatactggtggttctcaagttggacaaccaatatatgaaataaaagttggcgagactttatcaaaattaaaaataataactaatatagatgaattagagaaattagtaaatgctttgggagaaaataaagaacttattgtaactataacagataaagggcatataacaaatagtgcaaatgaagtagttgcagaagcaactgaaaaatatgaaaattcagcagacctttccgctgaggctaattctataacagaaaaagctaaaactgaaactaatggaatttataaagttgcagatgtaaaagcttcatatgatagtgctaaagataagttagttataactttaagagataaaacagacacagtaacttctaaaactatagagataggtattggtgatgaaaaaattgatttaacagcaaatccagttgattcaacgggaacaaacttagacccttctacagaaggatttagagtaaataaaatcgttaaactaggtgtagcaggagctaaaaatattgatgatgtccaattagctgaaataactataaaaaatagtgacctaaatacagtttcaccacaagatttatatgatggatatagattaactgttaaaggtaatatggtagcaaatggaacatcaaagtcaattagtgatatttcatcaaaagattcagaaacaggaaagtataaatttactattaagtatactgatgcatctggaaaagcaatagagcttactgtagaaagtactaatgaaaaagatttaaaagatgccaaagctgcattagaaggtaattcaaaggttaaattgatagctggagatgatagatatgcaactgcagtggctatagcaaaacaaacaaaatatactgacaatatagttatagttaattcaaataaactagttgatggattagcagctacaccacttgctcaatctaaaaaagcacctatattattagcatccgataatgaaataccaaaagtaactttagattatataaaagatataattaagaaaagcccatcagctaaaatatatatagtaggtggagaatcagcagtatcaaatacagctaaaaagcaattagaatcagtaactaagaatgttgaaagactagctggagatgatagacatatgacttctgtagcagtagcaaaagctatggggtcttttaaagatgcatttgtagtaggtgcgaaaggggaggctgatgctatgagtatagctgccaaagctgctgaacttaaggctcctataatagtaaatggctggaatgatctttcagcagacgctatcaaattgatggatggaaaagagattggtatagttggtggttctaacaatgtatctagtcaaattgaaaatcaacttgctgatgttgataaagatagaaaagttcaaagagttgaaggagaaacaagacacgatactaatgctaaggttatagaaacatattatggaaaattagataaactatatatagcaaaagatggatatggaaataatggtatgctagtagatgcattggcagcaggacctctagcagcaggtaaaggtccaatacttctagctaaagctgatataacagactcacaaaggaatgcacttagtaaaaaattaaatcttggtgcagaagtaactcaaataggtaatggagttgaattgacagtaatacaaaagatagctaaaatactaggttggtaa

SEQ ID NO: 21 - DNA, 2277 bp, Clostridium difficile
atgaataagaagaatatagcaatagctatgtcaggattaacagtattagcttctgcagcacctgtgtttgcagcagaagatatgtcgaaagttgagactggtgatcaaggatatacagtagtacagagcaagtataagaaagcagttgaacaattacaaaaagggttattagatggaagtataacagagattaaaattttctttgagggaactttagcatctactataaaagtaggagctgagcttagtgcagaagatgcaagtaaattattgtttacacaagtagataataaattagacaatttaggtgatggggattatgtagatttcttaataagctctccagcagagggagataaagtaactacaagtaaacttgttgcattaaaaaatttaacaggtggaactagtgcaataaaagtagctacaagtagtattattggtgaagtcgaaaatgctggtactccgggagcaaaaaatacagctccaagtagtgctgcagttatgtctatgtcagatgtatttgatacagcttttacagattcaactgaaactgctgtgaaacttactataaaagatgctatgaaaactaaaaagtttggtttagttgatggaactacttattcaacaggtcttcaatttgcagatggaaaaacagaaaaaattgttaaattaggagatagtgatactataaatttagccaaagaattaataataacacctgcaagtgcaaatgatcaagctgcgactattgagtttgctaaaccaacaacacaatctggaagcccagtaataactaaacttagaatattgaatgcaaaagaagagacaatagatattgatgctagttctagtaaaacagcacaagatttagctaaaaaatatgtatttaataaaacagatttaaatactctttacagagtattaaatggggatgaagcagatactaatagattagtagaagaagttagtggaaaatatcaagtggttctttatccagaaggaaaaagagttacaactaagagtgctgcaaaggcttcaattgctgatgaaaattcaccagttaaattaactcttaagtcagataagaagaaagacttaaaagattatgtggatgatttaagaacatataataatggatattcaaatgctatagaagtagcaggagaagatagaatagaaactgcaatagcattaagtcaaaaatattataactctgatgatgaaaatgctatatttagagattcagttgataatgtagtattggttggaggaaatgcaatagttgatggacttgtagcttctcctttagcttctgaaaagaaagctcctttattattaacttcaaaagataaattagattcaagcgtaaaagctgaaataaagagagttatgaatataaagagtacaacaggtataaatacttcaaagaaagtttatttagctggtggagttaattctatatctaaagaagtagaaaatgaattaaaagatatgggacttaaagttacaagattagcaggagatgatagatatgaaacttctctaaaaatagctgatgaagtaggtcttgataatgataaagcatttgtagttggaggaacaggattagcagatgccatgagtatagctccagttgcatctcaattaagaaatgctaatggtaaaatggatttagctgatggtgatgctacaccaatagtagttgtagatggaaaagctaaaactataaatgatgatgtaaaagatttcttagatgattcacaagttgatataataggtggagaaaacagtgtatctaaagatgttgaaaatgcaatagatgatgctacaggtaaatctccagatagatatagtggagatgatagacaagcaactaatgcaaaagttataaaagaatcttcttattatcaagataacttaaataatgataaaaaagtagttaatttctttgtagctaaagatggttctactaaagaagatcaattagttgatgctttagcagcagctccagttgcagcaaactttggtgtaactcttaattctgatggtaagccagtagataaagatggtaaagtattaactggttctgataatgataaaaataaattagtatctccagcacctatagtattagctactgattctttatcttcagatcaaagtgtatctataagtaaagttcttgataaagataatggagaaaacttagttcaagttggtaaaggtatagctacttcagttataaataaattaaaagatttattaagtatgtaa

SEQ ID NO: 22 - DNA, 1908 bp, Clostridium difficile
atgaaagataaaaaatttacccttcttatctcgattatgattgtatttttatgtgctgtagttggagtttatagtacatctagcaacaaaagtgttgatttatatagtgatgtatatattgaaaaatattttaacagagacaaggttatggaagttaatatagagatagatgaaagtgacttgaaggatatgaatgaaaatgctataaaagaagaatttaaggttgcaaaagtaactgtagatggagatacatatggaaacgtaggtataagaactaaaggaaattcaagtcttatatctgtagcaaatagtgatagtgatagatacagctataagattaattttgataagtataatactagtcaaagtatggaagggcttactcaattaaatcttaataactgttactctgacccatcttatatgagagagtttttaacatatagtatttgcgaggaaatgggattagcgactccagaatttgcatatgctaaagtctctataaatggcgaatatcatggtttgtatttggcagtagaaggattaaaagagtcttatcttgaaaataattttggtaatgtaactggagacttatataagtcagatgaaggaagctcgttgcaatataaaggagatgacccagaaagttactcaaacttaatcgttgaaagtgataaaaagacagctgattggtctaaaattacaaaactattaaaatctttggatacaggtgaagatattgaaaaatatcttgatgtagattctgtccttaaaaatatagcaataaatacagctttattaaaccttgatagctatcaagggagttttgcccataactattatttatatgagcaagatggagtattttctatgttaccatgggattttaatatgtcatttggtggatttagtggttttggtggaggtagtcaatctatagcaattgatgaacctacgacaggtaatttagaagatagacctctcatatcctcgttattaaaaaatgagacatacaaaacaaaataccataaatatctggaagagatagtaacaaaatacctagattcagactatttagagaatatgacaacaaaattgcatgacatgatagcatcatatgtaaaagaagacccaacagcattttatacttatgaagaatttgaaaaaaatataacatcttcaattgaagattctagtgataataagggatttggtaataaagggtttgacaacaataactctaataacagtgattctaataataattctaatagtgaaaataagcgctctggaaatcaaagtgatgaaaaagaagttaatgctgaattaacatcaagcgtagtcaaagctaatacagataatgaaactaaaaataaaactacaaatgatagtgaaagtaagaataatacagataaagataaaagtggaaatgataataatcaaaagctagaaggtcctatgggtaaaggaggtaagtcaataccaggggttttggaagttgcagaagatatgagtaaaactataaaatctcaattaagtggagaaacttcttcgacaaagcaaaactctggtgatgaaagttcaagtggaattaaaggtagtgaaaagtttgatgaggatatgagtggtatgccagaaccacctgagggaatggatggtaaaatgccaccaggaatgggtaatatggataagggagatatgaatggtaaaaatggcaatatgaatatggatagaaatcaagataatccaagagaagctggaggttttggcaatagaggaggaggctctgtgagtaaaacaacaacatacttcaaattaattttaggtggagcttcaatgataataatgtcgattatgttagttggtgtatcaagggtaaagagaagaagatttataaagtcaaaataa

SEQ ID NO: 23 - DNA, 969 bp, Clostridium difficile
atggaaaagagaaaagtaataattgattgtgacccaggaattgatgattctttggcaattcttctggctttaaactcaccagagctagaagtaattggaattaccacatgttgtggaaatgttccagcaaatataggtgcagaaaatgcactaaaaacacttcaaatgtgttcttcactaaatattccagtatatataggagaagaagcaccactaaaaagaaaacttgtaacagctcaagatacacatggagaagatggtattggagaaaacttttatcaaaaggttgtaggagctaaagcaaaaaatggagcagtggattttataataaatactttacataatcatgaaaaagtatcaataatagcacttgcaccacttacaaatatagctaaagcacttattaaagataagaaagcatttgaaaatctcgatgagtttgtatctatgggaggagcatttaggattcatggaaattgctctccagtagcagagtttaattattgggtagacccacatggagcagattatgtttacaagaatttatctaaaaaaatccacatggtaggtttagatgtaactagaaaaattgtacttactcctaatattattgagtttataaatagacttgataagaagatggcaaagtatataactgaaataactagattttatattgatttccattgggaacaggaaggaataattggctgtgtgataaatgaccctctagcagtagcgtactttatagacagaagtatatgtaaaggatttgaatcatatgtagaagttgtagaagatggaatagctatgggtcagtctatagtggattctttcaatttctataaaaaaaatcctaatgcaattgttctaaatgaagttgatgagaagaaatttatgtacatgtttttaaagaggctttttaaaggttatgaagacattatagactctgtggaaggagtgatatag

SEQ ID NO: 24 - DNA, 705 bp, Clostridium difficile
atgaagaaaaaaatattaataccagttattatgtctttatttataatctcacagtgcataacttcatttgcttttacacctgaaaataacaaatttaaggttaaaccattaccttatgcatatgatgcacttgaaccttatatagataaagaaacaatgaaactgcatcatgataagcattatcaagcttatgttgataaattaaatgctgctcttgaaaaatatcctgagctttataattattctttatgtgaattattgcaaaatttagattctttacctaaagatattgctacaactgtaagaaataatgcaggtggagcttataatcataaattcttttttgatataatgacgccagaaaaaaccataccttctgaatctttaaaagaagctattgatagagactttggttcttttgaaaaatttaagcaagagttccaaaaatctgctttagatgtctttggttctggttgggcttggcttgtagctactaaagatgggaaattatctattatgactactccaaatcaggatagccctgtaagtaaaaacctaactcctataataggacttgatgtttgggagcatgcttactatttaaaatatcaaaatagaagaaatgaatacattgacaactggtttaatgtagtaaattggaatggtgctttagaaaattacaaaaatttaaaatctcaagattaa

SEQ ID NO: 25 - DNA, 1000 bp, Clostridium difficile
atgtcaagtataagtccagtaagagttacaggtctttcaggaaattttgatatggaaggcataatcgaagctagtatgattagagacaaggaaaaagttgataaagcaaaacaagaacaacaaatcgttaaatggaagcaagaaatatatagaaatgttatacaagaatcaaaagatctttatgataaatatctaagcgtaaattctcctaatagtatagtaagtgaaaaagcatactcttctacaagaataaccagttctgatgaaagtattatagtagcaaaaggctcagctggtgcagaaaaaataaattatcaatttgcagtttctcaaatggctgaaccagcaaaatttactattaaattaaattcaagtgaacctattgttcgacagttccctccaaatgccagtggagctagttctttaactataggagatgtaaatataccaatatctgaacaagatactacaagtactattgtaagtaagataaactccctttgcgcagataatgatataaaggcttcttatagtgagatgacaggtgaattgattatttcgagaaaacaaactggttcgtcatcagacattaatttaaaagtaattggaaatgacaatttagctcagcaaattgctaatgataatggtatcacatttgcaaatgatgctagtggaaacaaagtggcaagtgtatatggaaaaaatctagaagctgatgtaactgatgaacatggaagagtaactcatataagtaaagaacaaaattcatttaatatagataatattgactataatgtaaattcaaaaggaactgcaaagttgacttctgtcactgatactgaagaagctgttaaaaatatgcaagcatttgtggatgattataataaactgatggacaaggtctatggtttagttactactaaaaaaccaaaagattatccgcctcttacagatgcccaaaaagaagatatgacaactgaagaaatagaaaaatgggaaa

SEQ ID NO: 26 - DNA, 1631 bp, Artificial Sequence
  Description of Artificial Sequence Synthetic polynucleotide
ggatccggcgcgccgccaccatggcatgccctggcttcctgtgggcacttgtgatctccacctgtcttgaattttccatggctatgagtagaaataaatattttggaccatttgatgataatgattacaacaatggctatgataaatatgatgattgtaataatggtcgtgatgattataatagctgtgattgccatcattgctgtccaccatcatgtgtaggtccaacaggcccaatgggtccaagaggtagaaccggcccaacaggaccaacgggtccaacaggtccaggagtagggggaacaggcccaacaggaccaaccggtccgactggcccaacaggaaatacagggaatacaggagcaacaggattaagaggtccaacaggagcaacagggggaacaggcccaacaggagcgacaggagctatagggtttggagtaacaggcccaacaggcccaacaggcccaacaggagcgacaggagcaacaggagcagatggagtaacaggtccaacaggtccaacgggagcaacaggagcagatggaataacaggtccaacaggagcaacaggggcaacaggatttggagtaacaggtccaacaggcccaacaggagcaacaggagtaggagtaacaggagcaacaggattaataggtccaacaggagcgacaggaacacctggagcaacaggtccaacaggggcaataggagcaacaggaataggaataacaggtccaacaggagcaacaggagcaacaggggcagatggagcaacaggagtaacaggcccaacaggcccaacaggggcaacaggagcagatggagtaacaggcccaacaggagcaacaggagcaacaggaataggaataacaggcccaacaggggcaacaggagcaacaggaataggaataacaggagcaacagggttaataggtccaaccggagcaaccggagcaaccggagcaacaggcccaacaggagtaacaggggcaacaggagcagcaggactaataggaccaaccggggcaacaggagtaaccggagcagatggagcaacaggagcgacaggggcaaccggagcaacaggtccaacaggagcagatggattagtaggtccaacaggagcaacaggggcaacaggagcagatggattagtaggcccaacaggtccaacaggggcaaccggagtaggaataactggagcaaccggagcaacaggagcgacaggtccaacaggagcagatggattagtaggtccaaccggagcgacgggagcaacaggagcagatggagtagcaggtccaaccggagcaacaggggcaacaggaaatacaggagcagatggagcaacaggtccaacaggggcaacaggtccaacaggagcagacggattagtaggtccaacaggagcaaccggagcaacaggattagcaggagcaaccggagcaacaggcccaataggagcaacaggtccaacaggagcagatggagcaacaggggcaaccggagcaacaggtccaacaggggcagatggattagtaggtccaaccggagcaacgggagcaacaggggcaacaggtccaacaggcccacatcaccatcaccatcactgataggttaacgctagc

SEQ ID NO: 27 - DNA, 1274 bp, Artificial Sequence
  Description of Artificial Sequence Synthetic polynucleotide
ggatccggcgcgccgccaccatggcatgccctggcttcctgtgggcacttgtgatctccacctgtcttgaattttccatggctatgcaaaaaataacagtgcctacatgggcagagataaatctagataacttaagatttaacttaaataatattaaaaatttattagaagaagatattaagatttgtggagtaataaaagctgatgcatatggacatggtgcagtagaagttgcaaaattgctagaaaaagaaaaagtagattacttagcagtagcaagaactgctgaaggaattgaacttagacaaaatggcataacacttcctattttgaacttgggatatactccagacgaagcttttgaagattctataaaaaataaaataactatgacagtttattctttagaaacagcacaaaagataaatgaaattgcaaaatctttaggagaaaaagcctgtgttcatgttaaaatagactcagggatgactagaataggtttccaacctaatgaggagtcagtacaggaaataatagaattaaataaattagaatatatcgatttagaaggtatgtttactcattttgctacagctgatgaagtaagtaaagagtacacttataaacaagctaataattataaatttatgtctgataaattagatgaggctggtgtaaaaatagctataaaacatgtatcaaacagtgcagctattatggattgccctgatttaagattaaatatggtaagagcaggaataatattatatggtcattatccatctgatgatgtatttaaagatagattagaattaagaccagccatgaaattaaaatcaaaaatcggacatataaaacaagttgaaccaggtgtaggaataagttatggactaaaatacacaactacaggtaaagaaacaatagctacagttccaataggatacgcagatggatttactagaatccaaaaaaatccaaaggttcttattaagggagaagtgtttgatgtagttggtagaatatgtatggatcaaataatggttagaattgacaaagatatagacataaaagttggagatgaggttatactatttggagaaggcgaagttacagctgagcgtatagctaaagacttaggaactataaactatgaagtgttatgtatgatatcaagaagagttgaccgtgtttatatggaaaataatgagcttgtacaaataaacagttatttgctaaaacatcaccatcaccatcactgataggttaacgctagc

SEQ ID NO: 28 - DNA, 1916 bp, Artificial Sequence
  Description of Artificial Sequence Synthetic polynucleotide
ggatccggcgcgccgccaccatggcatgccctggcttcctgtgggcacttgtgatctccacctgtcttgaattttccatggctgcagaaactacacaggtaaaaaaagaaacaataactaagaaagaagctacagaactagtttcgaaagttagagatttaatgtctcaaaagtatactggtggttctcaagttggacaaccaatatatgaaataaaagttggcgagactttatcaaaattaaaaataataactaatatagatgaattagagaaattagtaaatgctttgggagaaaataaagaacttattgtaactataacagataaagggcatataacaaatagtgcaaatgaagtagttgcagaagcaactgaaaaatatgaaaattcagcagacctttccgctgaggctaattctataacagaaaaagctaaaactgaaactaatggaatttataaagttgcagatgtaaaagcttcatatgatagtgctaaagataagttagttataactttaagagataaaacagacacagtaacttctaaaactatagagataggtattggtgatgaaaaaattgatttaacagcaaatccagttgattcaacgggaacaaacttagacccttctacagaaggatttagagtaaataaaatcgttaaactaggtgtagcaggagctaaaaatattgatgatgtccaattagctgaaataactataaaaaatagtgacctaaatacagtttcaccacaagatttatatgatggatatagattaactgttaaaggtaatatggtagcaaatggaacatcaaagtcaattagtgatatttcatcaaaagattcagaaacaggaaagtataaatttactattaagtatactgatgcatctggaaaagcaatagagcttactgtagaaagtactaatgaaaaagatttaaaagatgccaaagctgcattagaaggtaattcaaaggttaaattgatagctggagatgatagatatgcaactgcagtggctatagcaaaacaaacaaaatatactgacaatatagttatagttaattcaaataaactagttgatggattagcagctacaccacttgctcaatctaaaaaagcacctatattattagcatccgataatgaaataccaaaagtaactttagattatataaaagatataattaagaaaagcccatcagctaaaatatatatagtaggtggagaatcagcagtatcaaatacagctaaaaagcaattagaatcagtaactaagaatgttgaaagactagctggagatgatagacatatgacttctgtagcagtagcaaaagctatggggtcttttaaagatgcatttgtagtaggtgcgaaaggggaggctgatgctatgagtatagctgccaaagctgctgaacttaaggctcctataatagtaaatggctggaatgatctttcagcagacgctatcaaattgatggatggaaaagagattggtatagttggtggttctaacaatgtatctagtcaaattgaaaatcaacttgctgatgttgataaagatagaaaagttcaaagagttgaaggagaaacaagacacgatactaatgctaaggttatagaaacatattatggaaaattagataaactatatatagcaaaagatggatatggaaataatggtatgctagtagatgcattggcagcaggacctctagcagcaggtaaaggtccaatacttctagctaaagctgatataacagactcacaaaggaatgcacttagtaaaaaattaaatcttggtgcagaagtaactcaaataggtaatggagttgaattgacagtaatacaaaagatagctaaaatactaggttggcatcaccatcaccatcactgataggttaacgctagc

SEQ ID NO: 29 - DNA, 1859 bp, Artificial Sequence
  Description of Artificial Sequence Synthetic polynucleotide
ggatccggcgcgccgccaccatggcatgccctggcttcctgtgggcacttgtgatctccacctgtcttgaattttccatggctacatctagcaacaaaagtgttgatttatatagtgatgtatatattgaaaaatattttaacagagacaaggttatggaagttaatatagagatagatgaaagtgacttgaaggatatgaatgaaaatgctataaaagaagaatttaaggttgcaaaagtaactgtagatggagatacatatggaaacgtaggtataagaactaaaggaaattcaagtcttatatctgtagcaaatagtgatagtgatagatacagctataagattaattttgataagtataatactagtcaaagtatggaagggcttactcaattaaatcttaataactgttactctgacccatcttatatgagagagtttttaacatatagtatttgcgaggaaatgggattagcgactccagaatttgcatatgctaaagtctctataaatggcgaatatcatggtttgtatttggcagtagaaggattaaaagagtcttatcttgaaaataattttggtaatgtaactggagacttatataagtcagatgaaggaagctcgttgcaatataaaggagatgacccagaaagttactcaaacttaatcgttgaaagtgataaaaagacagctgattggtctaaaattacaaaactattaaaatctttggatacaggtgaagatattgaaaaatatcttgatgtagattctgtccttaaaaatatagcaataaatacagctttattaaaccttgatagctatcaagggagttttgcccataactattatttatatgagcaagatggagtattttctatgttaccatgggattttaatatgtcatttggtggatttagtggttttggtggaggtagtcaatctatagcaattgatgaacctacgacaggtaatttagaagatagacctctcatatcctcgttattaaaaaatgagacatacaaaacaaaataccataaatatctggaagagatagtaacaaaatacctagattcagactatttagagaatatgacaacaaaattgcatgacatgatagcatcatatgtaaaagaagacccaacagcattttatacttatgaagaatttgaaaaaaatataacatcttcaattgaagattctagtgataataagggatttggtaataaagggtttgacaacaataactctaataacagtgattctaataataattctaatagtgaaaataagcgctctggaaatcaaagtgatgaaaaagaagttaatgctgaattaacatcaagcgtagtcaaagctaatacagataatgaaactaaaaataaaactacaaatgatagtgaaagtaagaataatacagataaagataaaagtggaaatgataataatcaaaagctagaaggtcctatgggtaaaggaggtaagtcaataccaggggttttggaagttgcagaagatatgagtaaaactataaaatctcaattaagtggagaaacttcttcgacaaagcaaaactctggtgatgaaagttcaagtggaattaaaggtagtgaaaagtttgatgaggatatgagtggtatgccagaaccacctgagggaatggatggtaaaatgccaccaggaatgggtaatatggataagggagatatgaatggtaaaaatggcaatatgaatatggatagaaatcaagataatccaagagaagctggaggttttggcaatagaggaggaggctctgtgagtaaaacaacaacatacttcaaacatcaccatcaccatcactgataggttaacgctagc

SEQ ID NO: 30 - DNA, 1562 bp, Artificial Sequence
  Description of Artificial Sequence Synthetic polynucleotide
ggatccggcgcgccgccaccatggcatgccctggcttcctgtgggcacttgtgatctccacctgtcttgaattttccatggctattagagacaaggaaaaagttgataaagcaaaacaagaacaacaaatcgttaaatggaagcaagaaatatatagaaatgttatacaagaatcaaaagatctttatgataaatatctaagcgtaaattctcctaatagtatagtaagtgaaaaagcatactcttctacaagaataaccagttctgatgaaagtattatagtagcaaaaggctcagctggtgcagaaaaaataaattatcaatttgcagtttctcaaatggctgaaccagcaaaatttactattaaattaaattcaagtgaacctattgttcgacagttccctccaaatgccagtggagctagttctttaactataggagatgtaaatataccaatatctgaacaagatactacaagtactattgtaagtaagataaactccctttgcgcagataatgatataaaggcttcttatagtgagatgacaggtgaattgattatttcgagaaaacaaactggttcgtcatcagacattaatttaaaagtaattggaaatgacaatttagctcagcaaattgctaatgataatggtatcacatttgcaaatgatgctagtggaaacaaagtggcaagtgtatatggaaaaaatctagaagctgatgtaactgatgaacatggaagagtaactcatataagtaaagaacaaaattcatttaatatagataatattgactataatgtaaattcaaaaggaactgcaaagttgacttctgtcactgatactgaagaagctgttaaaaatatgcaagcatttgtggatgattataataaactgatggacaaggtctatggtttagttactactaaaaaaccaaaagattatccgcctcttacagatgcccaaaaagaagatatgacaactgaagaaatagaaaaatgggaaaagaaagctaaagaaggtatacttagaaatgatgatgagttaagaggttttgttgaagatattcagtctgcattttttggagatggaaaaaatattattgcattaagaaaactaggtatcaatgaaagcgaaaattacaataaaaaaggtcaaatatcatttaatgcagatactttttcaaaggctcttatagatgatagtgataaggtatacaaaacactagcaggttattcttcgaattatgatgataagggaatgtttgaaaagctaaaagatattgtatatgaatattctggaagttcaacttctaaacttcctaaaaaagcaggtatagaaaaaactgcttctgctagtgaaaatgtatattcaaaacaaattgcagagcaagaaagaaatataagcaggttagttgaaaaaatgaatgataaagagaaaagactttatgctaaatattcagccttagaatctttgttgaatcagtattcttcccaaatgaattatttctcacaagcacagggtaatcatcaccatcaccatcactgataggttaacgctagc